SEQUENCE LISTING (137 sequences)

SEQ ID NO: 1 - DNA, 350 bp, Homo sapiens
  misc_feature 1..350: note = Description of sequence: CAGE region: EGFR
  source 1..350: mol_type = genomic DNA
cctcgcattctcctcctcctctgctcctcccgatccctcctccgccgcctggtccctcctcctcccgccctgcctccccgcgcctcggcccgcgcgagctagacgtccgggcagcccccggcgcagcgcggccgcagcagcctccgccccccgcacggtgtgagcgcccgacgcggccgaggcggccggagtcccgagctagccccggcggccgccgccgcccagaccggacgacaggccacctcgtcggcgtccgcccgagtccccgcctcgccgccaacgccacaaccaccgcgcacggccccctgactccgtccagtattgatcgggagagccggagcgagctcttc

SEQ ID NO: 2 - DNA, 350 bp, Homo sapiens
  misc_feature 1..350: note = Description of sequence: CAGE region: BRAF (Long isoform)
  source 1..350: mol_type = genomic DNA
accgtgacacgcgggtgacgccgttgccgcggcgacttctcgtcgtctccgcccccttcccccgctcccccccgcacccccgcctagcgtccttcccccaatcccctcaggctcggctgcgcccggggccgcgggccggtacctgaggtggcccaggcgccctccgcccgcggcgccgcccgggccgctcctccccgcgccccccgcgccccccgctcctccgcctccgcctccgcctccgcctcccccagctctccgcctcccttccccctccccgcccgacagcggccgctcgggccccggctctcggttataagatggcggcgctgagcggtggcggtggtggcggc

SEQ ID NO: 3 - DNA, 350 bp, Homo sapiens
  misc_feature 1..350: note = Description of sequence: CAGE region: BRAF (Short isoform)
  source 1..350: mol_type = genomic DNA
ccccccgctcctccgcctccgcctccgcctccgcctcccccagctctccgcctcccttccccctccccgcccgacagcggccgctcgggccccggctctcggttataagatggcggcgctgagcggtggcggtggtggcggcgcggagccgggccaggctctgttcaacggggacatggagcccgaggccggcgccggcgccggcgccgcggcctcttcggctgcggaccctgccattccggaggaggtgagtgctggcgccaccctgccgccctcccgactccgggctcggcggctggctggtgtttattttggaaagaggcggcggtgggggcttgatgccctcagcc

SEQ ID NO: 4 - DNA, 350 bp, Homo sapiens
  misc_feature 1..350: note = Description of sequence: CAGE region: NRAS
  source 1..350: mol_type = genomic DNA
ggtgcggcctgccgcatgactcgtggttcggaggcccacgtggccggggcggggactcaggcgcctggggcgccgactgattacgtagcgggcggggccggaagtgccgctccttggtgggggctgttcatggcggttccggggtctccaacatttttcccggctgtggtcctaaatctgtccaaagcagaggcagtggagcttgaggtaagtttatctcatgcatagtgttcggctttgggctgtggaatgttcaggcgtttcactgatgccagaaatggagcagaatctatcagctggagacaaaggccttgggcgggggtccttccatttggtgcctacgtggggag

SEQ ID NO: 5 - DNA, 350 bp, Homo sapiens
  misc_feature 1..350: note = Description of sequence: CAGE region: Apolipoprotein B-100 (APOB-100)
  source 1..350: mol_type = genomic DNA
aacaggtcaggcccgggaggcgccctttggaccttttgcaatcctggcgctcttgcagcctgggcttcctataaatggggtgcgggcgccggccgcgcattcccaccgggacctgcggggctgagtgcccttctcggttgctgccgctgaggagcccgcccagccagccagggccgcgaggccgaggccaggccgcagcccaggagccgccccaccgcagctggcgatggacccgccgaggcccgcgctgctggcgctgctggcgctgcctgcgctgctgctgctgctgctggcgggcgccagggccggtgagtgcgcggccgctctgcgggcgcagagggagcgggagg

SEQ ID NO: 6 - DNA, 350 bp, Homo sapiens
  misc_feature 1..350: note = Description of sequence: CAGE region: Huntingtin (HTT)
  source 1..350: mol_type = genomic DNA
tagggctgtcaatcatgctggccggcgtggccccgcctccgccggcgcggccccgcctccgccggcgcagcgtctgggacgcaaggcgccgtgggggctgccgggacgggtccaagatggacggccgctcaggttctgcttttacctgcggcccagagccccattcattgccccggtgctgagcggcgccgcgagtcggcccgaggcctccggggactgccgtgccgggcgggagaccgccatggcgaccctggaaaagctgatgaaggccttcgagtccctcaagtccttccagcagcagcagcagcagcagcagcagcagcagcagcagcagcagcagcagcagcagc

SEQ ID NO: 7 - DNA, 350 bp, Homo sapiens
  misc_feature 1..350: note = Description of sequence: CAGE region: Superoxide Dismutase 1 (SOD1)
  source 1..350: mol_type = genomic DNA
gggcgggctgagcgcgtgcgaggcgattggtttggggccagagtgggcgaggcgcggaggtctggcctataaagtagtcgcggagacggggtgctggtttgcgtcgtagtctcctgcagcgtctggggtttccgttgcagtcctcggaaccaggacctcggcgtggcctagcgagttatggcgacgaaggccgtgtgcgtgctgaagggcgacggcccagtgcagggcatcatcaatttcgagcagaaggcaagggctgggacggaggcttgtttgcgaggccgctcccacccgctcgtccccccgcgcacctttgctaggagcgggtcgcccgccaggcctcggggccg

SEQ ID NO: 8 - DNA, 350 bp, Homo sapiens
  misc_feature 1..350: note = Description of sequence: CAGE region: MYC (Longer isoform)
  source 1..350: mol_type = genomic DNA
ggttcccaaagcagagggcgtgggggaaaagaaaaaagatcctctctcgctaatctccgcccaccggccctttataatgcgagggtctggacggctgaggacccccgagctgtgctgctcgcggccgccaccgccgggccccggccgtccctggctcccctcctgcctcgagaagggcagggcttctcagaggcttggcgggaaaaagaacggagggagggatcgcgctgagtataaaagccggttttcggggctttatctaactcgctgtagtaattccagcgagaggcagagggagcgagcgggcggccggctagggtggaagagccgggcgagcagagctgcgctgc

SEQ ID NO: 9 - DNA, 350 bp, Homo sapiens
  misc_feature 1..350: note = Description of sequence: CAGE region: MYC (Shorter isoform)
  source 1..350: mol_type = genomic DNA
tcctgcctcgagaagggcagggcttctcagaggcttggcgggaaaaagaacggagggagggatcgcgctgagtataaaagccggttttcggggctttatctaactcgctgtagtaattccagcgagaggcagagggagcgagcgggcggccggctagggtggaagagccgggcgagcagagctgcgctgcgggcgtcctgggaagggagatccggagcgaatagggggcttcgcctctggcccagccctcccgctgatcccccagccagcggtccgcaacccttgccgcatccacgaaactttgcccatagcagcgggcgggcactttgcactggaacttacaacacccg

SEQ ID NO: 10 - DNA, 18 bp, Homo sapiens
  misc_feature 1..18: note = Description of sequence: TCS-0001 EGFR-1
  source 1..18: mol_type = genomic DNA
gcggaggctgctgcggcc

SEQ ID NO: 11 - DNA, 18 bp, Homo sapiens
  misc_feature 1..18: note = Description of sequence: TCS-0002 EGFR-2
  source 1..18: mol_type = genomic DNA
ggaggctgctgcggccgc

SEQ ID NO: 12 - DNA, 18 bp, Homo sapiens
  misc_feature 1..18: note = Description of sequence: TCS-0003 EGFR-3
  source 1..18: mol_type = genomic DNA
cgtctagctcgcgcgggc

SEQ ID NO: 13 - DNA, 18 bp, Homo sapiens
  misc_feature 1..18: note = Description of sequence: TCS-0004 EGFR-4
  source 1..18: mol_type = genomic DNA
ctcacaccgtgcgggggg

SEQ ID NO: 14 - DNA, 18 bp, Homo sapiens
  misc_feature 1..18: note = Description of sequence: TCS-0005 EGFR-5
  source 1..18: mol_type = genomic DNA
accgtgcggggggcggag

SEQ ID NO: 15 - DNA, 18 bp, Homo sapiens
  misc_feature 1..18: note = Description of sequence: TCS-0006 EGFR-6
  source 1..18: mol_type = genomic DNA
gacgtctagctcgcgcgg

SEQ ID NO: 16 - DNA, 18 bp, Homo sapiens
  misc_feature 1..18: note = Description of sequence: TCS-0007 EGFR-7
  source 1..18: mol_type = genomic DNA
gctgcccggacgtctagc

SEQ ID NO: 17 - DNA, 18 bp, Homo sapiens
  misc_feature 1..18: note = Description of sequence: TCS-0008 EGFR-8
  source 1..18: mol_type = genomic DNA
cacaccgtgcggggggcg

SEQ ID NO: 18 - DNA, 18 bp, Homo sapiens
  misc_feature 1..18: note = Description of sequence: TCS-0009 EGFR-9
  source 1..18: mol_type = genomic DNA
aggctgctgcggccgcgc

SEQ ID NO: 19 - DNA, 18 bp, Homo sapiens
  misc_feature 1..18: note = Description of sequence: TCS-0010 EGFR-10
  source 1..18: mol_type = genomic DNA
tgctgcggccgcgctgcg

SEQ ID NO: 20 - DNA, 18 bp, Homo sapiens
  misc_feature 1..18: note = Description of sequence: TCS-0011 EGFR-11
  source 1..18: mol_type = genomic DNA
gggctgcccggacgtcta

SEQ ID NO: 21 - DNA, 18 bp, Homo sapiens
  misc_feature 1..18: note = Description of sequence: TCS-0012 EGFR-12
  source 1..18: mol_type = genomic DNA
gggggctgcccggacgtc

SEQ ID NO: 22 - DNA, 18 bp, Homo sapiens
  misc_feature 1..18: note = Description of sequence: TCS-0013 EGFR-13
  source 1..18: mol_type = genomic DNA
ggcgggagtggggcggcg

SEQ ID NO: 23 - DNA, 18 bp, Homo sapiens
  misc_feature 1..18: note = Description of sequence: TCS-0014 EGFR-14
  source 1..18: mol_type = genomic DNA
gctgcgccgggggctgcc

SEQ ID NO: 24 - DNA, 18 bp, Homo sapiens
  misc_feature 1..18: note = Description of sequence: TCS-0015 EGFR-15
  source 1..18: mol_type = genomic DNA
tgcccggacgtctagctc

SEQ ID NO: 25 - DNA, 18 bp, Homo sapiens
  misc_feature 1..18: note = Description of sequence: TCS-0016 EGFR-16
  source 1..18: mol_type = genomic DNA
cgggaggaggagggacca

SEQ ID NO: 26 - DNA, 18 bp, Homo sapiens
  misc_feature 1..18: note = Description of sequence: TCS-0017 EGFR-17
  source 1..18: mol_type = genomic DNA
cggacgtctagctcgcgc

SEQ ID NO: 27 - DNA, 18 bp, Homo sapiens
  misc_feature 1..18: note = Description of sequence: TCS-0018 EGFR-18
  source 1..18: mol_type = genomic DNA
gccgggggctgcccggac

SEQ ID NO: 28 - DNA, 18 bp, Homo sapiens
  misc_feature 1..18: note = Description of sequence: TCS-0019 EGFR-19
  source 1..18: mol_type = genomic DNA
cccggacgtctagctcgc

SEQ ID NO: 29 - DNA, 18 bp, Homo sapiens
  misc_feature 1..18: note = Description of sequence: TCS-0020 EGFR-20
  source 1..18: mol_type = genomic DNA
ggcagggcgggaggagga

SEQ ID NO: 30 - DNA, 18 bp, Homo sapiens
  misc_feature 1..18: note = Description of sequence: TCS-0021 EGFR-21
  source 1..18: mol_type = genomic DNA
cgcggggaggcagggcgg

SEQ ID NO: 31 - DNA, 18 bp, Homo sapiens
  misc_feature 1..18: note = Description of sequence: TCS-0022 EGFR-22
  source 1..18: mol_type = genomic DNA
cgggccgaggcgcgggga

SEQ ID NO: 32 - DNA, 18 bp, Homo sapiens
  misc_feature 1..18: note = Description of sequence: TCS-0023 EGFR-23
  source 1..18: mol_type = genomic DNA
ggaggcagggcgggagga

SEQ ID NO: 33 - DNA, 18 bp, Homo sapiens
  misc_feature 1..18: note = Description of sequence: TCS-0024 BRAF-1
  source 1..18: mol_type = genomic DNA
attgggggaaggacgcta

SEQ ID NO: 34 - DNA, 18 bp, Homo sapiens
  misc_feature 1..18: note = Description of sequence: TCS-0025 BRAF-2
  source 1..18: mol_type = genomic DNA
aggggattgggggaagga

SEQ ID NO: 35 - DNA, 18 bp, Homo sapiens
  misc_feature 1..18: note = Description of sequence: TCS-0026 BRAF-3
  source 1..18: mol_type = genomic DNA
gggattgggggaaggacg

SEQ ID NO: 36 - DNA, 18 bp, Homo sapiens
  misc_feature 1..18: note = Description of sequence: TCS-0027 BRAF-4
  source 1..18: mol_type = genomic DNA
ctgaggggattgggggaa

SEQ ID NO: 37 - DNA, 18 bp, Homo sapiens
  misc_feature 1..18: note = Description of sequence: TCS-0028 BRAF-5
  source 1..18: mol_type = genomic DNA
tgggggaaggacgctagg

SEQ ID NO: 38 - DNA, 18 bp, Homo sapiens
  misc_feature 1..18: note = Description of sequence: TCS-0029 BRAF-6
  source 1..18: mol_type = genomic DNA
gagcctgaggggattggg

SEQ ID NO: 39 - DNA, 18 bp, Homo sapiens
  misc_feature 1..18: note = Description of sequence: TCS-0030 BRAF-7
  source 1..18: mol_type = genomic DNA
ccgagcctgaggggattg

SEQ ID NO: 40 - DNA, 18 bp, Homo sapiens
  misc_feature 1..18: note = Description of sequence: TCS-0031 BRAF-8
  source 1..18: mol_type = genomic DNA
gggaaggacgctaggcgg

SEQ ID NO: 41 - DNA, 18 bp, Homo sapiens
  misc_feature 1..18: note = Description of sequence: TCS-0032 BRAF-9
  source 1..18: mol_type = genomic DNA
agccgagcctgaggggat

SEQ ID NO: 42 - DNA, 18 bp, Homo sapiens
  misc_feature 1..18: note = Description of sequence: TCS-0033 BRAF-10
  source 1..18: mol_type = genomic DNA
gcctgaggggattggggg

SEQ ID NO: 43 - DNA, 18 bp, Homo sapiens
  misc_feature 1..18: note = Description of sequence: TCS-0034 BRAF-11
  source 1..18: mol_type = genomic DNA
tcaggtaccggcccgcgg

SEQ ID NO: 44 - DNA, 18 bp, Homo sapiens
  misc_feature 1..18: note = Description of sequence: TCS-0035 BRAF-12
  source 1..18: mol_type = genomic DNA
ccgggcgcagccgagcct

SEQ ID NO: 45 - DNA, 18 bp, Homo sapiens
  misc_feature 1..18: note = Description of sequence: TCS-0036 BRAF-13
  source 1..18: mol_type = genomic DNA
gcagccgagcctgagggg

SEQ ID NO: 46 - DNA, 18 bp, Homo sapiens
  misc_feature 1..18: note = Description of sequence: TCS-0037 BRAF-14
  source 1..18: mol_type = genomic DNA
gcgcagccgagcctgagg

SEQ ID NO: 47 - DNA, 18 bp, Homo sapiens
  misc_feature 1..18: note = Description of sequence: TCS-0038 BRAF-15
  source 1..18: mol_type = genomic DNA
gggcgcagccgagcctga

SEQ ID NO: 48 - DNA, 18 bp, Homo sapiens
  misc_feature 1..18: note = Description of sequence: TCS-0039 BRAF-16
  source 1..18: mol_type = genomic DNA
ggcggagggcgcctgggc

SEQ ID NO: 49 - DNA, 18 bp, Homo sapiens
  misc_feature 1..18: note = Description of sequence: TCS-0040 BRAF-17
  source 1..18: mol_type = genomic DNA
cggagggcgcctgggcca

SEQ ID NO: 50 - DNA, 18 bp, Homo sapiens
  misc_feature 1..18: note = Description of sequence: TCS-0041 BRAF-18
  source 1..18: mol_type = genomic DNA
ccccgggcgcagccgagc

SEQ ID NO: 51 - DNA, 18 bp, Homo sapiens
  misc_feature 1..18: note = Description of sequence: TCS-0042 BRAF-19
  source 1..18: mol_type = genomic DNA
cttataaccgagagccgg

SEQ ID NO: 52 - DNA, 18 bp, Homo sapiens
  misc_feature 1..18: note = Description of sequence: TCS-0043 BRAF-20
  source 1..18: mol_type = genomic DNA
gcgccgccatcttataac

SEQ ID NO: 53 - DNA, 18 bp, Homo sapiens
  misc_feature 1..18: note = Description of sequence: TCS-0044 BRAF-21
  source 1..18: mol_type = genomic DNA
catgtccccgttgaacag

SEQ ID NO: 54 - DNA, 18 bp, Homo sapiens
  misc_feature 1..18: note = Description of sequence: TCS-0045 BRAF-22
  source 1..18: mol_type = genomic DNA
atcttataaccgagagcc

SEQ ID NO: 55 - DNA, 18 bp, Homo sapiens
  misc_feature 1..18: note = Description of sequence: TCS-0046 BRAF-23
  source 1..18: mol_type = genomic DNA
ccccgttgaacagagcct

SEQ ID NO: 56 - DNA, 18 bp, Homo sapiens
  misc_feature 1..18: note = Description of sequence: TCS-0047 NRAS-1
  source 1..18: mol_type = genomic DNA
ccgcccgctacgtaatca

SEQ ID NO: 57 - DNA, 18 bp, Homo sapiens
  misc_feature 1..18: note = Description of sequence: TCS-0048 NRAS-2
  source 1..18: mol_type = genomic DNA
gccatgaacagcccccac

SEQ ID NO: 58 - DNA, 18 bp, Homo sapiens
  misc_feature 1..18: note = Description of sequence: TCS-0049 NRAS-3
  source 1..18: mol_type = genomic DNA
ccccgcccgctacgtaat

SEQ ID NO: 59 - DNA, 18 bp, Homo sapiens
  misc_feature 1..18: note = Description of sequence: TCS-0050 NRAS-4
  source 1..18: mol_type = genomic DNA
catgaacagcccccacca

SEQ ID NO: 60 - DNA, 18 bp, Homo sapiens
  misc_feature 1..18: note = Description of sequence: TCS-0051 NRAS-5
  source 1..18: mol_type = genomic DNA
ggccccgcccgctacgta

SEQ ID NO: 61 - DNA, 18 bp, Homo sapiens
  misc_feature 1..18: note = Description of sequence: TCS-0052 NRAS-6
  source 1..18: mol_type = genomic DNA
caaggagcggcacttccg

SEQ ID NO: 62 - DNA, 18 bp, Homo sapiens
  misc_feature 1..18: note = Description of sequence: TCS-0053 NRAS-7
  source 1..18: mol_type = genomic DNA
cccccaccaaggagcggc

SEQ ID NO: 63 - DNA, 18 bp, Homo sapiens
  misc_feature 1..18: note = Description of sequence: TCS-0054 NRAS-8
  source 1..18: mol_type = genomic DNA
accaaggagcggcacttc

SEQ ID NO: 64 - DNA, 18 bp, Homo sapiens
  misc_feature 1..18: note = Description of sequence: TCS-0055 NRAS-9
  source 1..18: mol_type = genomic DNA
gccgggaaaaatgttgga

SEQ ID NO: 65 - DNA, 18 bp, Homo sapiens
  misc_feature 1..18: note = Description of sequence: TCS-0056 NRAS-10
  source 1..18: mol_type = genomic DNA
gatttaggaccacagccg

SEQ ID NO: 66 - DNA, 18 bp, Homo sapiens
  misc_feature 1..18: note = Description of sequence: TCS-0057 NRAS-11
  source 1..18: mol_type = genomic DNA
aaaatgttggagaccccg

SEQ ID NO: 67 - DNA, 18 bp, Homo sapiens
  misc_feature 1..18: note = Description of sequence: TCS-0058 NRAS-12
  source 1..18: mol_type = genomic DNA
ggcacttccggccccgcc

SEQ ID NO: 68 - DNA, 18 bp, Homo sapiens
  misc_feature 1..18: note = Description of sequence: TCS-0059 NRAS-13
  source 1..18: mol_type = genomic DNA
ttccggccccgcccgcta

SEQ ID NO: 69 - DNA, 18 bp, Homo sapiens
  misc_feature 1..18: note = Description of sequence: TCS-0060 NRAS-14
  source 1..18: mol_type = genomic DNA
aggagcggcacttccggc

SEQ ID NO: 70 - DNA, 18 bp, Homo sapiens
  misc_feature 1..18: note = Description of sequence: TCS-0061 NRAS-15
  source 1..18: mol_type = genomic DNA
gcggcacttccggccccg

SEQ ID NO: 71 - DNA, 18 bp, Homo sapiens
  misc_feature 1..18: note = Description of sequence: TCS-0062 NRAS-16
  source 1..18: mol_type = genomic DNA
gagcggcacttccggccc

SEQ ID NO: 72 - DNA, 18 bp, Homo sapiens
  misc_feature 1..18: note = Description of sequence: TCS-0063 NRAS-17
  source 1..18: mol_type = genomic DNA
ccaccaaggagcggcact

SEQ ID NO: 73 - DNA, 18 bp, Homo sapiens
  misc_feature 1..18: note = Description of sequence: TCS-0064 NRAS-18
  source 1..18: mol_type = genomic DNA
gacagatttaggaccaca

SEQ ID NO: 74 - DNA, 18 bp, Homo sapiens
  misc_feature 1..18: note = Description of sequence: TCS-0065 NRAS-19
  source 1..18: mol_type = genomic DNA
cctctgctttggacagat

SEQ ID NO: 75 - DNA, 18 bp, Homo sapiens
  misc_feature 1..18: note = Description of sequence: TCS-0066 NRAS-20
  source 1..18: mol_type = genomic DNA
cggaaccgccatgaacag

SEQ ID NO: 76 - DNA, 18 bp, Homo sapiens
  misc_feature 1..18: note = Description of sequence: TCS-0067 NRAS-21
  source 1..18: mol_type = genomic DNA
gctttggacagatttagg

SEQ ID NO: 77 - DNA, 18 bp, Homo sapiens
  misc_feature 1..18: note = Description of sequence: TCS-0068 NRAS-22
  source 1..18: mol_type = genomic DNA
gaacagcccccaccaagg

SEQ ID NO: 78 - DNA, 18 bp, Homo sapiens
  misc_feature 1..18: note = Description of sequence: TCS-0069 NRAS-23
  source 1..18: mol_type = genomic DNA
cagcccccaccaaggagc

SEQ ID NO: 79 - DNA, 18 bp, Homo sapiens
  misc_feature 1..18: note = Description of sequence: TCS-0070 APOB-1
  source 1..18: mol_type = genomic DNA
gccccgcaggtcccggtg

SEQ ID NO: 80 - DNA, 18 bp, Homo sapiens
  misc_feature 1..18: note = Description of sequence: TCS-0071 APOB-2
  source 1..18: mol_type = genomic DNA
ctcagccccgcaggtccc

SEQ ID NO: 81 - DNA, 18 bp, Homo sapiens
  misc_feature 1..18: note = Description of sequence: TCS-0072 APOB-3
  source 1..18: mol_type = genomic DNA
accgagaagggcactcag

SEQ ID NO: 82 - DNA, 18 bp, Homo sapiens
  misc_feature 1..18: note = Description of sequence: TCS-0073 APOB-4
  source 1..18: mol_type = genomic DNA
cggcagcaaccgagaagg

SEQ ID NO: 83 - DNA, 18 bp, Homo sapiens
  misc_feature 1..18: note = Description of sequence: TCS-0074 APOB-5
  source 1..18: mol_type = genomic DNA
tcagcggcagcaaccgag

SEQ ID NO: 84 - DNA, 18 bp, Homo sapiens
  misc_feature 1..18: note = Description of sequence: TCS-0075 HTT-1
  source 1..18: mol_type = genomic DNA
caatgaatggggctctgg

SEQ ID NO: 85 - DNA, 18 bp, Homo sapiens
  misc_feature 1..18: note = Description of sequence: TCS-0076 HTT-2
  source 1..18: mol_type = genomic DNA
ggcaatgaatggggctct

SEQ ID NO: 86 - DNA, 18 bp, Homo sapiens
  misc_feature 1..18: note = Description of sequence: TCS-0077 HTT-3
  source 1..18: mol_type = genomic DNA
atgaatggggctctgggc

SEQ ID NO: 87 - DNA, 18 bp, Homo sapiens
  misc_feature 1..18: note = Description of sequence: TCS-0078 HTT-4
  source 1..18: mol_type = genomic DNA
cgcaggtaaaagcagaac

SEQ ID NO: 88 - DNA, 18 bp, Homo sapiens
  misc_feature 1..18: note = Description of sequence: TCS-0079 HTT-5
  source 1..18: mol_type = genomic DNA
aaaagcagaacctgagcg

SEQ ID NO: 89 - DNA, 18 bp, Homo sapiens
  misc_feature 1..18: note = Description of sequence: TCS-0080 HTT-6
  source 1..18: mol_type = genomic DNA
ccatcttggacccgtccc

SEQ ID NO: 90 - DNA, 18 bp, Homo sapiens
  misc_feature 1..18: note = Description of sequence: TCS-0081 HTT-7
  source 1..18: mol_type = genomic DNA
ggtaaaagcagaacctga

SEQ ID NO: 91 - DNA, 18 bp, Homo sapiens
  misc_feature 1..18: note = Description of sequence: TCS-0082 HTT-8
  source 1..18: mol_type = genomic DNA
ctgggccgcaggtaaaag

SEQ ID NO: 92 - DNA, 18 bp, Homo sapiens
  misc_feature 1..18: note = Description of sequence: TCS-0083 HTT-9
  source 1..18: mol_type = genomic DNA
atcttggacccgtcccgg

SEQ ID NO: 93 - DNA, 18 bp, Homo sapiens
  misc_feature 1..18: note = Description of sequence: TCS-0084 HTT-10
  source 1..18: mol_type = genomic DNA
gtccatcttggacccgtc

SEQ ID NO: 94 - DNA, 18 bp, Homo sapiens
  misc_feature 1..18: note = Description of sequence: TCS-0085 HTT-11
  source 1..18: mol_type = genomic DNA
gccgtccatcttggaccc

SEQ ID NO: 95 - DNA, 18 bp, Homo sapiens
  misc_feature 1..18: note = Description of sequence: TCS-0086 HTT-12
  source 1..18: mol_type = genomic DNA
agaacctgagcggccgtc

SEQ ID NO: 96 - DNA, 18 bp, Homo sapiens
  misc_feature 1..18: note = Description of sequence: TCS-0087 HTT-13
  source 1..18: mol_type = genomic DNA
gaatggggctctgggccg

SEQ ID NO: 97 - DNA, 18 bp, Homo sapiens
  misc_feature 1..18: note = Description of sequence: TCS-0088 HTT-14
  source 1..18: mol_type = genomic DNA
ggccgcaggtaaaagcag

SEQ ID NO: 98 - DNA, 18 bp, Homo sapiens
  misc_feature 1..18: note = Description of sequence: TCS-0089 HTT-15
  source 1..18: mol_type = genomic DNA
cctgagcggccgtccatc

SEQ ID NO: 99 - DNA, 18 bp, Homo sapiens
  misc_feature 1..18: note = Description of sequence: TCS-0090 HTT-16
  source 1..18: mol_type = genomic DNA
cggccgtccatcttggac

SEQ ID NO: 100 - DNA, 18 bp, Homo sapiens
  misc_feature 1..18: note = Description of sequence: TCS-0091 HTT-17
  source 1..18: mol_type = genomic DNA
ggacccgtcccggcagcc

SEQ ID NO: 101 - DNA, 18 bp, Homo sapiens
  misc_feature 1..18: note = Description of sequence: TCS-0092 SOD1-1
  source 1..18: mol_type = genomic DNA
cgcgactactttataggc

SEQ ID NO: 102 - DNA, 18 bp, Homo sapiens
  misc_feature 1..18: note = Description of sequence: TCS-0093 SOD1-2
  source 1..18: mol_type = genomic DNA
tccgcgactactttatag

SEQ ID NO: 103 - DNA, 18 bp, Homo sapiens
  misc_feature 1..18: note = Description of sequence: TCS-0094 SOD1-3
  source 1..18: mol_type = genomic DNA
ttataggccagacctccg

SEQ ID NO: 104 - DNA, 18 bp, Homo sapiens
  misc_feature 1..18: note = Description of sequence: TCS-0095 SOD1-4
  source 1..18: mol_type = genomic DNA
gtctccgcgactacttta

SEQ ID NO: 105 - DNA, 18 bp, Homo sapiens
  misc_feature 1..18: note = Description of sequence: TCS-0096 SOD1-5
  source 1..18: mol_type = genomic DNA
aggagactacgacgcaaa

SEQ ID NO: 106 - DNA, 18 bp, Homo sapiens
  misc_feature 1..18: note = Description of sequence: TCS-0097 SOD1-6
  source 1..18: mol_type = genomic DNA
ctgcaggagactacgacg

SEQ ID NO: 107 - DNA, 18 bp, Homo sapiens
  misc_feature 1..18: note = Description of sequence: TCS-0098 SOD1-7
  source 1..18: mol_type = genomic DNA
gagactacgacgcaaacc

SEQ ID NO: 108 - DNA, 18 bp, Homo sapiens
  misc_feature 1..18: note = Description of sequence: TCS-0099 SOD1-8
  source 1..18: mol_type = genomic DNA
gactacgacgcaaaccag

SEQ ID NO: 109 - DNA, 18 bp, Homo sapiens
  misc_feature 1..18: note = Description of sequence: TCS-0100 SOD1-10
  source 1..18: mol_type = genomic DNA
aggactgcaacggaaacc

SEQ ID NO: 110 - DNA, 18 bp, Homo sapiens
  misc_feature 1..18: note = Description of sequence: TCS-0101 SOD1-11
  source 1..18: mol_type = genomic DNA
cgactactttataggcca

SEQ ID NO: 111 - DNA, 18 bp, Homo sapiens
  misc_feature 1..18: note = Description of sequence: TCS-0102 SOD1-12
  source 1..18: mol_type = genomic DNA
ctgcaacggaaaccccag

SEQ ID NO: 112 - DNA, 18 bp, Homo sapiens
  misc_feature 1..18: note = Description of sequence: TCS-0103 SOD1-13
  source 1..18: mol_type = genomic DNA
acgacgcaaaccagcacc

SEQ ID NO: 113 - DNA, 18 bp, Homo sapiens
  misc_feature 1..18: note = Description of sequence: TCS-0104 SOD1-14
  source 1..18: mol_type = genomic DNA
gcaggagactacgacgca

SEQ ID NO: 114 - DNA, 18 bp, Homo sapiens
  misc_feature 1..18: note = Description of sequence: TCS-0105 SOD1-15
  source 1..18: mol_type = genomic DNA
gacgcaaaccagcacccc

SEQ ID NO: 115 - DNA, 18 bp, Homo sapiens
  misc_feature 1..18: note = Description of sequence: TCS-0106 SOD1-16
  source 1..18: mol_type = genomic DNA
ataggccagacctccgcg

SEQ ID NO: 116 - DNA, 18 bp, Homo sapiens
  misc_feature 1..18: note = Description of sequence: TCS-0107 SOD1-17
  source 1..18: mol_type = genomic DNA
cgcaaaccagcaccccgt

SEQ ID NO: 117 - DNA, 18 bp, Homo sapiens
  misc_feature 1..18: note = Description of sequence: TCS-0108 SOD1-19
  source 1..18: mol_type = genomic DNA
ctacgacgcaaaccagca

SEQ ID NO: 118 - DNA, 18 bp, Homo sapiens
  misc_feature 1..18: note = Description of sequence: TCS-0109 SOD1-20
  source 1..18: mol_type = genomic DNA
ccgtctccgcgactactt

SEQ ID NO: 119 - DNA, 18 bp, Homo sapiens
  misc_feature 1..18: note = Description of sequence: TCS-0110 SOD1-22
  source 1..18: mol_type = genomic DNA
cgctgcaggagactacga

SEQ ID NO: 120 - DNA, 18 bp, Homo sapiens
  misc_feature 1..18: note = Description of sequence: TCS-0111 SOD1-23
  source 1..18: mol_type = genomic DNA
gacgctgcaggagactac

SEQ ID NO: 121 - DNA, 36 bp, Homo sapiens
  misc_feature 1..36: note = Description of sequence: TCS-0112 MYC-1
  source 1..36: mol_type = genomic DNA
cgctggaattactacagcgacgctgcaggagactac

SEQ ID NO: 122 - DNA, 18 bp, Homo sapiens
  misc_feature 1..18: note = Description of sequence: TCS-0113 MYC-2
  source 1..18: mol_type = genomic DNA
actacagcgagttagata

SEQ ID NO: 123 - DNA, 18 bp, Homo sapiens
  misc_feature 1..18: note = Description of sequence: TCS-0114 MYC-3
  source 1..18: mol_type = genomic DNA
attactacagcgagttag

SEQ ID NO: 124 - DNA, 18 bp, Homo sapiens
  misc_feature 1..18: note = Description of sequence: TCS-0115 MYC-4
  source 1..18: mol_type = genomic DNA
ggaattactacagcgagt

SEQ ID NO: 125 - DNA, 18 bp, Homo sapiens
  misc_feature 1..18: note = Description of sequence: TCS-0116 MYC-5
  source 1..18: mol_type = genomic DNA
gcgagttagataaagccc

SEQ ID NO: 126 - DNA, 18 bp, Homo sapiens
  misc_feature 1..18: note = Description of sequence: TCS-0117 MYC-6
  source 1..18: mol_type = genomic DNA
tctcgctggaattactac

SEQ ID NO: 127 - DNA, 18 bp, Homo sapiens
  misc_feature 1..18: note = Description of sequence: TCS-0118 MYC-7
  source 1..18: mol_type = genomic DNA
acagcgagttagataaag

SEQ ID NO: 128 - DNA, 18 bp, Homo sapiens
  misc_feature 1..18: note = Description of sequence: TCS-0119 MYC-8
  source 1..18: mol_type = genomic DNA
ggctcttccaccctagcc

SEQ ID NO: 129 - DNA, 18 bp, Homo sapiens
  misc_feature 1..18: note = Description of sequence: TCS-0120 MYC-9
  source 1..18: mol_type = genomic DNA
tgcctctcgctggaatta

SEQ ID NO: 130 - DNA, 18 bp, Homo sapiens
  misc_feature 1..18: note = Description of sequence: TCS-0121 MYC-11
  source 1..18: mol_type = genomic DNA
cgcagcgcagctctgctc

SEQ ID NO: 131 - DNA, 18 bp, Homo sapiens
  misc_feature 1..18: note = Description of sequence: TCS-0122 MYC-12
  source 1..18: mol_type = genomic DNA
cctctcgctggaattact

SEQ ID NO: 132 - DNA, 18 bp, Homo sapiens
  misc_feature 1..18: note = Description of sequence: TCS-0123 MYC-13
  source 1..18: mol_type = genomic DNA
agctcgggggtcctcagc

SEQ ID NO: 133 - DNA, 18 bp, Homo sapiens
  misc_feature 1..18: note = Description of sequence: TCS-0124 MYC-14
  source 1..18: mol_type = genomic DNA
gccggggcccggcggtgg

SEQ ID NO: 134 - DNA, 18 bp, Homo sapiens
  misc_feature 1..18: note = Description of sequence: TCS-0125 MYC-15
  source 1..18: mol_type = genomic DNA
gcgagcagcacagctcgg

SEQ ID NO: 135 - DNA, 18 bp, Homo sapiens
  misc_feature 1..18: note = Description of sequence: TCS-0126 MYC-16
  source 1..18: mol_type = genomic DNA
ggcggccgcgagcagcac

SEQ ID NO: 136 - DNA, 18 bp, Homo sapiens
  misc_feature 1..18: note = Description of sequence: TCS-0127 MYC-17
  source 1..18: mol_type = genomic DNA
agcagcacagctcggggg

SEQ ID NO: 137 - DNA, 18 bp, Homo sapiens
  misc_feature 1..18: note = Description of sequence: TCS-0128 MYC-18
  source 1..18: mol_type = genomic DNA
gcacagctcgggggtcct

(19) United States
(12) Patent Application Publication
(10) Pub. No.: US 2025/0034574 A1
(43) Pub. Date: Jan. 30, 2025

(54) METHODS OF SILENCING EXPRESSION OF GENES AND USES THEREOF

(71) Applicant: UNIVERSITY OF MIAMI

(72) Inventors: Ramin SHIEKHATTAR, Miami, FL (US); Jingyin YUE, Miami, FL (US)

(21) Appl. No.: 18/716,340

(22) PCT Filed: Dec. 9, 2022
(86) PCT No.: PCT/US2022/081239

§ 371 Date: Jun. 4, 2024

Related U.S. Application Data
(60) Provisional application No. 63/288,149, filed on Dec. 10, 2021.

(51) Int. Cl. C12N 15/113; A61P 35/00

(52) U.S. Cl. CPC C12N 15/1135; A61P 35/00; C12N 15/1137; C12N 2310/11; C12N 2310/321; C12N 2310/341

(57) ABSTRACT

The present disclosure relates to methods of silencing expression of genes and uses thereof for treating diseases. Specifically, disclosed herein are Initiation of Transcription GAPmer (INTmers), wherein said INTmers comprise a nucleic acid molecule comprising 14-22 nucleotides, and further wherein said nucleic acid molecule comprises a central core of DNA flanked by RNA on each side.

### CROSS-REFERENCE TO RELATED APPLICATIONS

This application claims benefit of U.S. Provisional Application No. 63/288,149, filed Dec. 10, 2021, incorporated herein by reference in its entirety.

### STATEMENT REGARDING FEDERALLY SPONSORED RESEARCH

This invention was made with government support under Grant No. DP1CA228041 awarded by the National Institutes of Health. The government has certain rights in the invention.

### REFERENCE TO AN ELECTRONIC SEQUENCE LISTING

A Sequence Listing conforming to the rules of WIPO Standard ST.26 is hereby incorporated by reference. The Sequence Listing has been filed as an electronic document via EFS-Web in ASCII format encoded as XML. The electronic document, created on Dec. 9, 2022, is entitled “11348-015W01_ST26.xml”, and is 179,386 bytes in size.

### FIELD

The present disclosure relates to methods of silencing expression of genes and uses thereof for treating diseases.

### BACKGROUND

While it is predicted that only a fraction (˜3000) of the protein products of the human genome is subject to modulation by small molecules (the druggable genome), there are nearly 17,000 protein-coding genes whose protein products have been deemed undruggable. What is needed are novel systems and methods for modulating genes and their expression levels.

### SUMMARY

In some aspects, disclosed herein is a method of silencing expression of a gene of interest, the method comprising: a) exposing a cell comprising the gene of interest to an Initiation of Transcription GAPmer (INTmer), wherein said INTmer comprises a nucleic acid molecule comprising 14-22 nucleotides, and further wherein said nucleic acid molecule comprises a central core of DNA flanked by RNA on each side, wherein the central core of DNA is complementary to at least a portion of an mRNA transcript of the gene of interest; b) allowing the INTmer to hybridize to the nascent mRNA transcript of the gene of interest, wherein said INTmer hybridizes within the first 350 base pairs (for examples, the first 150 base pairs) of a 5′ end of the nascent mRNA transcript; and c) cleaving the nascent RNA:INTmer duplex, thereby silencing expression of the gene of interest.

In some embodiments, the gene of interest is a mutated gene. In some embodiments, the gene of interest is an oncogene. In some embodiments, the gene of interest is EGFR, BRAF, NRAS, APOB-100, Huntingtin (HTT), SOD1, or MYC.

In some embodiments, the cell is in vitro. In some embodiments, the cell is in vivo.

In some examples, the INTmer is 14, 15, 16, 17, 18, 19, 20, 21, or 22 nucleotides long. In some examples, the central core is 8, 9, 10, 11, or 12 nucleotides in length.

In some embodiments, the central core comprises at least one modified nucleotide. In some examples, said modified nucleotide comprises phosphorothioate and/or is 5-methylcytosine.

In some embodiments, each flanking RNA is 2, 3, 4, or 5 nucleotides in length on either side of the central core. In some embodiments, the RNA of the INTmer is modified. In some embodiments, the modified RNA comprises 2′-O-methoxy (MOE)-modified RNA or locked nucleic acid (LNA).

In some embodiments, the nascent mRNA transcript is about 50, 100, 150, or 200 nucleotides in length when the INTmer hybridizes with it. In some embodiments, the INTmer hybridizes within 5, 10, 15, 20, 25, 30, 35, 40, 45, 50, 55, 60, 65, 70, 75, 80, 85, 90, 95, 100, or 350 nucleotides or less of the CAP structure of the nascent RNA.

In some embodiments, the cleaved RNA transcript is de-CAPped. In some embodiments, the nascent RNA transcript is degraded by nuclease.

Also disclosed herein is a method of treating a subject with aberrant gene expression, the method comprising silencing expression of the aberrantly transcribed gene by: a) administering to the subject an Initiation of Transcription GAPmer (INTmer), wherein said INTmer comprises a nucleic acid molecule comprising 14-22 nucleotides, and further wherein said nucleic acid molecule comprises a central core of DNA flanked by RNA on each side, wherein the central core of DNA is complementary to at least a portion of an RNA transcript of the aberrantly transcribed gene; b) allowing the INTmer to hybridize to the nascent RNA transcript of the aberrantly transcribed gene, wherein said INTmer within the first 350 base pairs of a 5′ end of the nascent mRNA transcript; and c) allowing RNase H1 to cleave the nascent RNA:INTmer duplex, thereby silencing expression of the aberrantly transcribed gene.

In some embodiments, the subject has a chromosomal abnormality or the aberrant gene over-expressed.

In some embodiments, the INTmer is administered via a carrier (e.g., a nanoparticle, liposome, or exosome).

In some embodiments, the INTmer is conjugated to another molecule to facilitate delivery. In some embodiments, said molecule comprises LNPs, DPC™, TRiM™, or GalNAc.

Also, in some aspects, disclosed herein is method for identifying INTmers which are capable of downregulating gene expression of a gene of interest, the method comprising: a) sequencing at least the first 150 nucleotides of a region at a 5′ end of an RNA transcript of the gene of interest, or sequencing the gene of interest itself; b) creating a library of INTmers which are at least 80% complementary to region within the first 150 nucleotides of the RNA transcript of the gene of interest sequenced in step a), wherein said INTmers comprises a nucleic acid molecule comprising 16-20 nucleotides, and further wherein said nucleic acid molecule comprises a central core of DNA flanked by RNA on each side; c) exposing the INTmer library created in step b) to mammalian primary cells or cell lines expressing the nascent RNA transcript complementary to the INTmer sequencies; and d) determining which INTmers are capable of downregulation of gene expression of the gene of interest.

In some embodiments, the INTmer is 90% or more complementary to the RNA transcript.

In some embodiments, the method is carried out in cells in vitro. In some embodiments, the cells are cancer cells. In some embodiments, in step d), determining which INTmers are capable of downregulation of gene expression is carried out by measuring RNA levels of transcripts of the gene of interest.

In some embodiments, RT-qPCR is used to measure RNA levels. In some embodiments, protein levels of a protein corresponding to a gene of interest are measured (e.g., measured using Western Blotting).

In some embodiments, cellular viability is measured.

In some embodiments, the INTmer is designed against an RNA polymerase II pause-release domain.

In some embodiments, the pause-release domain corresponds to about nucleotides 40 to 350 of the nascent RNA transcript.

In some embodiments, the INTmer is designed against an RNA polymerase promoter-escape region. In some examples, the promoter-escape region corresponds to about nucleotides 20 to 80 of the nascent RNA transcript.

Also disclosed is composition comprising an INTmer, wherein the INTmer hybridizes to a CAGE region (the 5′-end of nascent RNA transcript marked by the CAP nucleotide, a guanine nucleotide connected to mRNA via an unusual 5′ to 5′ triphosphate linkage. This guanosine is methylated on the 7 position and is referred to as a 7-methylguanylate cap, abbreviated m7G), wherein said CAGE region comprises any of SEQ ID NOS: 1-9.

Further disclosed is a composition comprising an INTmer, wherein the INTmer comprises any of SEQ ID NOS: 10-137. In another embodiment, the INTmer can comprise at least 90% identity to any one SEQ ID NOS: 10-137. The INTmer can comprise a modification, such as the addition of α-tocopherol or cholesterol.

### DESCRIPTION OF DRAWINGS

FIG. 1 displays a graphic model showing mechanism of action for INTmers. Inside of the cell, INTmers hybridize with nascent transcripts of target gene and RNAase H1 dependent cleavage results in RNA de-capping and transcription termination of RNA.

FIGS. 2A-2B show CAGE data for BRAF and MYC shown in genome browser from HeLa cells (FIGS. 2A and 2B) and from adult lung tissue or lung cancer cells from DBTSS database (FIG. 2A).

FIGS. 3A-3D show EGFR INTmer screening. FIG. 3A shows schematic presentation showing the location of EGFR INTmers. FIG. 3B shows cell viability measured 4 days after ASO transfection. FIG. 3C shows immunoblots with whole cell lysate collected 2 days after ASO transfection (top) and quantification of EGFR protein level (bottom). FIG. 3D shows linear regression between EGFR protein level and cell viability.

FIGS. 4A-4C show analysis for specificity and mechanism of action of EGFR INTmers. FIG. 4A shows immunoblots with whole cell lysate collected 2 days after transfection of GAPmer or 2′-MOE RNA oligo. FIG. 4B shows immunoblots with whole cell lysate collected 2 days after siRNA transfection and 1 day after GAPmer transfection. FIG. 4C shows EGFR mRNA level measured with real-time PCR.

FIGS. 5A-5D show BRAF INTmer screening. FIG. 5A shows schematic presentation showing the location of BRAF INTmers. FIG. 5B shows cell viability measured 4 days after ASO transfection. FIG. 5C shows immunoblots with whole cell lysate collected 2 days after ASO transfection (Top) and quantification of BRAF protein level (Bottom). FIG. 5D shows linear regression between BRAF protein level and cell viability.

FIGS. 6A-6C show INTi-mediated silencing of EGFR induces rapid apoptotic cell death. Shown are immunoblots with whole cell lysate collected after transfection of EGFR-01 (FIG. 6A), treated with EGFR inhibitor WZ4002 (FIG. 6B), and transfection of wild-type or mutant EGFR-01 (FIG. 6C).

FIGS. 7A-7D show isoform specific targeting by INTmers. FIG. 7A shows immunoblots with whole cell lysate after 2 days ASO transfection (melanoma) or electroporation (melanocyte).

FIG. 7B shows cell viability measured 4 after ASO transfection or electroporation. FIGS. 7C-7D show BRAF RNA transcripts level measured with Real-time PCR in A375 (FIG. 7C) and melanocytes (FIG. 7D) at 8 hours after transfection. Specific primers were used to amplify BRAF mRNA, long isoform and all isoforms.

FIGS. 8A-8C show NRAS INTmer screening. FIG. 8A shows schematic presentation of NRAS INTmers location. FIG. 8B shows immunoblots with whole cell lysate 2 days after ASO transfection in MM047 cells. FIG. 8C shows cell viability measured 4 days after ASO transfection MM047 cells.

FIGS. 9A-9B show comparison between EGFR INTmer and Exon6 targeting ASOs. FIG. 9A shows immunoblots with whole cell lysate of H1975 cells collected 2 days after ASO transfection (100 nM). FIG. 9B shows cell survival curve measured 4 days after transfection and IC50 shown in table.

FIG. 10 shows apolipoprotein B-100 (ApoB-100) INTmer screening. Immunoblots with whole cell lysate of SNU449 cells collected 2 days after ASO transfection (200 nM).

FIGS. 11A-11B show Huntingtin (HTT) INTmer screening. FIG. 11A shows schematic presentation of HTT INTmers location. FIG. 11B Immunoblots with whole cell lysate 2 days after ASO transfection in lung cancer cell line H1437.

FIGS. 12A-12B show superoxide dismutase 1 (SOD1) INTmer screening. FIG. 12A shows schematic presentation showing the location of SOD1 INTmers. FIG. 12B shows immunoblots with whole cell lysate collected 2 days after ASO transfection (150 nM) in H1437 cells and quantification of immunoblots shown below.

FIGS. 13A-13C show MYC INTmer screening. FIG. 13A shows schematic presentation of MYC INTmers location. FIG. 13B shows cell viability measured 4 days after ASO transfection in of melanoma A375 cells. FIG. 13C shows immunoblots with whole cell lysate 2 days after ASO transfection in hepatocellular carcinoma cell line SNU449.

FIG. 14 shows schematic presentation showing the CAGE region used for INTmer design. The CAGE region for a gene is defined as chromatin sequence of 350 nucleotides in length, including 200 nucleotides of chromatin sequence with CAGE signals distributed in the center and immediately downstream chromatin sequence of 150 nucleotides.

FIG. 15A-C shows INTmer EGFR-1 treatment inhibits tumor progression in vivo. A, Tumor growth curve. Mice bearing H1975 xenografts were randomly grouped for treatment with Control-ASO or EGFR-1 at 25 mg/kg dosage 3 times per week for 10 times subcutaneously (s.c.). Tumor volume was measured 3 times per week and calculated by the formula length×width×width×0.5236. The shown were mean±SE. B, Mouse body weight during treatment. The body weight was measured 3 times per week and the shown were mean±SD. C, Shown were tumor weight measured at the endpoint of experiment (mean±SD). **, p<0.01 and **** p<0.0001.

### DETAILED DESCRIPTION

In some aspects, disclosed herein are methods of silencing expression of genes comprising using Initiation of Transcription GAPmers (INTmers) that are complementary to a portion (for example, the first 350 base pair of the 5′ end) of a nascent mRNA transcript transcribed from the gene. Also disclosed herein are compositions for silencing gene expression.

Reference will now be made in detail to the embodiments of the invention, examples of which are illustrated in the drawings and the examples. This invention may, however, be embodied in many different forms and should not be construed as limited to the embodiments set forth herein.

Unless defined otherwise, all technical and scientific terms used herein have the same meaning as commonly understood to one of ordinary skill in the art to which this disclosure belongs. The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments and are also disclosed. As used in this disclosure and in the appended claims, the singular forms “a”, “an”, “the”, include plural referents unless the context clearly dictates otherwise.

The following definitions are provided for the full understanding of terms used in this specification.

### Terminology

The term “about” as used herein when referring to a measurable value such as an amount, a percentage, and the like, is meant to encompass variations of ±20%, ±10%, ±5%, or ±1% from the measurable value.

“Administration” to a subject or “administering” includes any route of introducing or delivering to a subject an agent. Administration can be carried out by any suitable route, including oral, intravenous, intraperitoneal, intranasal, inhalation and the like. Administration includes self-administration and the administration by another.

The term “biocompatible” generally refers to a material and any metabolites or degradation products thereof that are generally non-toxic to the recipient and do not cause significant adverse effects to the subject.

The term “cancer” as used herein is defined as disease characterized by the rapid and uncontrolled growth of aberrant cells. Cancer cells can spread locally or through the bloodstream and lymphatic system to other parts of the body, Examples of various cancers include but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer and the like.

The term “cancerous cell”, as used herein, generally refers to any cells that exhibit, or are predisposed to exhibiting, unregulated growth. The term “cancer cells” and “tumor cells” are used interchangeably to refer to cells derived from a cancer or a tumor, or from a tumor cell line or a tumor cell culture.

“Composition” refers to any agent that has a beneficial biological effect. Beneficial biological effects include both therapeutic effects, e.g., treatment of a disorder or other undesirable physiological condition, and prophylactic effects, e.g., prevention of a disorder or other undesirable physiological condition. The terms also encompass pharmaceutically acceptable, pharmacologically active derivatives of beneficial agents specifically mentioned herein, including, but not limited to, a vector, polynucleotide, cells, salts, esters, amides, proagents, active metabolites, isomers, fragments, analogs, and the like. When the term “composition” is used, then, or when a particular composition is specifically identified, it is to be understood that the term includes the composition per se as well as pharmaceutically acceptable, pharmacologically active vector, polynucleotide, salts, esters, amides, proagents, conjugates, active metabolites, isomers, fragments, analogs, etc.

“Complementary” or “substantially complementary” refers to the hybridization or base pairing or the formation of a duplex between nucleotides or nucleic acids, such as, for instance, between the two strands of a double stranded DNA molecule or between an oligonucleotide primer and a primer binding site on a single stranded nucleic acid. Complementary nucleotides are, generally, A and T/U, or C and G. Two single-stranded RNA or DNA molecules are said to be substantially complementary when the nucleotides of one strand, optimally aligned and compared and with appropriate nucleotide insertions or deletions, pair with at least about 80% of the nucleotides of the other strand, usually at least about 90% to 95%, and more preferably from about 98 to 100%. Alternatively, substantial complementarity exists when an RNA or DNA strand will hybridize under selective hybridization conditions to its complement. Typically, selective hybridization will occur when there is at least about 65% complementary over a stretch of at least 14 to 25 nucleotides, at least about 75%, or at least about 90% complementary. See Kanehisa (1984) Nucl. Acids Res. 12:203.

A “control” is an alternative subject or sample used in an experiment for comparison purposes. A control can be “positive” or “negative.”

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom, Thus, a gene encodes a protein if transcription and translation of mRNA.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide.)

The “fragments,” whether attached to other sequences or not, can include insertions, deletions, substitutions, or other selected modifications of particular regions or specific amino acids residues, provided the activity of the fragment is not significantly altered or impaired compared to the nonmodified peptide or protein. These modifications can provide for some additional property, such as to remove or add amino acids capable of disulfide bonding, to increase its bio-longevity, to alter its secretory characteristics, etc. In any case, the fragment must possess a bioactive property, such as regulating the transcription of the target gene.

The term “gene” or “gene sequence” refers to the coding sequence or control sequence, or fragments thereof. A gene may include any combination of coding sequence and control sequence, or fragments thereof. Thus, a “gene” as referred to herein may be all or part of a native gene. A polynucleotide sequence as referred to herein may be used interchangeably with the term “gene”, or may include any coding sequence, non-coding sequence or control sequence, fragments thereof, and combinations thereof. The term “gene” or “gene sequence” includes, for example, control sequences upstream of the coding sequence (for example, the ribosome binding site).

The term “increased” or “increase” as used herein generally means an increase by a statically significant amount; for example, “increased” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

The term “reduced”, “reduce”, “reduction”, or “decrease” as used herein generally means a decrease by a statistically significant amount. However, for avoidance of doubt, “reduced” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (i.e. absent level as compared to a reference sample), or any decrease between 10-100% as compared to a reference level.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or higher identity over a specified region when compared and aligned for maximum correspondence over a comparison window or designated region) as measured using a BLAST or BLAST 2.0 sequence comparison algorithms with default parameters described below, or by manual alignment and visual inspection (see, e.g., NCBI web site or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 10 amino acids or 20 nucleotides in length, or more preferably over a region that is 10-50 amino acids or 20-50 nucleotides in length. As used herein, percent (%) nucleotide sequence identity is defined as the percentage of amino acids in a candidate sequence that are identical to the nucleotides in a reference sequence, after aligning the sequences and introducing gaps, if necessary, to achieve the maximum percent sequence identity. Alignment for purposes of determining percent sequence identity can be achieved in various ways that are within the skill in the art, for instance, using publicly available computer software such as BLAST, BLAST-2, ALIGN, ALIGN-2 or Megalign (DNASTAR) software. Appropriate parameters for measuring alignment, including any algorithms needed to achieve maximal alignment over the full-length of the sequences being compared can be determined by known methods.

For sequence comparisons, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

One example of an algorithm that is suitable for determining percent sequence identity and sequence similarity are the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al. (1990) J. Mol. Biol. 215:403-410). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a word length of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01.

“Inhibit”, “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

“Inhibitors” of expression or of activity are used to refer to inhibitory, activating, or modulating molecules, respectively, identified using in vitro and in vivo assays for expression or activity of a described target protein, e.g., antagonists and their homologs and mimetics. Inhibitors are agents that, e.g., inhibit expression or bind to, partially or totally block stimulation or activity, decrease, prevent, delay activation, inactivate, desensitize, or down regulate the activity of the described target protein, e.g., antagonists. Samples or assays comprising described target protein that are treated with a potential inhibitor are compared to control samples without the inhibitor to examine the extent of effect. Control samples (untreated with inhibitors) are assigned a relative activity value of 100%. Inhibition of a described target protein is achieved when the activity value relative to the control is about 80%, optionally 50% or 25, 10%, 5% or 1%.

As used herein, the term “level” refers to the amount of a target molecule in a sample, e.g., a sample from a subject. The amount of the molecule can be determined by any method known in the art and will depend in part on the nature of the molecule (i.e., gene, mRNA, cDNA, protein, enzyme, etc.). The art is familiar with quantification methods for nucleotides (e.g., genes, cDNA, mRNA, etc.) as well as proteins, polypeptides, enzymes, etc. It is understood that the amount or level of a molecule in a sample need not be determined in absolute terms, but can be determined in relative terms (e.g., when compared to a control or a sham or an untreated sample).

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

As used herein, the term “metastasis” is meant to refer to the process in which cancer cells originating in one organ or part of the body, with or without transit by a body fluid, and relocate to another part of the body and continue to replicate. Metastasized cells can subsequently form tumors which may further metastasize. Metastasis thus refers to the spread of cancer, from the part of the body where it originally occurred, to other parts of the body.

The term “nanoparticle” as used herein refers to a particle or structure which is biocompatible with and sufficiently resistant to chemical and/or physical destruction by the environment of such use so that a sufficient number of the nanoparticles remain substantially intact after delivery to the site of application or treatment and whose size is in the nanometer range. For the purposes of the present invention, a nanoparticle typically ranges from about 1 nm to about 1000 nm, preferably from about 50 nm to about 500 nm, more preferably from about 50 nm to about 350 nm.

The term “nucleic acid” as used herein means a polymer composed of nucleotides, e.g., deoxyribonucleotides (DNA) or ribonucleotides (RNA). The terms “ribonucleic acid” and “RNA” as used herein mean a polymer composed of ribonucleotides. The terms “deoxyribonucleic acid” and “DNA” as used herein mean a polymer composed of deoxyribonucleotides. (Used together with “polynucleotide” and “polypeptide”.)

The term “polypeptide” refers to a compound made up of a single chain of D- or L-amino acids or a mixture of D- and L-amino acids joined by peptide bonds.

The term “polynucleotide” refers to a single or double stranded polymer composed of nucleotide monomers.

“Pharmaceutically acceptable” component can refer to a component that is not biologically or otherwise undesirable, i.e., the component may be incorporated into a pharmaceutical formulation of the invention and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic, and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents.

As used herein, the term “carrier” encompasses any excipient, diluent, filler, salt, buffer, stabilizer, solubilizer, lipid, stabilizer, or other material well known in the art for use in pharmaceutical formulations. The choice of a carrier for use in a composition will depend upon the intended route of administration for the composition. The preparation of pharmaceutically acceptable carriers and formulations containing these materials is described in, e.g., Remington's Pharmaceutical Sciences, 21st Edition, ed. University of the Sciences in Philadelphia, Lippincott, Williams & Wilkins, Philadelphia, PA, 2005. Examples of physiologically acceptable carriers include saline, glycerol, DMSO, buffers such as phosphate buffers, citrate buffer, and buffers with other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™ (ICI, Inc.; Bridgewater, New Jersey), polyethylene glycol (PEG), and PLURONICS™ (BASF; Florham Park, NJ).

The term “primary tumor” refers to a tumor growing at the site of the cancer origin.

The term “polymer” as used herein refers to a relatively high molecular weight organic compound, natural or synthetic, whose structure can be represented by a repeated small unit, the monomer. Synthetic polymers are typically formed by addition or condensation polymerization of monomers. The polymer is suitable for use in the body of a subject, i.e. is biologically inert and physiologically acceptable, non-toxic, and is biodegradable in the environment of use, i.e. can be resorbed by the body. The term “polymer” encompasses all forms of polymers including, but not limited to, natural polymers, synthetic polymers, homopolymers, heteropolymers or copolymers, addition polymers, etc.

The term “copolymer” as used herein refers to a polymer formed from two or more different repeating units (monomer residues). Copolymer compasses all forms copolymers including, but not limited to block polymers, random copolymers, alternating copolymers, or graft copolymers. A “block copolymer” is a polymer formed from multiple sequences or blocks of the same monomer alternating in series with different monomer blocks. Block copolymers are classified according to the number of blocks they contain and how the blocks are arranged.

As used herein, the term “subject” or “host” can refer to living organisms such as mammals, including, but not limited to humans, livestock, dogs, cats, and other mammals. Administration of the therapeutic agents can be carried out at dosages and for periods of time effective for treatment of a subject. In some embodiments, the subject is a human.

As used herein, a “target”, “target molecule”, or “target cell” refers to a biomolecule or a cell that can be the focus of a therapeutic drug strategy, diagnostic assay, or a combination thereof, sometimes referred to as a theranostic. Therefore, a target can include, without limitation, many organic molecules that can be produced by a living organism or synthesized, for example, a protein or portion thereof, a peptide, a polysaccharide, an oligosaccharide, a sugar, a glycoprotein, a lipid, a phospholipid, a polynucleotide or portion thereof, an oligonucleotide, an aptamer, a nucleotide, a nucleoside, DNA, RNA, a DNA/RNA chimera, an antibody or fragment thereof, a receptor or a fragment thereof, a receptor ligand, a nucleic acid-protein fusion, a hapten, a nucleic acid, a virus or a portion thereof, an enzyme, a co-factor, a cytokine, a chemokine, as well as small molecules (e.g., a chemical compound), for example, primary metabolites, secondary metabolites, and other biological or chemical molecules that are capable of activating, inhibiting, or modulating a biochemical pathway or process, and/or any other affinity agent, among others.

“Therapeutic agent” refers to any composition that has a beneficial biological effect. Beneficial biological effects include both therapeutic effects, e.g., treatment of a disorder or other undesirable physiological condition, and prophylactic effects, e.g., prevention of a disorder or other undesirable physiological condition. The terms also encompass pharmaceutically acceptable, pharmacologically active derivatives of beneficial agents specifically mentioned herein, including, but not limited to, salts, esters, amides, proagents, active metabolites, isomers, fragments, analogs, and the like. When the terms “therapeutic agent” is used, then, or when a particular agent is specifically identified, it is to be understood that the term includes the agent per se as well as pharmaceutically acceptable, pharmacologically active salts, esters, amides, proagents, conjugates, active metabolites, isomers, fragments, analogs, etc.

The terms “treat,” “treating,” “treatment,” and grammatical variations thereof as used herein, include partially or completely delaying, alleviating, mitigating or reducing the intensity of one or more attendant symptoms of cancer or condition and/or alleviating, mitigating or impeding one or more symptoms of cancer. Treatments according to the invention may be applied preventively, prophylactically, palliatively or remedially. Prophylactic treatments are administered to a subject prior to onset (e.g., before obvious signs of cancer), during early onset (e.g., upon initial signs and symptoms of cancer), after an established development of cancer, or during prevention or mitigation of cancer relapse. Prophylactic administration can occur for several minutes to months prior to the manifestation of an infection.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

### Methods and Compositions Silencing Expression of Genes

In some aspects, disclosed herein is a method of silencing expression of a gene of interest, the method comprising: a) exposing a cell comprising the gene of interest to an Initiation of Transcription GAPmer (INTmer), wherein said INTmer comprises a nucleic acid molecule comprising 14-22 nucleotides, and further wherein said nucleic acid molecule comprises a central core of DNA flanked by RNA on each side, wherein the central core of DNA is complementary to at least a portion of an mRNA transcript of the gene of interest; b) allowing the INTmer to hybridize to the nascent mRNA transcript of the gene of interest, wherein said INTmer hybridizes within the first 350 base pairs (for examples, the first 150 base pairs) of a 5′ end of the nascent mRNA transcript; and c) cleaving the nascent RNA:INTmer duplex, thereby silencing expression of the gene of interest.

The term “INTmer” or “Initiation of Transcription GAPmer” refers to an antisense oligonucleotide targeted to an RNA sequence surrounding the start site of transcription, wherein the INTmer comprises a central core of DNA flanked by RNA on each side, wherein the central core of DNA is complementary to at least a portion of an mRNA transcript of the gene of interest.

In some embodiments, the INTmer hybridizes to the nascent mRNA transcript of the gene of interest within the first 350 base pairs (including, for example, the first 300 base pairs, first 250 base pairs, the first 200 base pairs, the first 150 base pairs, the first 100 base pairs, the first 50 base pairs, the first 45 base pairs, the first 40 base pairs, the first 35 base pairs, the first 30 base pairs, the first 25 base pairs, the first 20 base pairs, or the first 10 base pairs) of a 5′ end of the nascent mRNA transcript.

In some embodiments, the INTmer hybridizes to the nascent mRNA transcript of the gene of interest within nucleotide positions 1 to 300, nucleotide positions 1 to 100, nucleotide positions 1 to 50, nucleotide positions 5 to 250, nucleotide positions 5 to 200, nucleotide positions 5 to 150, nucleotide positions 5 to 100, nucleotide positions 10 to 150, nucleotide positions 10 to 100, nucleotide positions 10 to 80, nucleotide positions 10 to 50, nucleotide positions 20 to 200, nucleotide positions nucleotide positions 20 to 150, nucleotide positions 20 to 100, nucleotide positions 20 to 50, nucleotide positions 30 to 300, nucleotide positions 30 to 200, nucleotide positions 30 to 150, nucleotide positions 30 to 100, nucleotide positions 50 to 75, nucleotide positions 50 to 100, nucleotide positions 50 to 150, nucleotide positions 50 to 200, nucleotide positions 50 to 250, nucleotide positions 50 to 300, nucleotide positions 100 to 350 nucleotide, or positions 100 to 200 from a 5′ end of the nascent mRNA transcript.

It should be understood that the INTmer disclosed herein can hybridize within the first 350 base pairs of a 5′ end of the nascent mRNA transcript, including 200 base pairs where any single nucleotide can be marked by 7-methylguanylate (a CAP structure) followed by, for example, 150 nucleotides of nascent mRNA.

In some embodiments, the gene of interest is a mutated gene. In some embodiments, the gene of interest is related to a neurodegenerative disease. In some embodiments, the gene of interest is an oncogene. In some embodiments, the gene of interest comprises EGFR, BRAF, NRAS, APOB-100, Huntingtin (HTT), SOD1, BCL-2, MCL-1, or MYC. In some embodiments, the gene of interest is EGFR, BRAF, NRAS, APOB-100, HTT, SOD-1, or MYC.

The INTmer can be 5 to 100 nucleotides long (including, for example, 10 nucleotides long, 11 nucleotides long, 12 nucleotides long, 13 nucleotides long, 14 nucleotides long, 15 nucleotides long, 16 nucleotides long, 17 nucleotides long, 18 nucleotides long, 19 nucleotides long, 20 nucleotides long, 21 nucleotides long, 22 nucleotides long, 23 nucleotides long, 24 nucleotides long, 25 nucleotides long, 26 nucleotides long, 27 nucleotides long, 28 nucleotides long, 29 nucleotides long, 30 nucleotides long, 40 nucleotides long, 60 nucleotides long, 80 nucleotides long, or 100 nucleotides long). In some embodiments, the INTmer comprises 14-22 nucleotides.

The INTmer comprises central core of DNA flanked by RNA on each side. In some examples, the central core is 5, 6, 7, 8, 9, 10, 11, 12, 13, 14, 15, 16, 17, 18, 19, or 20 nucleotides in length. In some embodiments, the central core comprises at least one modified nucleotide. In some examples, the modified nucleotide comprises phosphorothioate and/or methylated at C5 position of cytosine. In some embodiments, each flanking RNA is 2, 3, 4, 5, 6, 7, 8, 9, or 10 nucleotides in length on either side of the central core. In some embodiments, the RNA of the INTmer is modified (for example, the modified RNA is a 2′-O-methoxy (MOE)-modified RNA and/or a locked nucleic acid (LNA).

The term “nascent mRNA transcript” herein refers to an RNA molecule in the process of being synthesized or a complete, newly synthesized RNA molecule before any alterations have been made (e.g., prior to nuclear processing or RNA editing. The nascent mRNA transcript can be from about 50 base pairs to millions of base pairs in length when the INTmer hybridizes with it. In some embodiments, the nascent mRNA transcript is about 50, 100, 150, or 200 nucleotides in length when the INTmer hybridizes with it. In some examples, the INTmer hybridizes within 5, 10, 15, 20, 25, 30, 35, 40, 45, 50, 55, 60, 65, 70, 75, 80, 85, 90, 95, 100, 150, 200, 250, or 350 nucleotides or less of the CAP structure of the nascent RNA.

INTmers that are complementary to start site sequences of the target gene form RNA-DNA hybrids, which are recognized by endonuclease and cleaved down-stream of start sites in nascent RNA. In some embodiments, the cleaved RNA transcript is de-CAPped. In some embodiments, the nascent RNA transcript is degraded by nuclease.

In some embodiments, the INTmer disclosed herein targets to the RNA sequences surrounding the start site of transcription as defined by cap analysis gene expression (CAGE) and extending beyond paused RNAPII.

Accordingly, in some embodiments, the INTmer disclosed herein targets the CAGE region or a portion thereof of the gene selected from EGFR, BRAF, NRAS, APOB-100, Huntingtin (HTT), SOD1, BCL-2, MCL-1, or MYC.

In some embodiments, the INTmer disclosed herein targets the CAGE region of gene EGFR or a portion thereof, wherein the CAGE region of gene EGFR comprises a sequence at least about 60% (for example, at least about 60%, at least about 65%, at least about 70%, at least about 75%, at least about 80%, at least about 85%, at least about 90%, at least about 95%, at least about 96%, at least about 97%, at least about 98%, or at least about 99%) identity to SEQ ID NO: 1.

In some embodiments, the INTmer targeting gene EGFR comprises a sequence at least about 60% (for example, at least about 60%, at least about 65%, at least about 70%, at least about 75%, at least about 80%, at least about 85%, at least about 90%, at least about 95%, at least about 96%, at least about 97%, at least about 98%, or at least about 99%) identity to SEQ ID NOs: 1-32 or a fragment thereof.

In some embodiments, the INTmer disclosed herein targets the CAGE region of gene BRAF or a portion thereof, wherein the CAGE region of gene BRAF comprises a sequence at least about 60% (for example, at least about 60%, at least about 65%, at least about 70%, at least about 75%, at least about 80%, at least about 85%, at least about 90%, at least about 95%, at least about 96%, at least about 97%, at least about 98%, or at least about 99%) identity to SEQ ID NO: 2 or 3.

In some embodiments, the INTmer targeting gene BRAF comprises a sequence at least about 60% (for example, at least about 60%, at least about 65%, at least about 70%, at least about 75%, at least about 80%, at least about 85%, at least about 90%, at least about 95%, at least about 96%, at least about 97%, at least about 98%, or at least about 99%) identity to SEQ ID NOs: 33-55 or a fragment thereof.

In some embodiments, the INTmer disclosed herein targets the CAGE region of gene NRAS or a portion thereof, wherein the CAGE region of gene NRAS comprises a sequence at least about 60% (for example, at least about 60%, at least about 65%, at least about 70%, at least about 75%, at least about 80%, at least about 85%, at least about 90%, at least about 95%, at least about 96%, at least about 97%, at least about 98%, or at least about 99%) identity to SEQ ID NO: 4.

In some embodiments, the INTmer targeting gene NRAS comprises a sequence at least about 60% (for example, at least about 60%, at least about 65%, at least about 70%, at least about 75%, at least about 80%, at least about 85%, at least about 90%, at least about 95%, at least about 96%, at least about 97%, at least about 98%, or at least about 99%) identity to SEQ ID NOs: 56-78 or a fragment thereof.

In some embodiments, the INTmer disclosed herein targets the CAGE region of gene APOB-100 or a portion thereof, wherein the CAGE region of gene APOB-100 comprises a sequence at least about 60% (for example, at least about 60%, at least about 65%, at least about 70%, at least about 75%, at least about 80%, at least about 85%, at least about 90%, at least about 95%, at least about 96%, at least about 97%, at least about 98%, or at least about 99%) identity to SEQ ID NO: 5.

In some embodiments, the INTmer targeting gene APOB-100 comprises a sequence at least about 60% (for example, at least about 60%, at least about 65%, at least about 70%, at least about 75%, at least about 80%, at least about 85%, at least about 90%, at least about 95%, at least about 96%, at least about 97%, at least about 98%, or at least about 99%) identity to SEQ ID NOs: 79-83 or a fragment thereof.

In some embodiments, the INTmer disclosed herein targets the CAGE region of gene HTT or a portion thereof, wherein the CAGE region of gene HTT comprises a sequence at least about 60% (for example, at least about 60%, at least about 65%, at least about 70%, at least about 75%, at least about 80%, at least about 85%, at least about 90%, at least about 95%, at least about 96%, at least about 97%, at least about 98%, or at least about 99%) identity to SEQ ID NO: 6.

In some embodiments, the INTmer targeting gene HTT comprises a sequence at least about 60% (for example, at least about 60%, at least about 65%, at least about 70%, at least about 75%, at least about 80%, at least about 85%, at least about 90%, at least about 95%, at least about 96%, at least about 97%, at least about 98%, or at least about 99%) identity to SEQ ID NOs: 84-100 or a fragment thereof.

In some embodiments, the INTmer disclosed herein targets the CAGE region of gene SOD1 or a portion thereof, wherein the CAGE region of gene SOD1 comprises a sequence at least about 60% (for example, at least about 60%, at least about 65%, at least about 70%, at least about 75%, at least about 80%, at least about 85%, at least about 90%, at least about 95%, at least about 96%, at least about 97%, at least about 98%, or at least about 99%) identity to SEQ ID NO: 7.

In some embodiments, the INTmer targeting gene SOD1 comprises a sequence at least about 60% (for example, at least about 60%, at least about 65%, at least about 70%, at least about 75%, at least about 80%, at least about 85%, at least about 90%, at least about 95%, at least about 96%, at least about 97%, at least about 98%, or at least about 99%) identity to SEQ ID NOs: 101-120 or a fragment thereof.

In some embodiments, the INTmer disclosed herein targets the CAGE region of gene MYC or a portion thereof, wherein the CAGE region of gene MYC comprises a sequence at least about 60% (for example, at least about 60%, at least about 65%, at least about 70%, at least about 75%, at least about 80%, at least about 85%, at least about 90%, at least about 95%, at least about 96%, at least about 97%, at least about 98%, or at least about 99%) identity to SEQ ID NO: 8 or 9.

In some embodiments, the INTmer targeting gene MYC comprises a sequence at least about 60% (for example, at least about 60%, at least about 65%, at least about 70%, at least about 75%, at least about 80%, at least about 85%, at least about 90%, at least about 95%, at least about 96%, at least about 97%, at least about 98%, or at least about 99%) identity to SEQ ID NOs: 121-137 or a fragment thereof.

In some embodiments, the INTmers are those provided in Table 2.

The INTmers disclosed herein can comprise one or more modified internucleoside linkages, modified sugar moieties and/or modified nucleobases. In some embodiments, oligonucleotides are chimeric oligonucleotides (e.g., chimeric oligomeric compounds). The terms “chimeric oligonucleotides” or “chimeras” are oligonucleotides that contain at least 2 chemically distinct regions (i.e., patterns and/or orientations of motifs of chemically modified subunits arranged along the length of the oligonucleotide) each made up of at least one monomer unit, i.e., a nucleotide or nucleoside in the case of a nucleic acid based oligonucleotide compound. Representative United States patents that teach the preparation of such chimeric oligonucleotide structures include, but are not limited to, U.S. Pat. Nos. 5,013,830; 5,149,797; 5,220,007; 5,256,775; 5,366,878; 5,403,711; 5,491,133; 5,565,350; 5,623,065; 5,652,355; 5,652,356; and 5,700,922, each of which is herein incorporated by reference in its entirety.

The INTmers disclosed herein can also be modified to have one or more stabilizing groups. In some embodiments, the stabilizing groups are attached to one or both termini of oligonucleotides to enhance properties such as nuclease stability. In some embodiments, the stabilizing groups are cap structures. By “cap structure or terminal cap moiety” is meant chemical modifications, which have been incorporated at either terminus of oligonucleotides (see for example WO 97/26270, which is herein incorporated by reference in its entirety). These terminal modifications may serve to protect the oligonucleotides having terminal nucleic acid molecules from exonuclease degradation and/or may help in the delivery and/or localization of the oligonucleotide within a cell. The oligonucleotide may contain the cap at the 5′-terminus (5′-cap), the 3′-terminus (3′-cap), or both the 5′-terminus and the 3′-termini. In the case of double-stranded oligonucleotides, the cap may be present at either or both termini of either strand. Cap structures are known in the art and include, for example, inverted deoxy abasic caps. Further 3′ and 5′-stabilizing groups that can be used to cap one or both ends of an oligonucleotide (e.g., antisense) compound to impart nuclease stability include those disclosed in WO 03/004602, which is herein incorporated by reference in its entirety.

In some embodiments, the 5′-cap of an INTmer can include a structure that is an inverted abasic residue (moiety), 4′,5′-methylene nucleotide; 1-(beta-D-erythrofuranosyl) nucleotide, 4′-thio nucleotide, carbocyclic nucleotide; 1,5-anhydrohexitol nucleotide; L-nucleotides; alpha-nucleotides; modified base nucleotide; phosphorodithioate linkage; threo-pentofuranosyl nucleotide; acyclic 3′,4′-seco nucleotide; acyclic 3,4-dihydroxybutyl nucleotide; acyclic 3,5-dihydroxypentyl nucleotide, 3′-3′-inverted nucleotide moiety; 3′-3′-inverted abasic moiety; 3′-2′-inverted nucleotide moiety; 3′-2′-inverted abasic moiety; 1,4-butanediol phosphate; 3′-phosphoramidate; hexylphosphate; aminohexyl phosphate; 3′-phosphate; 3′-phosphorothioate; phosphorodithioate; or bridging or non-bridging methylphosphonate moiety (see e.g., WO 97/26270, which is herein incorporated by reference in its entirety).

In some embodiments, the 3′-cap of an INTmer includes for example a 4′,5′-methylene nucleotide; 1-(beta-D-erythrofuranosyl) nucleotide; 4′-thio nucleotide, carbocyclic nucleotide; 5′-amino-alkyl phosphate; 1,3-diamino-2-propyl phosphate, 3-aminopropyl phosphate; 6-aminohexyl phosphate; 1,2-aminododecyl phosphate; hydroxypropyl phosphate; 1,5-anhydrohexitol nucleotide; L-nucleotide; alpha-nucleotide; modified base nucleotide; phosphorodithioate; threo-pentofuranosyl nucleotide; acyclic 3′,4′-seco nucleotide; 3,4-dihydroxybutyl nucleotide; 3,5-dihydroxypentyl nucleotide, 5′-5′-inverted nucleotide moiety; 5′-5′-inverted abasic moiety; 5′-phosphoramidate; 5′-phosphorothioate; 1,4-butanediol phosphate; 5′-amino; bridging and/or non-bridging 5′-phosphoramidate, phosphorothioate and/or phosphorodithioate, bridging or non-bridging methylphosphonate and 5′-mercapto moieties (see also the stabilizing groups disclosed in Beaucage et al., 1993, Tetrahedron 49:1925; which is herein incorporated by reference in its entirety).

In one embodiment, modified INTmers are prepared by covalently attaching conjugate groups to functional groups such as hydroxyl or amino groups. Useful conjugate groups include, but are not limited to, intercalators, reporter molecules, polyamines, polyamides, polyethylene glycols, polyethers, and groups that enhance the pharmacodynamic or pharmacokinetic properties of the INTmer. Typical conjugate groups include cholesterols, carbohydrates, biotin, phenazine, folate, phenanthridine and anthraquinone. Representative conjugate groups are disclosed in WO/1993/007883 and U.S. Pat. No. 6,287,860, each of which is herein incorporated by reference in its entirety. In a preferred embodiment, the modification to the INTmer comprises α-tocopherol. Examples of using α-tocopherol with a DNA/RNA heteroduplex are known in the art, and are hereby incorporated by reference in their entirety (Nagata, T., Dwyer, C. A., Yoshida-Tanaka, K. et al. Cholesterol-functionalized DNA/RNA heteroduplexes cross the blood-brain barrier and knock down genes in the rodent CNS. Nat Biotechnol (2021); Nishina, K., Piao, W., Yoshida-Tanaka, K. et al. DNA/RNA heteroduplex oligonucleotide for highly efficient gene silencing. Nat Commun 6, 7969 (2015); Yokota, T. Novel oligonucleotide based on DNA/RNA heteroduplex structures: Opening a new horizon for human gene therapy. Cutting-Edge Research at TMDU.)

Conjugate groups can be attached to various positions of an oligonucleotide directly or via an optional linking group. The term linking group is intended to include all groups amenable to attachment of a conjugate group to an oligomeric compound. Linking groups are bivalent groups useful for attachment of chemical functional groups, conjugate groups, reporter groups and other groups to selective sites in a parent compound such as for example an oligomeric compound. In general a bifunctional linking moiety comprises a hydrocarbyl moiety having two functional groups. One of the functional groups is selected to bind to a parent molecule or compound of interest and the other is selected to bind essentially any selected group such as chemical functional group or a conjugate group. In some embodiments, the linker comprises a chain structure or an oligomer of repeating units such as ethylene glycol or amino acid units. Examples of functional groups that are routinely used in bifunctional linking moieties include, but are not limited to, electrophiles for reacting with nucleophilic groups and nucleophiles for reacting with electrophilic groups. In some embodiments, bifunctional linking moieties include amino, hydroxyl, carboxylic acid, thiol, unsaturations (e.g., double or triple bonds), and the like. Some nonlimiting examples of bifunctional linking moieties include 8-amino-3,6-dioxaoctanoic acid (ADO), succinimidyl 4-(N-maleimidomethyl)cyclohexane-1-carboxylate (SMCC) and 6-aminohexanoic acid (AHEX or AHA). Other linking groups include, but are not limited to, substituted C1-C10 alkyl, substituted or unsubstituted C2-C10 alkenyl or substituted or unsubstituted C2-C10 alkynyl, wherein a nonlimiting list of preferred substituent groups includes hydroxyl, amino, alkoxy, carboxy, benzyl, phenyl, nitro, thiol, thioalkoxy, halogen, alkyl, aryl, alkenyl and alkynyl. Further representative linking groups are disclosed for example in WO 94/01550 and WO 94/01550.

In some aspects, disclosed herein is a method of treating a subject with aberrant gene expression, the method comprising silencing expression of the aberrantly transcribed gene by: a) administering to the subject an Initiation of Transcription GAPmer (INTmer), wherein said INTmer comprises a nucleic acid molecule comprising 14-22 nucleotides, and further wherein said nucleic acid molecule comprises a central core of DNA flanked by RNA on each side, wherein the central core of DNA is complementary to at least a portion of an RNA transcript of the aberrantly transcribed gene; b) allowing the INTmer to hybridize to the nascent RNA transcript of the aberrantly transcribed gene, wherein said INTmer within the first 150 base pairs of a 5′ end of the nascent mRNA transcript; and c) allowing RNase H1 to cleave the nascent RNA:INTmer duplex, thereby silencing expression of the aberrantly transcribed gene.

In some embodiments, the aberrantly transcribed gene is a mutated gene or is aberrantly over-expressed. In some examples, the aberrantly transcribed gene comprises an activating mutation. In some examples, the mutated gene is an oncogene. In some embodiments, the gene of interest comprises EGFR, BRAF, NRAS, APOB-100, Huntingtin (HTT), SOD1, BCL-2, MCL-1, or MYC. In some embodiments, the gene of interest is EGFR, BRAF, NRAS, APOB-100, HTT, SOD-1, or MYC.

In some embodiments, the subject has a chromosomal abnormality or the aberrant gene over-expressed.

The INTmer can be administered via a carrier (for examples, a pharmaceutically acceptable carrier. In some embodiments, the carrier is a nanoparticle, liposome, or exosome. The nanoparticles described herein include one or more moieties that target the nanoparticles to a cancer cell.

The nanoparticle used herein can be any nanoparticle useful for the delivery of nucleic acids and/or polypeptides. The term “nanoparticle” as used herein refers to a particle or structure which is biocompatible with and sufficiently resistant to chemical and/or physical destruction by the environment of such use so that a sufficient number of the nanoparticles remain substantially intact after delivery to the site of application or treatment and whose size is in the nanometer range. In some embodiments, the nanoparticle comprises a lipid-like nanoparticle. See, for example, WO/2016/187531A1, WO/2017/176974, WO/2019/027999, or Li, B et al. An Orthogonal array optimization of lipid-like nanoparticles for mRNA delivery in vivo. Nano Lett. 2015, 15, 8099-8107; which are incorporated herein by reference in their entireties. In some embodiments, the nanoparticle is a porous silica nanoparticle (pSi). In some embodiments, the nanoparticle comprises poly (lactide-co-glycolide) (PLGA). Porous silica nanoparticles are well known in the art. See, for example, U.S. Pat. No. 10,143,660; US Application Publication No. 2013/0216807; International Publication No. 2013/056132; which are incorporated herein by reference in their entireties.

Nanoparticles disclosed herein include one, two, three or more biocompatible and/or biodegradable polymers. For example, a contemplated nanoparticle may include about 10 to about 99 weight percent of a one or more block co-polymers that include a biodegradable polymer and polyethylene glycol, and about 0 to about 50 weight percent of a biodegradable homopolymer. Polymers can include, for example, both biostable and biodegradable polymers, such as microcrystalline cellulose, hydroxypropyl cellulose, hydroxypropyl methylcellulose, polyalkylene oxides such as polyethylene oxide (PEG), polyanhydrides, poly(ester anhydrides), polyhydroxy acids such as polylactide (PLA), polyglycolide (PGA), poly(lactide-co-glycolide) (PLGA), poly-3-hydroxybutyrate (PHB), poly-ethyleneimine (PEI), polyvinyl alcohol (PVA) and copolymers thereof, poly-4-hydroxybutyrate (P4HB) and copolymers thereof, polycaprolactone and copolymers thereof, and combinations thereof.

The term “exosome”, as used herein, refers to a cell-derived membranous vesicle. They refer to extracellular vesicles, which are generally of between 30 and 200 nm, for example in the range of 50-100 nm in size. In some aspects, the extracellular vesicles can be in the range of 20-300 nm in size, for example 30-250 nm in size, for example 50-200 nm in size. In some aspects, the extracellular vesicles are defined by a lipidic bilayer membrane. Exosomes are released from most cell types and can be found in many biological fluids.

In some embodiments, the nanoparticle, liposome, or exosome has a diameter from about 1 nm to about 1000 nm. In some embodiments, the nanoparticle, liposome, or exosome has a diameter less than, for example, about 1000 nm, about 950 nm, about 900 nm, about 850 nm, about 800 nm, about 750 nm, about 700 nm, about 650 nm, about 600 nm, about 550 nm, about 500 nm, about 450 nm, about 400 nm, about 350 nm, about 300 nm, about 290 nm, about 280 nm, about 270 nm, about 260 nm, about 250 nm, about 240 nm, about 230 nm, about 220 nm, about 210 nm, about 200 nm, about 190 nm, about 180 nm, about 170 nm, about 160 nm, about 150 nm, about 140 nm, about 130 nm, about 120 nm, about 110 nm, about 100 nm, about 90 nm, about 80 nm, about 70 nm, about 60 nm, about 50 nm, about 40 nm, about 30 nm, about 20 nm, or about 10 nm. In some embodiments, the nanoparticle has a diameter, for example, from about 20 nm to about 1000 nm, from about 20 nm to about 800 nm, from about 20 nm to about 700 nm, from about 30 nm to about 600 nm, from about 30 nm to about 500 nm, from about 40 nm to about 400 nm, from about 40 nm to about 300 nm, from about 40 nm to about 250 nm, from about 50 nm to about 250 nm, from about 50 nm to about 200 nm, from about 50 nm to about 150 nm, from about 60 nm to about 150 nm, from about 70 nm to about 150 nm, from about 80 nm to about 150 nm, from about 90 nm to about 150 nm, from about 100 nm to about 150 nm, from about 110 nm to about 150 nm, from about 120 nm to about 150 nm, from about 90 nm to about 140 nm, from about 90 nm to about 130 nm, from about 90 nm to about 120 nm, from 100 nm to about 140 nm, from about 100 nm to about 130 nm, from about 100 nm to about 120 nm, from about 100 nm to about 110 nm, from about 110 nm to about 120 nm, from about 110 nm to about 130 nm, from about 110 nm to about 140 nm, from about 90 nm to about 200 nm, from about 100 nm to about 195 nm, from about 110 nm to about 190 nm, from about 120 nm to about 185 nm, from about 130 nm to about 180 nm, from about 140 nm to about 175 nm, from 150 nm to 175 nm, or from about 150 nm to about 170 nm. In some embodiments, the nanoparticle, liposome, or exosome has a diameter from about 50 nm to about 100 nm. In some embodiments, the nanoparticle, liposome, or exosome has a diameter from about 100 nm to about 250 nm. In some embodiments, the nanoparticle, liposome, or exosome has a diameter from about 150 nm to about 175 nm. In some embodiments, the nanoparticle, liposome, or exosome has a diameter from about 135 nm to about 175 nm. The particles can have any shape but are generally spherical in shape.

Any suitable synthetic or natural biocompatible polymers may be used. Such polymers are recognizable and identifiable by one or ordinary skill in the art. The polymers used for nanoparticles are known in the art. See, e.g., U.S. Patent Publication NO: US20170216219A1, incorporated by reference herein in its entirety. In some embodiments, the nanoparticle comprise poly(lactic-co-glycolic acid) (PLGA)-block(b)-polyethylene glycol (PEG).

The amount of a therapeutic agent that can be present in the nanoparticle, liposome, or exosome can be from about 0.1% to about 90% of its particle weight. For example, the amount of a therapeutic agent present in the nanoparticle, liposome, or exosome can be from about 0.1%, about 0.2%, about 0.3%, about 0.4%, about 0.5%, about 0.6%, about 0.7%, about 0.8%, about 0.9%, about 1%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, about 10%, about 10.5%, about 11%, about 11.5%, about 12%, about 12.5%, about 13%, about 13.5%, about 14%, about 15%, about 16%, about 17%, about 18%, about 19%, about 20%, about 22%, about 24%, about 26%, about 28%, about 30%, about 32%, about 34%, about 36%, about 38%, about 40%, about 42%, about 44%, about 46%, about 50%, about 55%, about 60%, about 65%, about 70%, about 75%, or about 80% of its particle weight.

Also in some embodiments, the INTmer is conjugated to another molecule to facilitate delivery. In some embodiments, said molecule comprises LNPs, DPC™, TRiM™, or GalNAc. Other delivery methods are disclosed in Hu, B., Zhong, L., Weng, Y. et al. Therapeutic siRNA: state of the art. Sig Transduct Target Ther 5, 101 (2020), herein incorporated by reference in its entirety.

A cancer can be selected from, but is not limited to, a hematologic cancer, lymphoma, colorectal cancer, colon cancer, lung cancer, a head and neck cancer, ovarian cancer, prostate cancer, testicular cancer, renal cancer, skin cancer, cervical cancer, pancreatic cancer, and breast cancer. In one aspect, the cancer comprises a solid tumor. In another aspect, the cancer is selected from acute myeloid leukemia, myelodysplastic syndrome, chronic myeloid leukemia, acute lymphoblastic leukemia, myelofibrosis, multiple myeloma. In another aspect, the cancer is selected from a leukemia, a lymphoma, a sarcoma, a carcinoma and may originate in the marrow, brain, lung, breast, pancreas, liver, head and neck, skin, reproductive tract, prostate, colon, liver, kidney, intraperitoneum, bone, joint, and eye.

It is intended herein that the disclosed methods of inhibiting, reducing, and/or preventing cancer metastasis and/or recurrence can additionally comprise combination therapy, such that administration of any anti-cancer agent known in the art can also be administered to the subject. Examples of anti-cancer agents that can be used in said combination therapy include, but is not limited to Abemaciclib, Abiraterone Acetate, Abitrexate (Methotrexate), Abraxane (Paclitaxel Albumin-stabilized Nanoparticle Formulation), ABVD, ABVE, ABVE-PC, AC, AC-T, Adcetris (Brentuximab Vedotin), ADE, Ado-Trastuzumab Emtansine, Adriamycin (Doxorubicin Hydrochloride), Afatinib Dimaleate, Afinitor (Everolimus), Akynzeo (Netupitant and Palonosetron Hydrochloride), Aldara (Imiquimod), Aldesleukin, Alecensa (Alectinib), Alectinib, Alemtuzumab, Alimta (Pemetrexed Disodium), Aliqopa (Copanlisib Hydrochloride), Alkeran for Injection (Melphalan Hydrochloride), Alkeran Tablets (Melphalan), Aloxi (Palonosetron Hydrochloride), Alunbrig (Brigatinib), Ambochlorin (Chlorambucil), Amboclorin Chlorambucil), Amifostine, Aminolevulinic Acid, Anastrozole, Aprepitant, Aredia (Pamidronate Disodium), Arimidex (Anastrozole), Aromasin (Exemestane), Arranon (Nelarabine), Arsenic Trioxide, Arzerra (Ofatumumab), Asparaginase Erwinia chrysanthemi, Atezolizumab, Avastin (Bevacizumab), Avelumab, Axitinib, Azacitidine, Bavencio (Avelumab), BEACOPP, Becenum (Carmustine), Beleodaq (Belinostat), Belinostat, Bendamustine Hydrochloride, BEP, Besponsa (Inotuzumab Ozogamicin), Bevacizumab, Bexarotene, Bexxar (Tositumomab and Iodine I 131 Tositumomab), Bicalutamide, BiCNU (Carmustine), Bleomycin, Blinatumomab, Blincyto (Blinatumomab), Bortezomib, Bosulif (Bosutinib), Bosutinib, Brentuximab Vedotin, Brigatinib, BuMel, Busulfan, Busulfex (Busulfan), Cabazitaxel, Cabometyx (Cabozantinib-S-Malate), Cabozantinib-S-Malate, CAF, Campath (Alemtuzumab), Camptosar, (Irinotecan Hydrochloride), Capecitabine, CAPOX, Carac (Fluorouracil—Topical), Carboplatin, CARBOPLATIN-TAXOL, Carfilzomib, Carmubris (Carmustine), Carmustine, Carmustine Implant, Casodex (Bicalutamide), CEM, Ceritinib, Cerubidine (Daunorubicin Hydrochloride), Cervarix (Recombinant HPV Bivalent Vaccine), Cetuximab, CEV, Chlorambucil, CHLORAMBUCIL-PREDNISONE, CHOP, Cisplatin, Cladribine, Clafen (Cyclophosphamide), Clofarabine, Clofarex (Clofarabine), Clolar (Clofarabine), CMF, Cobimetinib, Cometriq (Cabozantinib-S-Malate), Copanlisib Hydrochloride, COPDAC, COPP, COPP-ABV, Cosmegen (Dactinomycin), Cotellic (Cobimetinib), Crizotinib, CVP, Cyclophosphamide, Cyfos (Ifosfamide), Cyramza (Ramucirumab), Cytarabine, Cytarabine Liposome, Cytosar-U (Cytarabine), Cytoxan (Cyclophosphamide), Dabrafenib, Dacarbazine, Dacogen (Decitabine), Dactinomycin, Daratumumab, Darzalex (Daratumumab), Dasatinib, Daunorubicin Hydrochloride, Daunorubicin Hydrochloride and Cytarabine Liposome, Decitabine, Defibrotide Sodium, Defitelio (Defibrotide Sodium), Degarelix, Denileukin Diftitox, Denosumab, DepoCyt (Cytarabine Liposome), Dexamethasone, Dexrazoxane Hydrochloride, Dinutuximab, Docetaxel, Doxil (Doxorubicin Hydrochloride Liposome), Doxorubicin Hydrochloride, Doxorubicin Hydrochloride Liposome, Dox-SL (Doxorubicin Hydrochloride Liposome), DTIC-Dome (Dacarbazine), Durvalumab, Efudex (Fluorouracil—Topical), Elitek (Rasburicase), Ellence (Epirubicin Hydrochloride), Elotuzumab, Eloxatin (Oxaliplatin), Eltrombopag Olamine, Emend (Aprepitant), Empliciti (Elotuzumab), Enasidenib Mesylate, Enzalutamide, Epirubicin Hydrochloride, EPOCH, Erbitux (Cetuximab), Eribulin Mesylate, Erivedge (Vismodegib), Erlotinib Hydrochloride, Erwinaze (Asparaginase Erwinia chrysanthemi), Ethyol (Amifostine), Etopophos (Etoposide Phosphate), Etoposide, Etoposide Phosphate, Evacet (Doxorubicin Hydrochloride Liposome), Everolimus, Evista, (Raloxifene Hydrochloride), Evomela (Melphalan Hydrochloride), Exemestane, 5-FU (Fluorouracil Injection), 5-FU (Fluorouracil—Topical), Fareston (Toremifene), Farydak (Panobinostat), Faslodex (Fulvestrant), FEC, Femara (Letrozole), Filgrastim, Fludara (Fludarabine Phosphate), Fludarabine Phosphate, Fluoroplex (Fluorouracil—Topical), Fluorouracil Injection, Fluorouracil—Topical, Flutamide, Folex (Methotrexate), Folex PFS (Methotrexate), FOLFIRI, FOLFIRI-BEVACIZUMAB, FOLFIRI-CETUXIMAB, FOLFIRINOX, FOLFOX, Folotyn (Pralatrexate), FU-LV, Fulvestrant, Gardasil (Recombinant HPV Quadrivalent Vaccine), Gardasil 9 (Recombinant HPV Nonavalent Vaccine), Gazyva (Obinutuzumab), Gefitinib, Gemcitabine Hydrochloride, GEMCITABINE-CISPLATIN, GEMCITABINE-OXALIPLATIN, Gemtuzumab Ozogamicin, Gemzar (Gemcitabine Hydrochloride), Gilotrif (Afatinib Dimaleate), Gleevec (Imatinib Mesylate), Gliadel (Carmustine Implant), Gliadel wafer (Carmustine Implant), Glucarpidase, Goserelin Acetate, Halaven (Eribulin Mesylate), Hemangeol (Propranolol Hydrochloride), Herceptin (Trastuzumab), HPV Bivalent Vaccine, Recombinant, HPV Nonavalent Vaccine, Recombinant, HPV Quadrivalent Vaccine, Recombinant, Hycamtin (Topotecan Hydrochloride), Hydrea (Hydroxyurea), Hydroxyurea, Hyper-CVAD, Ibrance (Palbociclib), Ibritumomab Tiuxetan, Ibrutinib, ICE, Iclusig (Ponatinib Hydrochloride), Idamycin (Idarubicin Hydrochloride), Idarubicin Hydrochloride, Idelalisib, Idhifa (Enasidenib Mesylate), Ifex (Ifosfamide), Ifosfamide, Ifosfamidum (Ifosfamide), IL-2 (Aldesleukin), Imatinib Mesylate, Imbruvica (Ibrutinib), Imfinzi (Durvalumab), Imiquimod, Imlygic (Talimogene Laherparepvec), Inlyta (Axitinib), Inotuzumab Ozogamicin, Interferon Alfa-2b, Recombinant, Interleukin-2 (Aldesleukin), Intron A (Recombinant Interferon Alfa-2b), Iodine 1131 Tositumomab and Tositumomab, Ipilimumab, Iressa (Gefitinib), Irinotecan Hydrochloride, Irinotecan Hydrochloride Liposome, Istodax (Romidepsin), Ixabepilone, Ixazomib Citrate, Ixempra (Ixabepilone), Jakafi (Ruxolitinib Phosphate), JEB, Jevtana (Cabazitaxel), Kadcyla (Ado-Trastuzumab Emtansine), Keoxifene (Raloxifene Hydrochloride), Kepivance (Palifermin), Keytruda (Pembrolizumab), Kisqali (Ribociclib), Kymriah (Tisagenlecleucel), Kyprolis (Carfilzomib), Lanreotide Acetate, Lapatinib Ditosylate, Lartruvo (Olaratumab), Lenalidomide, Lenvatinib Mesylate, Lenvima (Lenvatinib Mesylate), Letrozole, Leucovorin Calcium, Leukeran (Chlorambucil), Leuprolide Acetate, Leustatin (Cladribine), Levulan (Aminolevulinic Acid), Linfolizin (Chlorambucil), LipoDox (Doxorubicin Hydrochloride Liposome), Lomustine, Lonsurf (Trifluridine and Tipiracil Hydrochloride), Lupron (Leuprolide Acetate), Lupron Depot (Leuprolide Acetate), Lupron Depot-Ped (Leuprolide Acetate), Lynparza (Olaparib), Marqibo (Vincristine Sulfate Liposome), Matulane (Procarbazine Hydrochloride), Mechlorethamine Hydrochloride, Megestrol Acetate, Mekinist (Trametinib), Melphalan, Melphalan Hydrochloride, Mercaptopurine, Mesna, Mesnex (Mesna), Methazolastone (Temozolomide), Methotrexate, Methotrexate LPF (Methotrexate), Methylnaltrexone Bromide, Mexate (Methotrexate), Mexate-AQ (Methotrexate), Midostaurin, Mitomycin C, Mitoxantrone Hydrochloride, Mitozytrex (Mitomycin C), MOPP, Mozobil (Plerixafor), Mustargen (Mechlorethamine Hydrochloride), Mutamycin (Mitomycin C), Myleran (Busulfan), Mylosar (Azacitidine), Mylotarg (Gemtuzumab Ozogamicin), Nanoparticle Paclitaxel (Paclitaxel Albumin-stabilized Nanoparticle Formulation), Navelbine (Vinorelbine Tartrate), Necitumumab, Nelarabine, Neosar (Cyclophosphamide), Neratinib Maleate, Nerlynx (Neratinib Maleate), Netupitant and Palonosetron Hydrochloride, Neulasta (Pegfilgrastim), Neupogen (Filgrastim), Nexavar (Sorafenib Tosylate), Nilandron (Nilutamide), Nilotinib, Nilutamide, Ninlaro (Ixazomib Citrate), Niraparib Tosylate Monohydrate, Nivolumab, Nolvadex (Tamoxifen Citrate), Nplate (Romiplostim), Obinutuzumab, Odomzo (Sonidegib), OEPA, Ofatumumab, OFF, Olaparib, Olaratumab, Omacetaxine Mepesuccinate, Oncaspar (Pegaspargase), Ondansetron Hydrochloride, Onivyde (Irinotecan Hydrochloride Liposome), Ontak (Denileukin Diftitox), Opdivo (Nivolumab), OPPA, Osimertinib, Oxaliplatin, Paclitaxel, Paclitaxel Albumin-stabilized Nanoparticle Formulation, PAD, Palbociclib, Palifermin, Palonosetron Hydrochloride, Palonosetron Hydrochloride and Netupitant, Pamidronate Disodium, Panitumumab, Panobinostat, Paraplat (Carboplatin), Paraplatin (Carboplatin), Pazopanib Hydrochloride, PCV, PEB, Pegaspargase, Pegfilgrastim, Peginterferon Alfa-2b, PEG-Intron (Peginterferon Alfa-2b), Pembrolizumab, Pemetrexed Disodium, Perjeta (Pertuzumab), Pertuzumab, Platinol (Cisplatin), Platinol-AQ (Cisplatin), Plerixafor, Pomalidomide, Pomalyst (Pomalidomide), Ponatinib Hydrochloride, Portrazza (Necitumumab), Pralatrexate, Prednisone, Procarbazine Hydrochloride, Proleukin (Aldesleukin), Prolia (Denosumab), Promacta (Eltrombopag Olamine), Propranolol Hydrochloride, Provenge (Sipuleucel-T), Purinethol (Mercaptopurine), Purixan (Mercaptopurine), Radium 223 Dichloride, Raloxifene Hydrochloride, Ramucirumab, Rasburicase, R-CHOP, R-CVP, Recombinant Human Papillomavirus (HPV) Bivalent Vaccine, Recombinant Human Papillomavirus (HPV) Nonavalent Vaccine, Recombinant Human Papillomavirus (HPV) Quadrivalent Vaccine, Recombinant Interferon Alfa-2b, Regorafenib, Relistor (Methylnaltrexone Bromide), R-EPOCH, Revlimid (Lenalidomide), Rheumatrex (Methotrexate), Ribociclib, R-ICE, Rituxan (Rituximab), Rituxan Hycela (Rituximab and Hyaluronidase Human), Rituximab, Rituximab and, Hyaluronidase Human, Rolapitant Hydrochloride, Romidepsin, Romiplostim, Rubidomycin (Daunorubicin Hydrochloride), Rubraca (Rucaparib Camsylate), Rucaparib Camsylate, Ruxolitinib Phosphate, Rydapt (Midostaurin), Sclerosol Intrapleural Aerosol (Talc), Siltuximab, Sipuleucel-T, Somatuline Depot (Lanreotide Acetate), Sonidegib, Sorafenib Tosylate, Sprycel (Dasatinib), STANFORD V, Sterile Talc Powder (Talc), Steritalc (Talc), Stivarga (Regorafenib), Sunitinib Malate, Sutent (Sunitinib Malate), Sylatron (Peginterferon Alfa-2b), Sylvant (Siltuximab), Synribo (Omacetaxine Mepesuccinate), Tabloid (Thioguanine), TAC, Tafinlar (Dabrafenib), Tagrisso (Osimertinib), Talc, Talimogene Laherparepvec, Tamoxifen Citrate, Tarabine PFS (Cytarabine), Tarceva (Erlotinib Hydrochloride), Targretin (Bexarotene), Tasigna (Nilotinib), Taxol (Paclitaxel), Taxotere (Docetaxel), Tecentriq, (Atezolizumab), Temodar (Temozolomide), Temozolomide, Temsirolimus, Thalidomide, Thalomid (Thalidomide), Thioguanine, Thiotepa, Tisagenlecleucel, Tolak (Fluorouracil—Topical), Topotecan Hydrochloride, Toremifene, Torisel (Temsirolimus), Tositumomab and Iodine I 131 Tositumomab, Totect (Dexrazoxane Hydrochloride), TPF, Trabectedin, Trametinib, Trastuzumab, Treanda (Bendamustine Hydrochloride), Trifluridine and Tipiracil Hydrochloride, Trisenox (Arsenic Trioxide), Tykerb (Lapatinib Ditosylate), Unituxin (Dinutuximab), Uridine Triacetate, VAC, Vandetanib, VAMP, Varubi (Rolapitant Hydrochloride), Vectibix (Panitumumab), VeIP, Velban (Vinblastine Sulfate), Velcade (Bortezomib), Velsar (Vinblastine Sulfate), Vemurafenib, Venclexta (Venetoclax), Venetoclax, Verzenio (Abemaciclib), Viadur (Leuprolide Acetate), Vidaza (Azacitidine), Vinblastine Sulfate, Vincasar PFS (Vincristine Sulfate), Vincristine Sulfate, Vincristine Sulfate Liposome, Vinorelbine Tartrate, VIP, Vismodegib, Vistogard (Uridine Triacetate), Voraxaze (Glucarpidase), Vorinostat, Votrient (Pazopanib Hydrochloride), Vyxeos (Daunorubicin Hydrochloride and Cytarabine Liposome), Wellcovorin (Leucovorin Calcium), Xalkori (Crizotinib), Xeloda (Capecitabine), XELIRI, XELOX, Xgeva (Denosumab), Xofigo (Radium 223 Dichloride), Xtandi (Enzalutamide), Yervoy (Ipilimumab), Yondelis (Trabectedin), Zaltrap (Ziv-Aflibercept), Zarxio (Filgrastim), Zejula (Niraparib Tosylate Monohydrate), Zelboraf (Vemurafenib), Zevalin (Ibritumomab Tiuxetan), Zinecard (Dexrazoxane Hydrochloride), Ziv-Aflibercept, Zofran (Ondansetron Hydrochloride), Zoladex (Goserelin Acetate), Zoledronic Acid, Zolinza (Vorinostat), Zometa (Zoledronic Acid), Zydelig (Idelalisib), Zykadia (Ceritinib), and/or Zytiga (Abiraterone Acetate). Also contemplated herein are chemotherapeutics that are PD1/PDL1 blockade inhibitors (such as, for example, lambrolizumab, nivolumab, pembrolizumab, pidilizumab, BMS-936559, Atezolizumab, Durvalumab, or Avelumab). It is also intended herein that the disclosed uses of the disclosed compositions and/or an engineered NK cell population for inhibiting, reducing, and/or preventing cancer metastasis and/or recurrence can comprise use in combination the use of any anti-cancer agent known in the art including, but not limited to those agents listed above.

Dosing frequency for the therapeutic agent disclosed herein, includes, but is not limited to, at least once every 12 months, once every 11 months, once every 10 months, once every 9 months, once every 8 months, once every 7 months, once every 6 months, once every 5 months, once every 4 months, once every 3 months, once every two months, once every month; or at least once every three weeks, once every two weeks, once a week, twice a week, three times a week, four times a week, five times a week, six times a week, or daily. In some embodiments, the interval between each administration is less than about 4 months, less than about 3 months, less than about 2 months, less than about a month, less than about 3 weeks, less than about 2 weeks, or less than less than about a week, such as less than about any of 6, 5, 4, 3, 2, or 1 day. In some embodiments, the dosing frequency for the nanoparticle composition includes, but is not limited to, at least once a day, twice a day, or three times a day. In some embodiments, the interval between each administration is less than about 48 hours, 36 hours, 24 hours, 22 hours, 20 hours, 18 hours, 16 hours, 14 hours, 12 hours, 10 hours, 9 hours, 8 hours, or 7 hours. In some embodiments, the interval between each administration is less than about 24 hours, 22 hours, 20 hours, 18 hours, 16 hours, 14 hours, 12 hours, 10 hours, 9 hours, 8 hours, 7 hours, or 6 hours. In some embodiments, the interval between each administration is constant. For example, the administration can be carried out daily, every two days, every three days, every four days, every five days, or weekly. Administration can also be continuous and adjusted to maintaining a level of the compound within any desired and specified range.

### Methods of Identifying INTmers

Great progress has been made on designing ASOs with greater bioavailability and stability. However, despite these advances, difficulties remain to obtain ASOs with high specific activity against protein coding genes. This is mainly due to the fact that there are no rules as to what region(s) of the transcript to target the ASOs against and unlike siRNAs, most ASOs against exons, introns or 3′UTR don't display robust silencing activity. Therefore, herein provided is a key advance in anti-sense oligonucleotides technology through the ease of designing and screening INTmers complementary to transcriptional start site sequences to arrive at potent molecules for silencing of the gene of interest.

Accordingly, in some aspects, disclosed herein is method for identifying INTmers which are capable of downregulating gene expression of a gene of interest, the method comprising: a) sequencing at least the first 150 nucleotides of a region at a 5′ end of an RNA transcript of the gene of interest, or sequencing the gene of interest itself; b) creating a library of INTmers which are at least 80% complementary to region within the first 150 nucleotides of the RNA transcript of the gene of interest sequenced in step a), wherein said INTmers comprises a nucleic acid molecule comprising 16-20 nucleotides, and further wherein said nucleic acid molecule comprises a central core of DNA flanked by RNA on each side; c) exposing the INTmer library created in step b) to mammalian primary cells or cell lines expressing the nascent RNA transcript complementary to the INTmer sequencies; and d) determining which INTmers are capable of downregulation of gene expression of the gene of interest.

In some embodiments, the INTmer is 60% or more (e.g., 60% or more, 70% or more, 80% or more, 90% or more, 95% or more, 99% or more, or 100%) complementary to the RNA transcript.

In some embodiments, the INTmer hybridizes to the nascent mRNA transcript of the gene of interest within the first 350 base pairs (including, for example, the first 300 base pairs, first 250 base pairs, the first 200 base pairs, the first 150 base pairs, the first 100 base pairs, the first 50 base pairs, the first 45 base pairs, the first 40 base pairs, the first 35 base pairs, the first 30 base pairs, the first 25 base pairs, or the first 20 base pairs, the first 10 base pairs) of a 5′ end of the nascent mRNA transcript.

In some embodiments, the INTmer hybridizes to the nascent mRNA transcript of the gene of interest within nucleotide positions 1 to 300, nucleotide positions 1 to 100, nucleotide positions 1 to 50, nucleotide positions 5 to 250, nucleotide positions 5 to 200, nucleotide positions 5 to 150, nucleotide positions 5 to 100, nucleotide positions 10 to 150, nucleotide positions 10 to 100, nucleotide positions 10 to 80, nucleotide positions 10 to 50, nucleotide positions 20 to 200, nucleotide positions nucleotide positions 20 to 150, nucleotide positions 20 to 100, nucleotide positions 20 to 50, nucleotide positions 30 to 300, nucleotide positions 30 to 200, nucleotide positions 30 to 150, nucleotide positions 30 to 100, nucleotide positions 50 to 75, nucleotide positions 50 to 100, nucleotide positions 50 to 150, nucleotide positions 50 to 200, nucleotide positions 50 to 250, nucleotide positions 50 to 300, nucleotide positions 100 to 350 nucleotide, or positions 100 to 200 from a 5′ end of the nascent mRNA transcript.

The INTmer can be 5 to 100 nucleotides long (including, for example, 10 nucleotides long, 11 nucleotides long, 12 nucleotides long, 13 nucleotides long, 14 nucleotides long, 15 nucleotides long, 16 nucleotides long, 17 nucleotides long, 18 nucleotides long, 19 nucleotides long, 20 nucleotides long, 21 nucleotides long, 22 nucleotides long, 23 nucleotides long, 24 nucleotides long, 25 nucleotides long, 26 nucleotides long, 27 nucleotides long, 28 nucleotides long, 29 nucleotides long, 30 nucleotides long, 40 nucleotides long, 60 nucleotides long, 80 nucleotides long, or 100 nucleotides long). In some embodiments, the INTmer comprises a nucleic acid molecule comprising 14-22 nucleotides.

The INTmer comprises central core of DNA flanked by RNA on each side. In some examples, the central core is 5, 6, 7, 8, 9, 10, 11, 12, 13, 14, 15, 16, 17, 18, 19, or 20 nucleotides in length. In some embodiments, the central core comprises at least one modified nucleotide. In some examples, the modified nucleotide comprises phosphorothioate and/or methylated at C5 position of cytosine. In some embodiments, each flanking RNA is 2, 3, 4, 5, 6, 7, 8, 9, or 10 nucleotides in length on either side of the central core. In some embodiments, the RNA of the INTmer is modified (for example, the modified RNA is a 2′-O-methoxy (MOE)-modified RNA and/or a locked nucleic acid (LNA).

As herein contemplated, the INTmer can hybridized within the first 350 base pairs of a 5′ end of the nascent mRNA transcript, including 200 base pairs where any single nucleotide can be marked by 7-methylguanylate (a CAP structure) followed by, for example, 150 nucleotides of nascent mRNA.

In some embodiments, the method is carried out in cells in vitro. In some embodiments, the cells are cancer cells. In some embodiments, in step d), determining which INTmers are capable of downregulation of gene expression is carried out by measuring RNA levels of transcripts of the gene of interest.

In some embodiments, RT-qPCR is used to measure RNA levels. In some embodiments, protein levels of a protein corresponding to a gene of interest are measured (e.g., measured using Western Blotting).

In some embodiments, cellular viability is measured.

In some embodiments, the INTmer is designed against an RNA polymerase II pause-release domain. In some embodiments, the pause-release domain corresponds to about nucleotides 40 to 350 of the nascent RNA transcript. In some embodiments, the pause-release domain corresponds to about nucleotides 40 to 150 of the nascent RNA transcript.

In some embodiments, the INTmer is designed against an RNA polymerase promoter-escape region. In some examples, the promoter-escape region corresponds to about nucleotides 20 to 80 of the nascent RNA transcript.

### EXAMPLES

### Example 1. Introduction

Recent studies have indicated that RNA polymerase II (RNAPII) in human cells encounters two key barriers or checkpoints to initiation of transcription. The first is known as “Promoter-Escape” where RNAPII disengages from the initiation factors when the nascent RNA reaches about to 20 nucleotides from transcriptional start site. This step requires conformational changes by transcription factor TFIIB and perhaps structural changes in R-loop formation where the nascent transcript makes complementary interactions with non-template DNA strand to form RNA-DNA hybrids. Following promoter escape, RNAPII stalls around nucleotides 40 to 100 close to +1 nucleosome in a phenomenon described as RNAPII “Pause-Release”. To overcome the barrier that RNAPII encounters at this checkpoint requires participation of a set of factors known as pause-release factors including NELF, SPT5/SPT4 (DSIF), and the multi-subunit RNAPII-associated Integrator complex. Importantly, Integrator complex through the use of its catalytic endonuclease activity cleaves the stalled nonproductive transcript (where RNAPII is paused) to prematurely terminate transcription allowing for new rounds of transcription initiation. Since transcriptional initiation and their newly initiated transcripts (nucleotides 20 to 100) are highly regulated, subject to R-loop formation and endonucleolytic cleavage by Integrator complex, it provides a key vulnerability for gene silencing.

To achieve gene silencing at initiation, the activity of improved antisense oligonucleotides (ASOs) was tested for in vivo applications known as GAPmers (18 nucleotides), consisting of a central core of modified (phosphorothioate) DNA (10 mers) flanked by nuclease-resistant 2′-O-methoxy (MOE)-modified RNAs (4-10-4), targeted to RNA sequences surrounding the start site of transcription as defined by CAGE and extending beyond paused RNAPII. These were termed anti-initiation ASOs, Initiation of Transcription GAPmers or (INTmers). INTmers are complementary to start site sequences forming an RNA-DNA hybrid which is recognized by RNase H1 and cleaved down-stream of start sites in nascent RNA. Indeed, this sustained Anti-Initiator-induced RNase H1 cleavage of nascent transcripts is reminiscent of Integrator complex cleavage of nonproductive initiated transcripts. Importantly, cleavage of the mRNAs close to transcription start sites has multiple advantages including silencing a disease-specific or disease-prevalent longer transcripts initiated from an upstream transcriptional start sites. This can also deem relevant in case of silencing of a longer transcript where a longer isoform is expressed predominantly in a tissue-specific manner. Additionally, cleaving the transcripts close to transcription start sites allows for removal of the 5′-methyl-CAP and further destabilization of mRNAs. Proof of principle evidence is presented herein to show that targeting EGFR and BRAF using INTmers are highly effective strategy to silence gene expression. Specifically in case of BRAF where a longer transcript is expressed in melanoma cells, the key unique aspect of targeting transcriptional initiation to silence disease-specific transcription is demonstrated.

While gene silencing in tissue culture cells could be performed with RNA interference (RNAi), the use of RNAi for in vivo therapeutic applications have proven difficult. There has been great effort by pharmaceutical industry including to develop RNase H1-mediated ASO technology to silence pathogenic gene expression in vivo. While, there has been a number of success stories for blocking ASOs (to interfere with RNA binding proteins) in order to alter splicing, there have been major barriers in using RNase H1-mediated GAPmers. The key issue has been the lack of any insight as where to target a messenger RNA for RNase H1-directed cleavage. Overall, there are no rules as to what sequences to target and most approaches involve a laborious random screening of a few thousand ASOs directed to exons, introns or 3′-untranslated region in hope of finding an effective ASO. This critical gap in knowledge has been addressed by using the ASOs to explore the key vulnerabilities in transcriptional initiation. INTmers consist of 18 nucleotide GAPmers complementary to a genomic region that is on average 100 nucleotides in length intended to target the key bottlenecks in transcriptional initiation. Consequently, by assessing the activity of approximately 20 INTmers, the study herein shows a number of potent GAPmers capable of silencing disease-specific gene expression at low nanomolar levels in vivo. These molecules can be used as potent inhibitory drugs against the targeted pathogenic transcripts in any disease models they would be deployed. Herein the study assesses the utility of this approach and the effectiveness of INTmers in a number of human cancers where activating mutations in MAPK pathway drives the pathogenesis.

### Example 2. A New Direction: Initiation of Transcription Interference (INTi) Offers a New Genome-Wide Platform for Pathogenic Gene Silencing

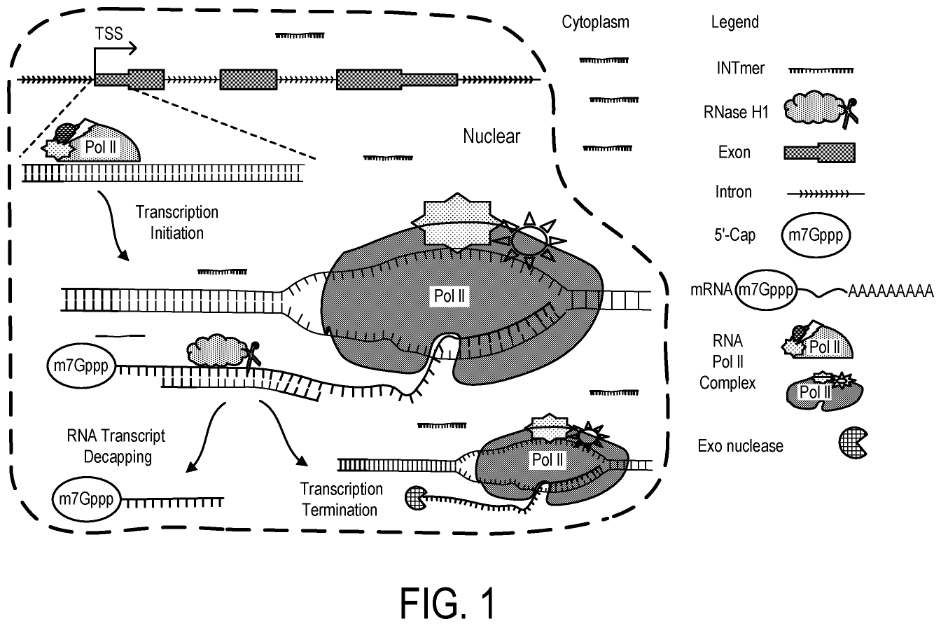

Shown herein is a new platform for gene silencing based on anti-sense oligonucleotides (ASOs) targeting of RNase H1 to transcriptional start sites of human genes (FIG. 1). This technology termed Initiation of Transcription Interference (INTi), is a highly effective modality for silencing pathogenic gene expression. Derivatized ASOs suitable have been developed for in vivo preclinical experiments targeted to the transcriptional start sites, termed INTmers. This INTi opens the possibility of silencing previously undruggable targets such as NRAS, specifically. It is important to consider that cancer cells with activated oncogenes are potently addicted to the levels of the corresponding oncogene and therefore reduction of the oncogene dosage has profound deleterious effects on cancer cells. Since activating mutations of some genes are key drivers of large number of human cancers including pancreatic, colon, leukemia, skin, among others, application of INTi has an unparalleled impact in cancer therapy. Additionally, INTi mode of gene silencing can be adopted to treating any human disease suffering from pathogenic gene expression including neurodegenerative or inflammatory diseases.

Shown herein is therapeutic silencing of key MAPK oncogenes (EGFR, BRAF, NRAS, and HRAS) in preclinical models of pancreatic, lung, colon and skin cancers. This encompasses over 70% of human solid tumors. The study additionally investigates the application of INTi to therapeutic treatment of human cancers with either intrinsic or acquired resistance to targeted therapy against inhibitors of MAPK pathway. These efforts are expanded to experiments assessing a combination therapy using INTi and treatments such as PARP inhibitors or apoptotic inhibitors of BCL2 or MCL1 as appropriate.

### Example 3. Develop Atlases of Transcription Start Sites and Paused RNAPII Profiles in Cancers Driven by Activating Mutations in Oncogenes

Analysis of cancer genome atlas (TCGA) indicates that nearly 90% of some cancers regardless of their tissue of origin (including pancreas, lung, colorectal cancers) result from an activating substitution of a single amino acid to many different amino acids. BRAF is an example where a large number of melanomas and other cancer types arise from a single missense mutation in Valine at position 600 (V⁶⁰⁰). American type culture collection (ATCC) has compiled a large collection of cancer cell lines that contain defined mutations in cancer genes including activating mutations of BRAF V⁶⁰⁰. The present study testes a large number of pancreatic, colon, lung and skin cancer cell lines with activating mutations of key oncogenes in the mitogen-activated protein kinase (MAPK) pathway.

This study first defines the landscape of transcription start sites (TSS) for activated oncogenes in these mutant-driven cell lines and human tumors containing activated oncogenes. Activated oncogenes in MAPK pathway (EGFR, BRAF, NRAS, HRAS) are focused on since they are the drivers of a large number of human cancers. The oncogenes in lung, pancreatic, colon and skin cancers are tested using cell lines and tumor samples. Particularly, study explores the INTi technology in cancers with intrinsic or acquired resistance to targeted therapy. Late stage skin cancer as well as pancreatic and lung cancers are among the deadliest in the United States and are predominantly caused by an over-active MAPK pathway. Therefore, finding an effective therapeutic modality for these cancers is a giant leap forward in cancer therapy.

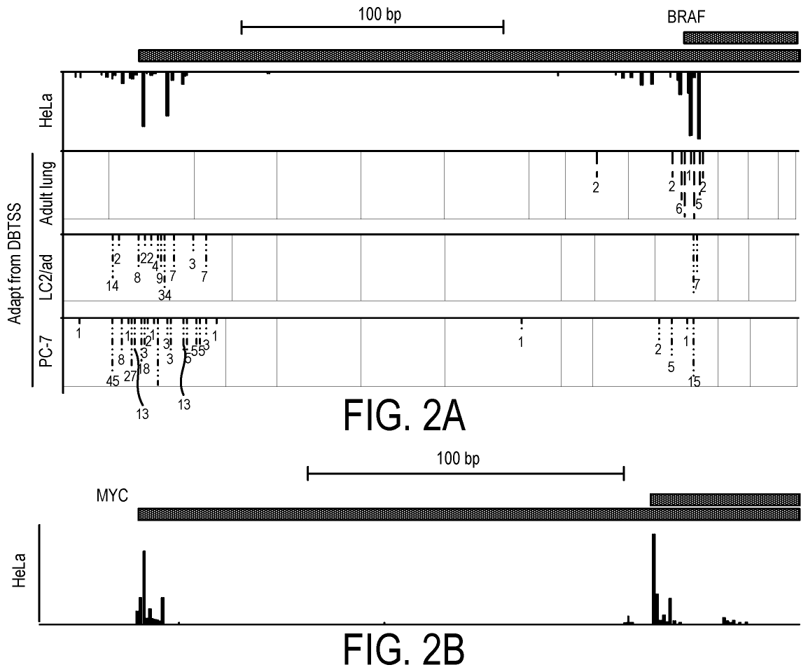

Next performed is cap analysis gene expression (CAGE) and the chromatin marks H3K27ac and H3K4 trimethyl using genome-wide technologies. This allows the determination of precise transcription start sites of activated oncogene for each cancer cell line and patient-derived tumor sample as well as confirmation of its nucleosome accessibility by displaying activating chromatin marks at +1 and −1 nucleosomes flanking the transcription start sites. Many of critical human oncogenes including BRAF and Myc have multiple transcriptional start sites that depending on the degree of promoter usage are differentially utilized in different tissues and during pathogenesis. FIG. 2 depicts the CAGE analyses for MYC in HeLa cells and BRAF in normal lung epithelial and lung cancers. Interestingly, both genes show two distinct transcription start sites. It is noteworthy that in normal lung epithelial cells BRAF is only expressed from the shorter transcript nearly 200 nucleotides down-stream of longer isoforms in lung cancer cells.

Example 4. Using these Atlases, INTmers (GAPmer) Complementary to Regions of Transcription Initiation Checkpoints (Promoter-Escape and Paused RNAPII) of Activated Oncogenes are Screened to Arrive at INTmers that Silence Cancer-Specific Isoforms

Once the TSS of activated oncogenes is determined in different cancer cell lines and human tumor samples, INTmers (GAPmers to initiation sites) are developed against individual TSS of each oncogene in its relevant cancer cell lines and tumors. This study screens 20 INTmers directed against sequences corresponding to promoter-escape (nucleotides 20 to 40) and RNAPII pause-release (nucleotides 40 to 100). The INTmers are screened using three concentrations (100, 200 and 400 nM) using multiple readouts. The steady state RNA levels are measured using RT-qPCR, protein levels are measured using western blotting and cellular viability is measured at each concentration. Once the study determines the INTmer that display high specific activity in silencing the activated oncogene, the silencing activity can be optimized by developing INTmers with modified chemistry such as locked nucleic acids (LNA) which are more costly for the initial screening. The silencing activity of INTmers is compared in similar cancers where the mutant oncogene is expressed in wild type form.

### Development of a New Gene-Silencing Platform.

Targeting transcription initiation checkpoints to regulate locus-specific gene expression. It is important to consider the current limitations of RNase H1-mediated ASO silencing of gene expression. The last twenty years have witnessed great progress in designing ASOs with greater bioavailability and stability due to key changes in the chemistry of DNA backbone and modified flanking bases allowing increased pairing activity. However, despite these advances, most experimental work for gene silencing in the laboratory setting use RNA interference technology since it is proven difficult to obtain ASOs with high specific activity against protein coding genes. This is mainly due to the fact that there are no rules as to what region(s) of the transcript to target the ASOs against and unlike siRNAs, most ASOs against exons, introns or 3‘UTR don’t display robust silencing activity. The targeting problem is exasperated since most primary transcripts are thousands of base pairs compelling most pharmaceutical companies to screen thousands of ASOs, which is beyond the capability of individual laboratories. Therefore, this study provides a key advance in anti-sense oligonucleotides technology through the ease of designing and screening INTmers complementary to transcriptional start site sequences to arrive at potent molecules for silencing of the gene of interest.

INTmers silence oncogenic EGFR: To show the effectiveness of INTmers in silencing of activated oncogenes, a number of INTmers were designed to the start site of EGFR gene, a growth factor receptor whose activating mutations are drivers of large number of cancers including lung adenocarcinomas and glioblastomas. The study used 18 nucleotide GAPmers with a 10 nucleotide central modified DNA flanked on each side by 2MOE modified 4 RNA complementary bases (4-10-4). The DNA sequences were intended to pair with complementary 5′-end sequences of EGFR transcript and act as a substrate for RNase H1 cleavage activity.

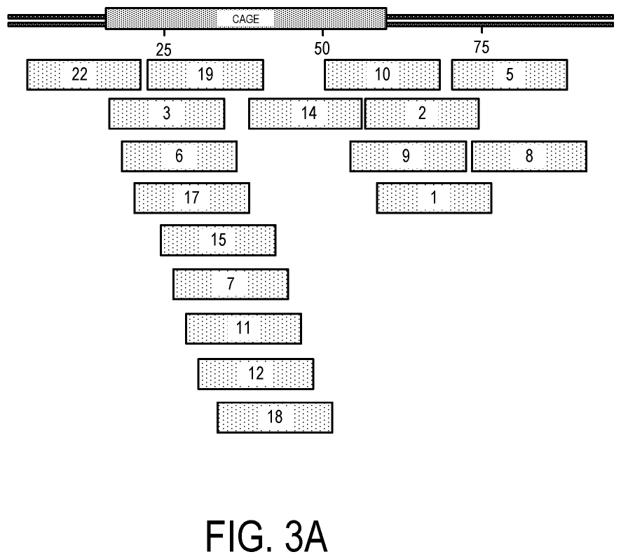

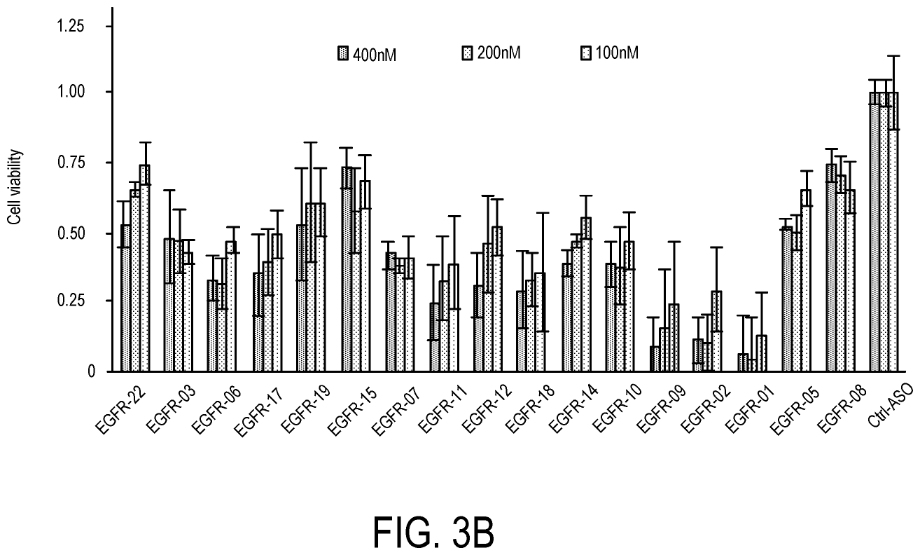

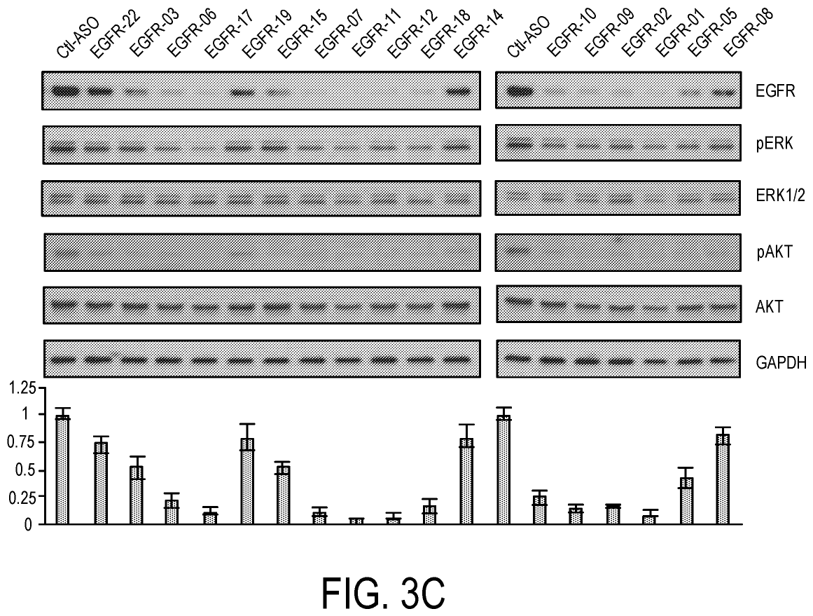

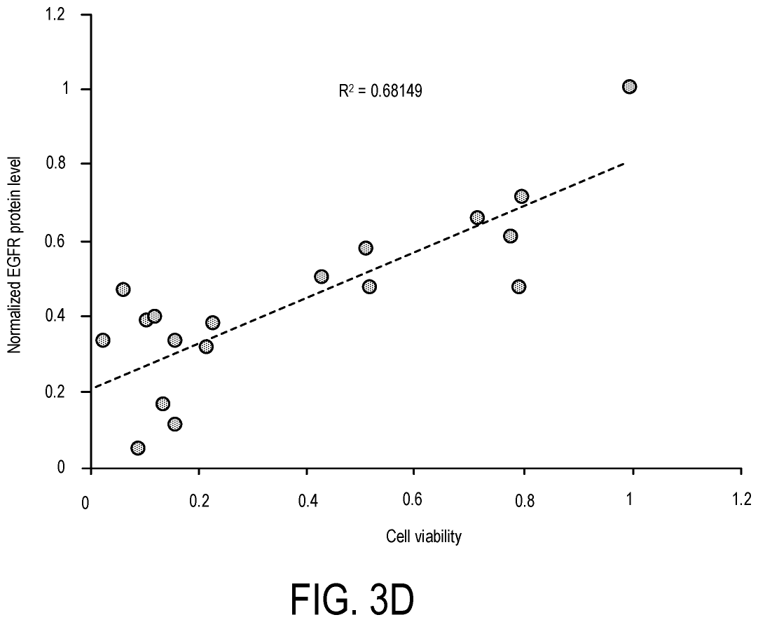

Defining the transcriptional start sit of EGFR: The start sites of human genes are not defined by a single nucleotide and often represented by a cluster of nucleotides that are different among genes. Analysis of CAGE results indicates that transcription start sites can be approximately 50 nucleotides wide. Importantly, different genes display different patterns of start sites that display differences in a tissue-dependent manner depending on the promoter architecture. Human transcription start sites are governed based on basal general transcription factor binding to core promoter elements and are usually some 30 nucleotides downstream of TBP binding. Most genomic start sites are represented by predominant nucleotides at annotated TSS with minor CAGE signals flanking the annotated start site. 17 INTmers spanning the EGFR CAGE signal were designed based on the results obtained in HeLa cells which is in agreement with the annotated start site of EGFR (FIG. 3A). The INTmers were developed to be optimal for complementarity and RNase H1 cleavage activity covering an area of approximately 100 nucleotides. NCI-H1975 was initially used, a lung cancer cell line harboring double mutations in EGFR (L858R/T790M) to screen for cell viability using three concentrations (100, 200 and 400 nM) of INTmers. While all INTmers displayed some growth inhibitory activity, a cluster of INTmers designated by EGFR-01, -02 and -09 showed enhanced growth inhibitory activity even at 100 nM (FIG. 3B). This cluster maps to about 25 to 45 nucleotides from the start of transcription consistent with nascent RNA engagement in promoter escape. Indeed, western blot analysis of H1975 cells for EGFR levels following treatment with 150 nM of INTmers confirmed the potent decrease in EGFR levels concomitant with growth inhibition (FIGS. 3C-3D). These results reveal that the INTmers directed to transcriptional start sites are a potent means to inhibit gene expression.

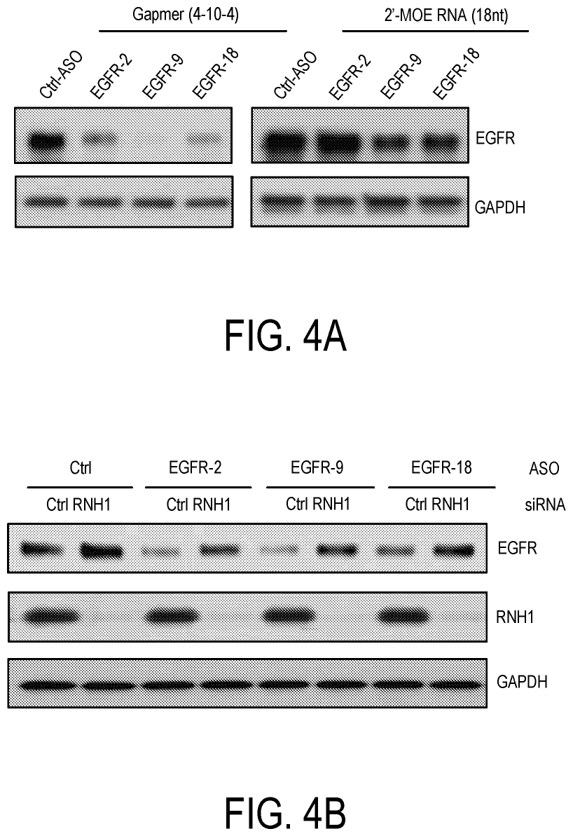

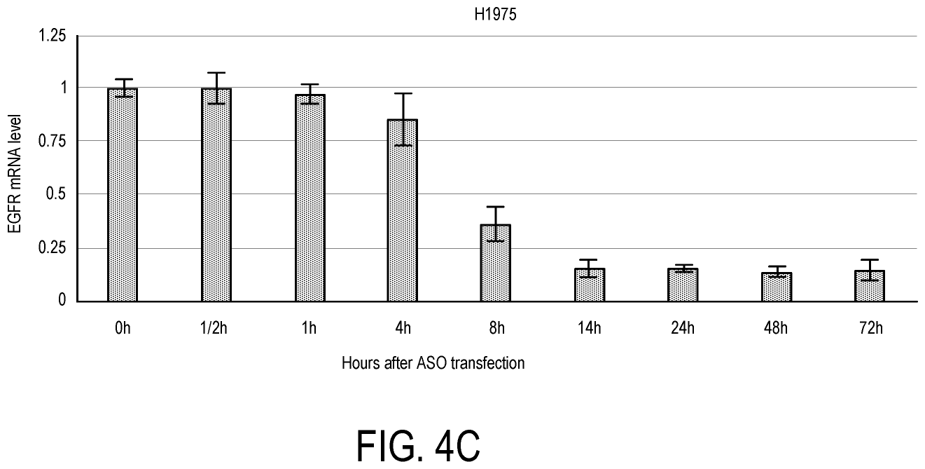

Mechanism of action of INTmers: To show that the INTmers specifically inhibited EGFR expression, a set of ASOs were devised with identical sequences to effective INTmers with the exception of having a composition of uniform 2′-MOE-RNA modified bases which are not recognized by RNase H1. Unlike INTmers, the uniform 2′-MOE oligos had little effect on EGFR expression (FIG. 4A). Additionally, knockdown of RNase H1 abrogated the inhibitory activity of INTmers against EGFR expression demonstrating that the mechanism of silencing is through RNase H1 cleavage activity (FIG. 4B). The data show that INTmers silenced expression of their target gene within a few hours following treatment of cells where decreased level of EGFR was evident as early as 8 hours following treatment and persisted for 72 hours following a single treatment of INTmer (FIG. 4C).

Do INTmers function by prematurely terminating the nascent transcript? Since transcriptional initiation is subject to premature termination as a critical regulatory mechanism and transcriptional start sites are prominent genomic locations decorated with R-loops, INTmer RNase H1-mediated targeted silencing of gene expression can impact nascent transcripts in early phases of transcriptional initiation. Real-time PCR primers to primary and mature transcripts are designed and the effects of INTmers on the levels of nascent as well as steady state transcript levels are examine. It is important to note that a pronounce decrease in mRNA steady state levels was observed within 8 hours following treatment with the INTmers (FIG. 4C). These experiments can be extended by analyzing the changes in mRNA CAP binding. Transcripts are immunoprecipitated using antibodies against the 5′ CAP structure and the level of primary and mature transcripts are measured using RT-qPCR. This can be due to a large loss in the 5′-CAP structure since INTmer-mediated targeted cleavage of the transcript by RNase H1 is to de-CAP the transcripts. To further assess whether transcription is inhibited during pioneering round of transcriptional initiation, chromatin immunoprecipitation is performed followed by real-time PCR (ChIP-qPCR) for RNAPII at transcription start site of EGFR, BRAF, NRAS, HRAS genes following treatment of cells with INTmers or control ASOs. If INTmers induce premature termination of nascent transcripts, a large diminution in RNAPII occupancy can be observed at the start site of INTmer treated oncogenes.

Does RNase H1 require additional factors for recruitment to its target sites or activity in vivo? To gain a better understanding of the mechanism by which RNase H1 mediate the INTmers-directed silencing of transcripts at start sites, ChIP-seq and ChIP-mass spectrometry are performed in the presence and absence of INTmers. Recent studies show that stably transfected cells with a catalytic mutant RNase H1 provide an excellent reagent for analyses of RNase H1 genome-wide occupancy. Indeed, these studies show an enrichment of RNase H1 at start site of protein coding genes confirming previous work indicating a high enrichment of R loops at transcriptional start sites. lung cancer and melanoma cell lines stably expressing HA-tagged mutant RNase H1 catalytic mutant (D210N) are developed. These cell lines are used to perform ChIP followed by sequencing (ChIP-seq) to determine the localization of RNase H1 before and after treatment of cells with INTmers. Occupancy of RNase H1 is found at start sites of many protein-coding genes. Additionally, treatment of cancer cell lines with INTmers directs a robust increased recruitment of RNase H1 to specific loci targeted by INTmers.

These studies determine whether RNase H1 recruited to the transcriptional start sites require other factors for occupancy or activity. The studies use the HA-RNase H1 stable cell lines as well as antibodies against endogenous RNase H1 to perform ChIP followed by protein identification by mass spectrometry in the absence and presence of INTmers. Untagged cell lines and IgG are used as negative control for these experiments. These studies determine whether there are other key proteins that may be associating with RNase H1 and mediating its recruitment. The critical components found in the analyses are depleted using RNAi and their contribution to RNase H1 recruitment and gene silencing mediated through INTmers is assessed. Both nuclear and cytoplasmic tagged RNase H1 from stably expressing cells as well as endogenous RNase H1 are purified and characterized using antibodies against the protein. These purifications are subjected to mass spectrometry to determine additional RNase H1 interacting proteins in the cytoplasm or the nucleus. Specific interactors of RNase H1 are assessed to determine their function in INTmer-mediated silencing of gene expression.

Defining the therapeutic window of INTmers: A key question in using INTmers for therapeutic use against a cancer-causing activated oncogene is whether silencing of the oncogenic gene expression result in preferential cellular arrest or death of the cancer cell sparing the normal cells. To address this important question, the responsiveness of lung cancer cells with activating mutations of EGFR (H1650, H1975) is compared with lung cancers with other oncogenic aberrations (H1437, H460) and normal lung epithelial cells (BEAS-2B). The IC50 for cell viability of these 5 cell lines is measured using two distinct INTmers. The study finds a large therapeutic window for the INTmers in cells with activating mutations of the oncogene since cancer cells with activated oncogene display an addiction for sustained and enhanced activity of the mutated oncogene. Consequently, cancers with pathogenic oncogenes are highly sensitive to inhibition of the cancer driving genes. Indeed, there is a large therapeutic window between cancer cells and normal cells, which in most cases either don't express the oncogene or the expression is context dependent predominantly in response to developmental or extracellular stimuli.

Ultimate goal of cancer therapy: Cancer-specific gene silencing: Cancer-specific silencing of BRAF in melanoma: Activating mutations in BRAF is responsible for nearly 60% of melanomas as well as a large numbers of colorectal cancers, lung adenocarcinomas, multiple myelomas and nearly 90% of hairy cell leukemia. While there has been substantial progress in development of specific inhibitors to mutant BRAF, rapid emergence of resistance has hampered the therapeutic potential of BRAF inhibitors. Emergence of drug resistance against inhibitors of EGFR, BRAF and other host of protein kinases through secondary mutations is a prevailing problem in cancer therapy.

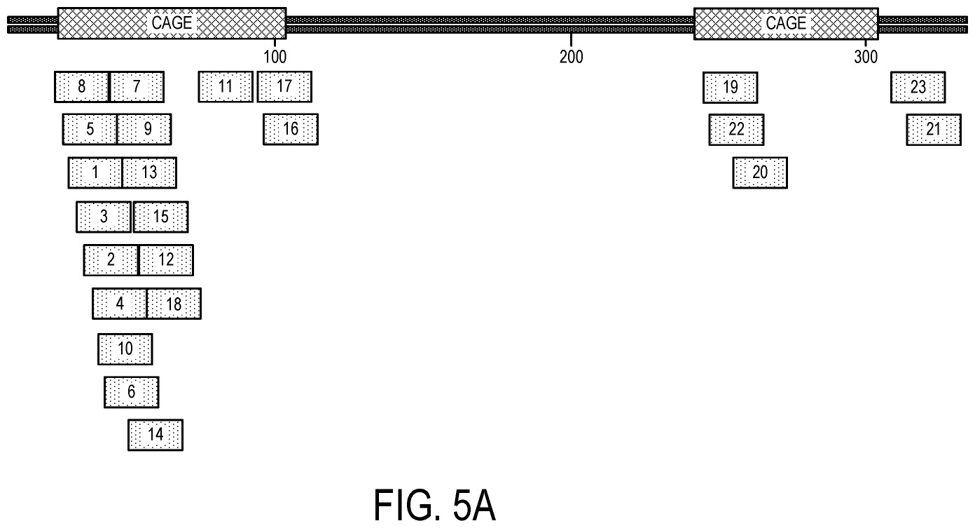

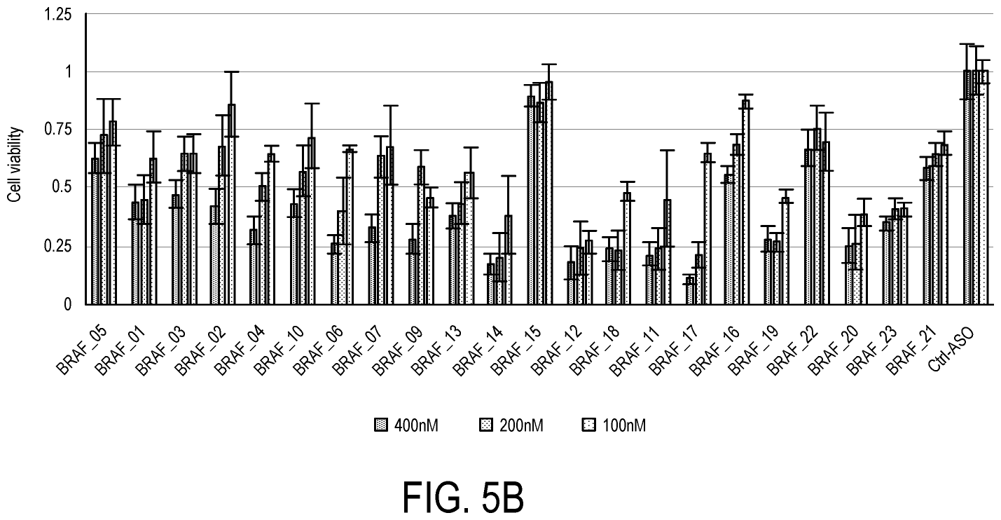

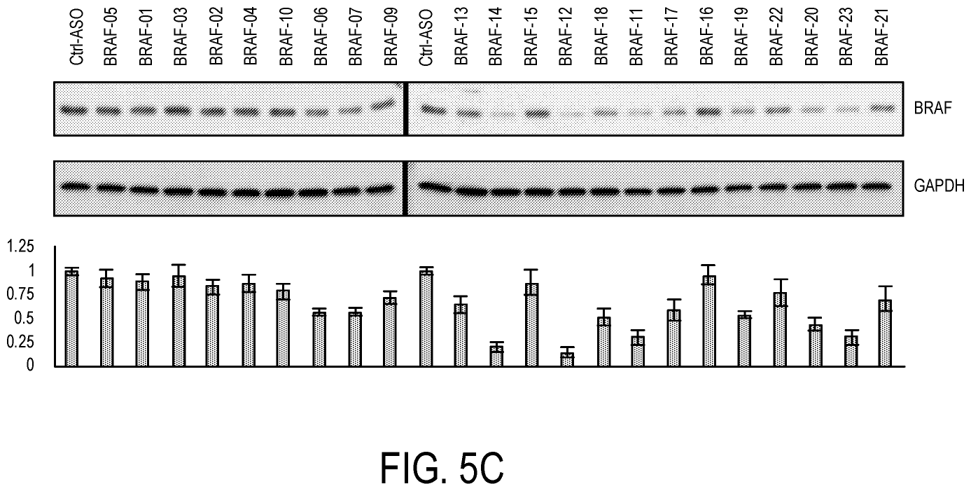

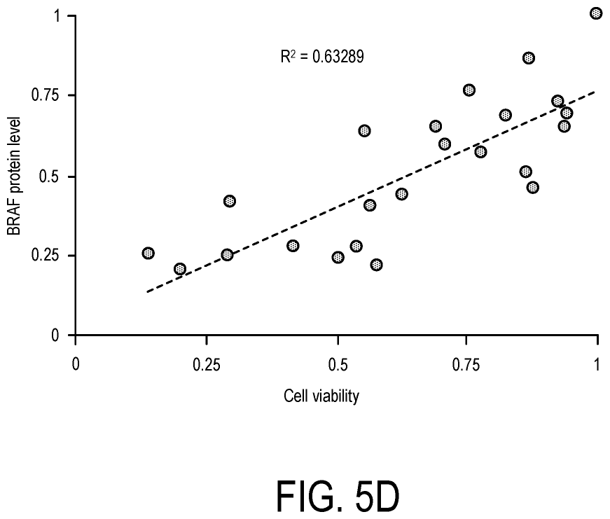

Silencing of protein kinase expression leading to the loss of protein synthesis can be an effective way to address this mode of escape from an activated oncogene. Additionally, the ability to silence cancer-specific or cancer-prevalent expression of an oncogene is an ultimate goal of any cancer therapeutics. BRAF contains two distinct transcriptional start sites about 250 nucleotides apart from each other (FIG. 2A). Transcription initiation from these start sites also result in presence of two regions where RNAPII can be found in the paused state downstream from each start site. Analyses of publicly available CAGE data indicate that while in melanoma the upstream start site is primarily used, normal melanocytes utilize the second downstream start site. Therefore, in contrast to BRAF in normal skin cells, the BRAF transcript in melanoma and most cancer types is about 150 to 200 nucleotides longer in its 5′-end. A set of INTmers are developed against BRAF's two start sites as well as its two paused RNAPII sites and performed a cell viability assay at three different concentrations of INTmers in A375 cells, a BRAF mutant melanoma cell line (FIGS. 5A-5B).

These analyses of cell viability indicates a predominant growth suppression with a number of INTmers, prominent among them BRAF-12 and BRAF-14 (FIG. 5B). Importantly, western blot analyses for protein levels correlated with growth suppression showing largest decrease in BRAF protein levels for BRAF-12 and BRAF-14 (FIG. 5C-D). These experiments highlight the effectiveness of INTmers in silencing BRAF expression leading to suppression of growth.

Silencing of the cancer-prevalent longer isoform of BRAF in melanoma: Experiments in FIG. 5 indicate two sets of INTmers directed to longer cancer-prevalent (BRAF-12 and 14) and the shorter isoform (BRAF-20 and 23) more abundant in normal cells. These sets of isoforms are targeted by INTmers to assess whether melanoma cells display a differential sensitivity to BRAF12/14 vs that of BRAF20/23. Since the longer isoform is predominantly expressed in melanoma cells, a greater inhibition of cellular growth by BRAF12/14 is found in cancer cells. Indeed, normal melanocytes should not display any sensitivity to BRAF12/14 since they don't express the longer isoform.

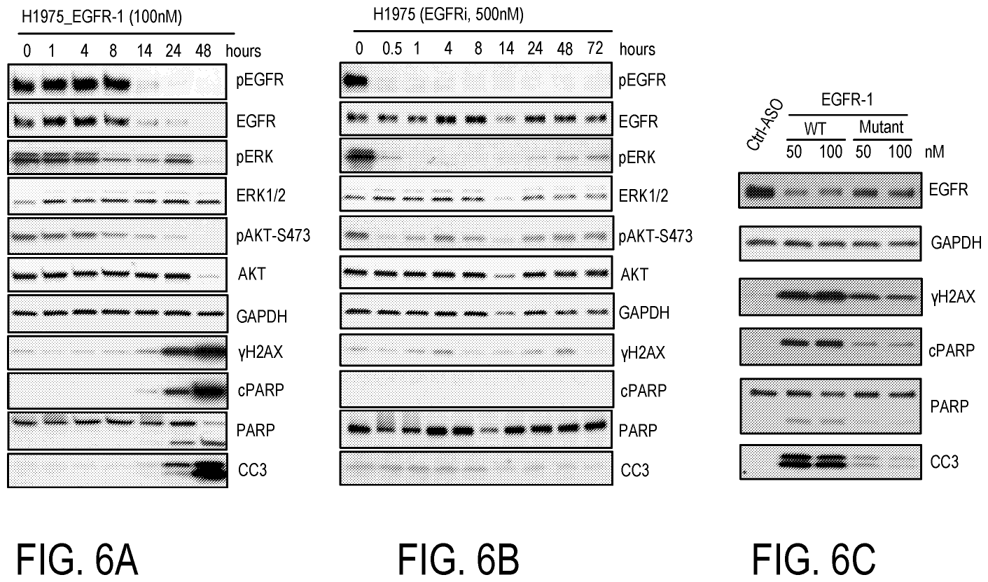

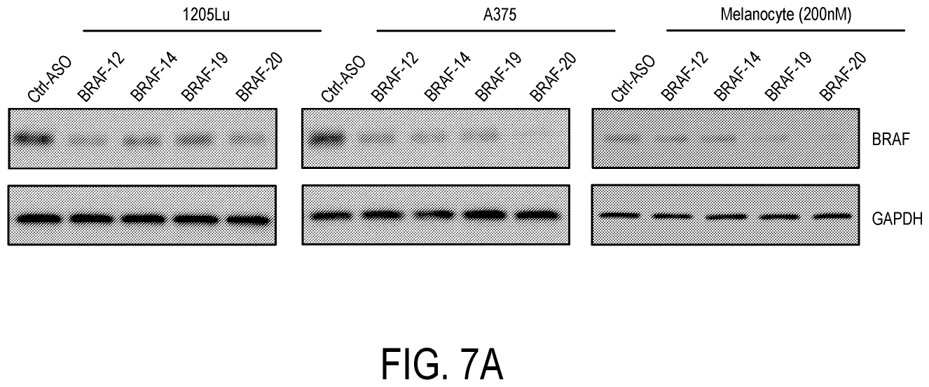

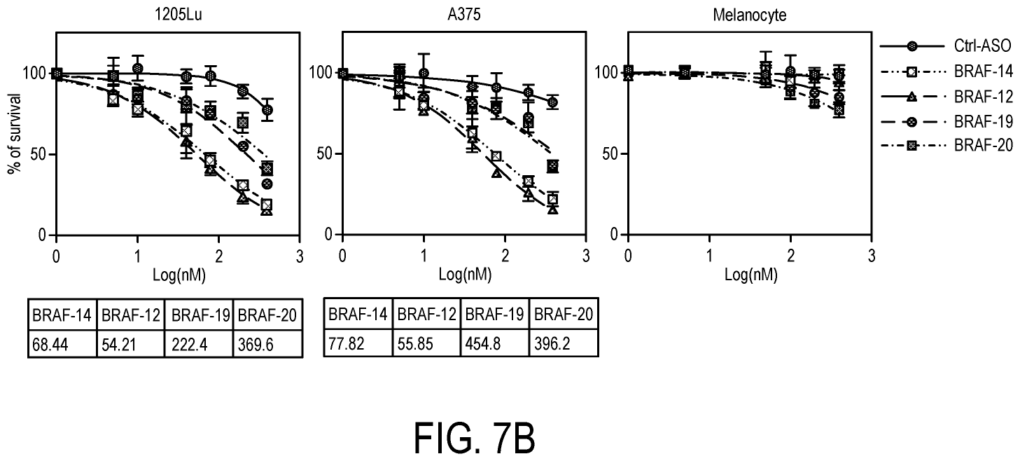

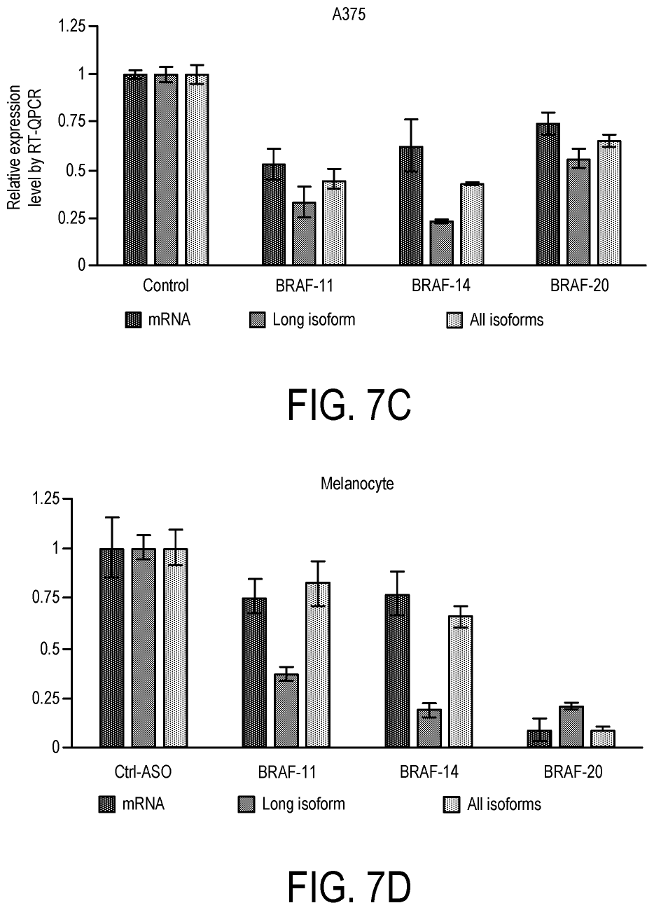

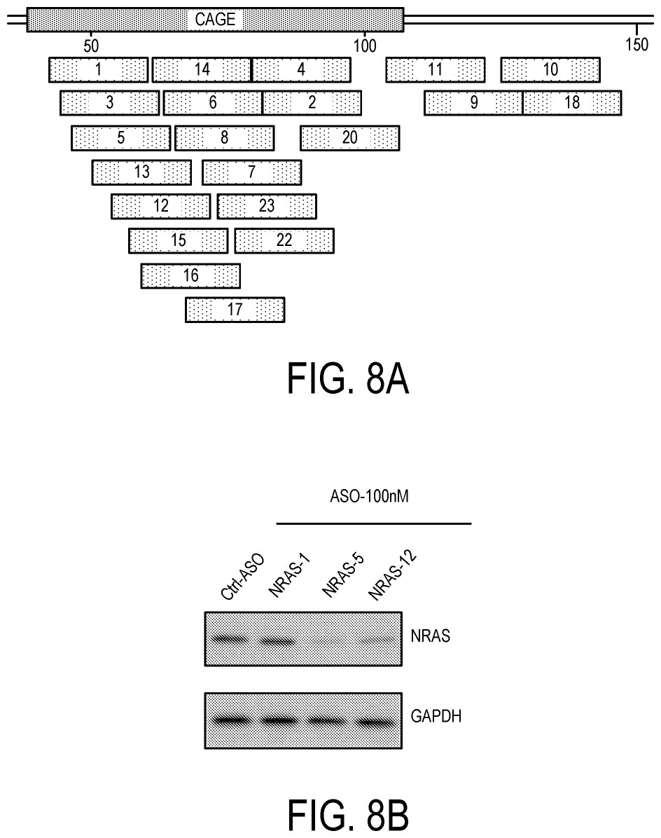

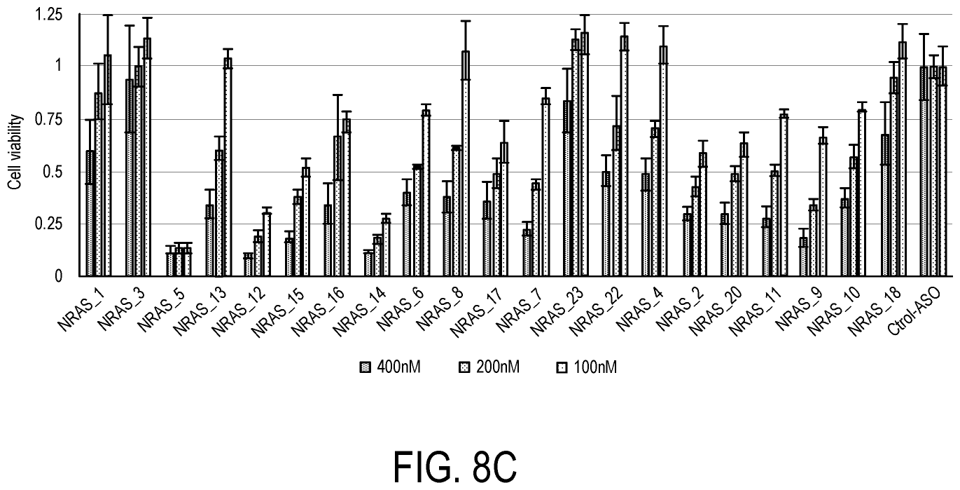

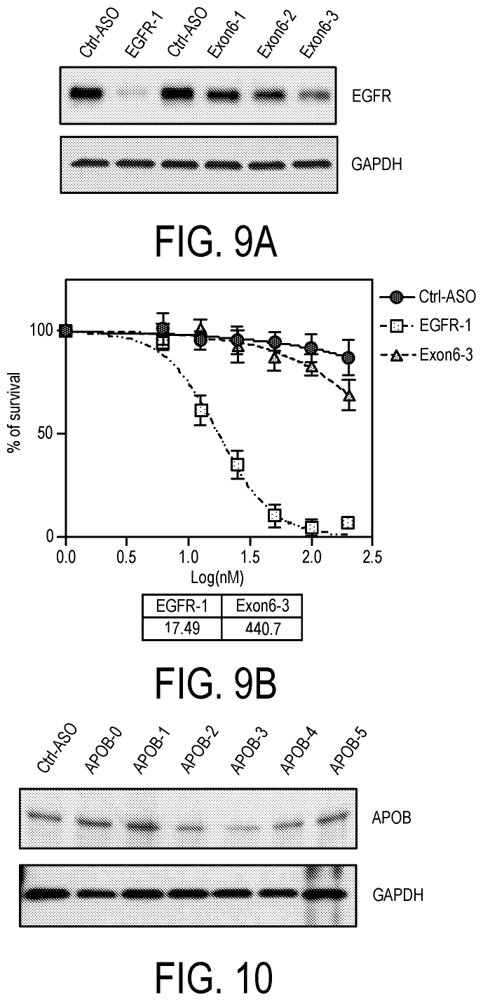

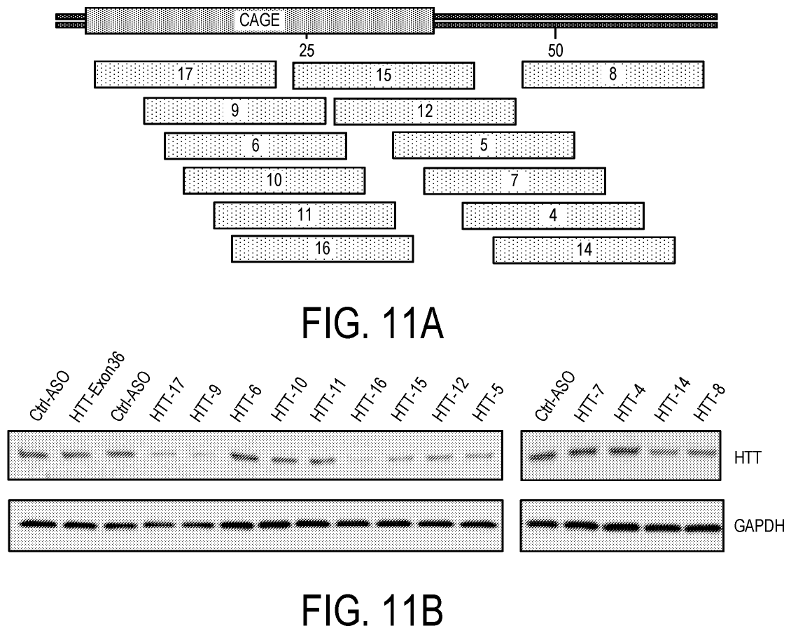

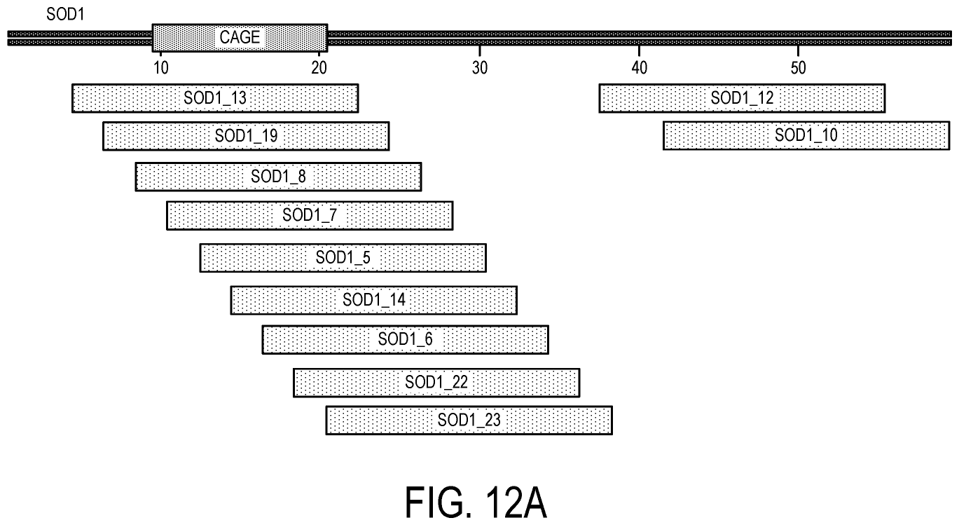

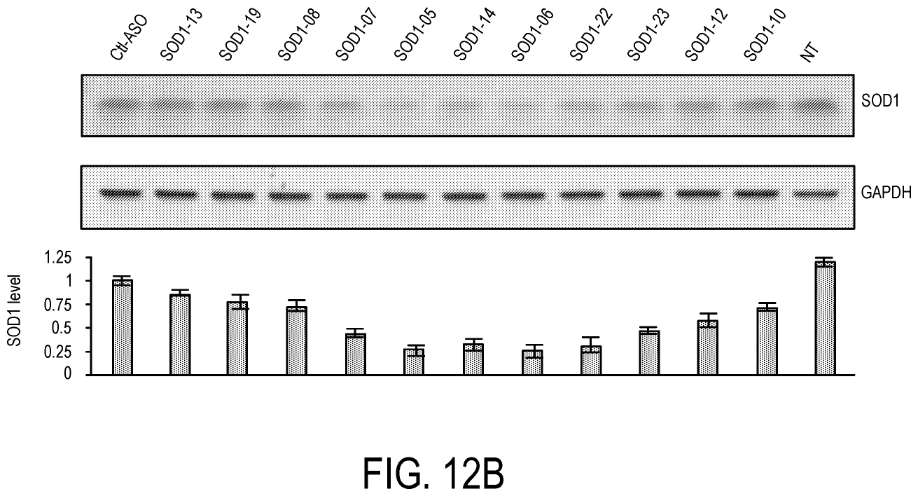

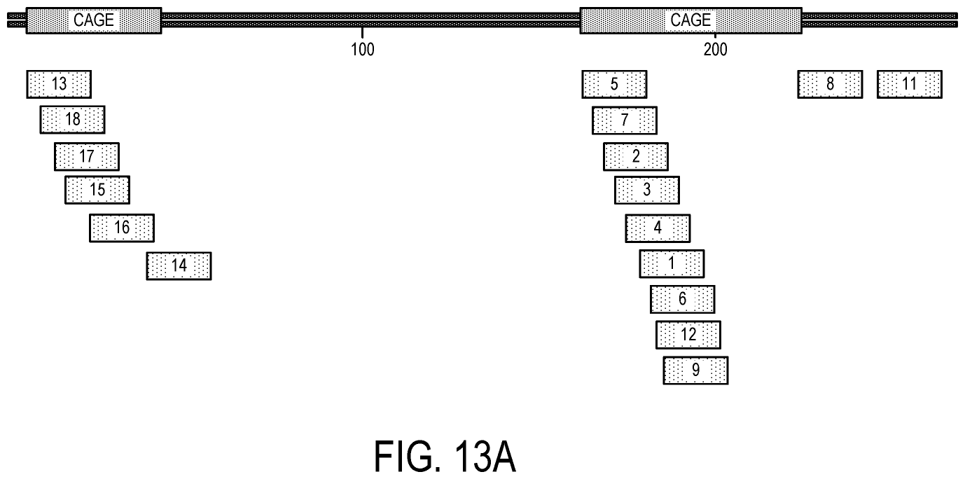

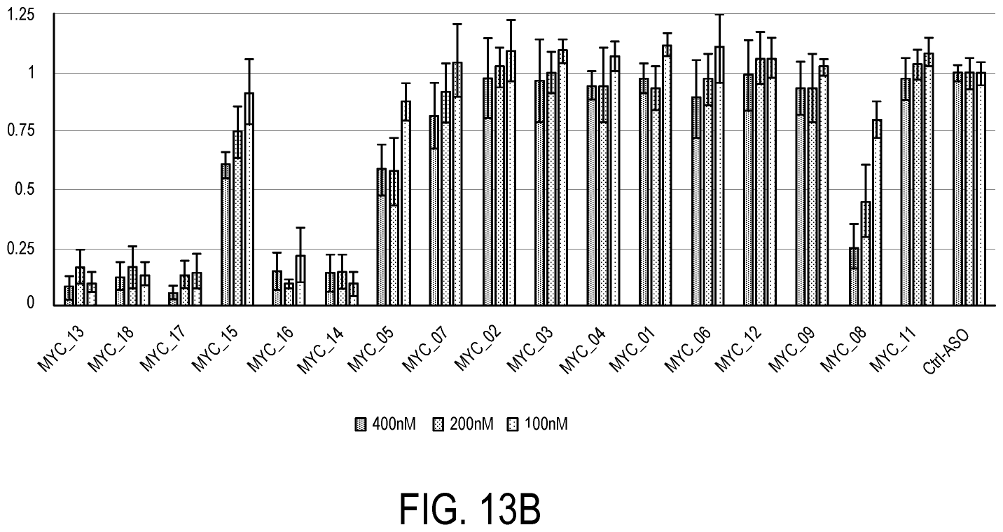

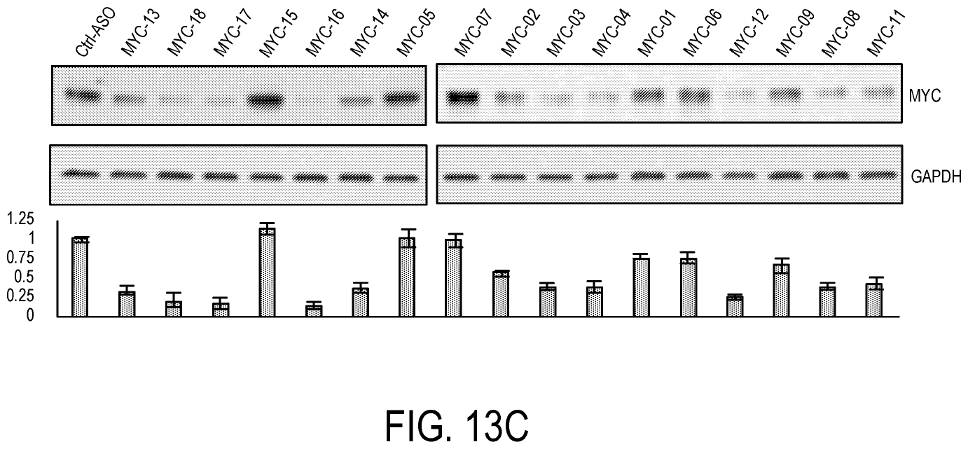

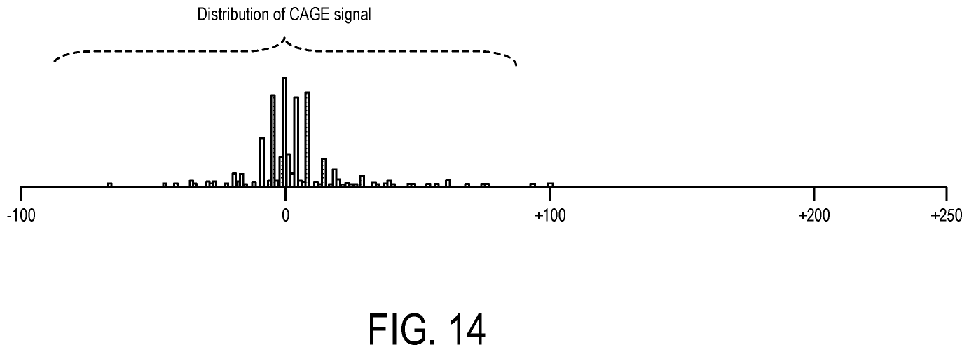

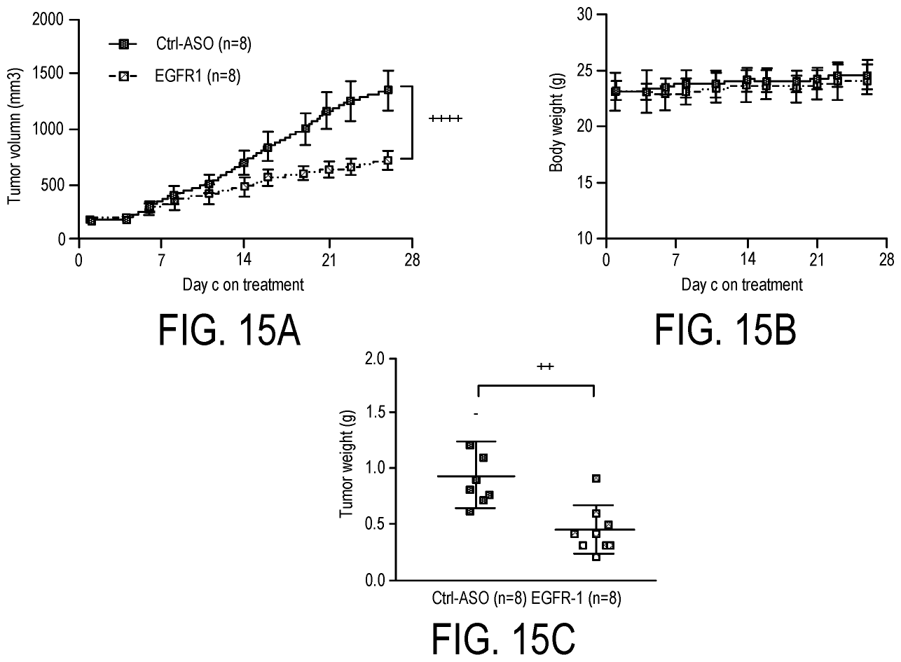

Elucidating the differences between INTi-mediated silencing of an oncogene and inhibition of its enzymatic activity? To further assess the degree of specificity in silencing of the key MAPK component in cancer cells, the transcript landscape is compared using RNA-seq following INTmer treatment and before/after exposure to small molecule inhibitors to different components of MAPK signaling, including MEK, ERK1/2, BRAF. As shown in FIG. 6 for EGFR protein, there are profound differences in silencing the expression of an oncogene verses its enzymatic inhibition (third generation inhibitor, WZ4002; similar to Osimertinib). Unlike treatment of EGFR mutant lung cancer cells treated with an EGFR inhibitor, silencing of EGFR leads to activation of DNA damage as shown by increased gama-H2AX phosphorylation and induction of apoptosis (increased PARP cleavage and caspase 3 activation) (FIGS. 6A and 6B). Importantly, these effects are specific to an INTmer against EGFR against start site sequences as a mutant INTmer decreasing the complementarity of the INTmer substantially decreased its silencing activity as well as induction of DNA damage and apoptosis (FIG. 6C). In case of BRAF, specific inhibitors are utilized to target mutant BRAF (Debrafenib). In case of NRAS, the INTmer responses to the targeted inhibition of MEK (Trametinib) and ERK1/2 are compared using specific inhibitors.

As shown in FIG. 6A, treatment of lung cancer cells with INTmers result in induction of apoptosis within 24 hours of treatment. Cancer cells with activating mutations in EGFR, NRAS or BRAF are treated with INTmers against each oncogene respectively and cell viability is assessed using a time-course experiments. A similar time-course is used to determine whether abrogation of the oncogene induce apoptotic response by measuring PARP cleavage and activated Caspase 3. The experiments using INTmers are compared with similar experiments using inhibitors of EGFR, BRAF, MEK and ERK1/2 to assess the phenotypic consequences of enzymatic inhibition vs silencing of gene expression.

### Example 5. Optimizing INTmers for In Vivo Use Through Oligo Modifications and Encapsulation by Lipid Nanoparticles

While, there have been several successes in the development of systemically administered ASOs in non-oncological settings, for example, FDA approved mipomersen (Kynamro) as the first systemically administered ASO drug to treat homozygous familial hypercholesterolemia, there have been major barriers in ASO delivery in oncology. Additionally, despite their specificity and broadness of use, there are some practical challenges in antisense pharmacology, such as improving the stability against nucleases degradation and increasing cellular delivery. To overcome nucleases degradation activity, second and third-generation oligonucleotides have been developed presenting structural modifications in the phosphate backbone or in the nucleotide sugar. For example, a next-generation class of ASOs that use 2′-4′ constrained ethyl (cEt) residues exhibit enhanced in vitro and in vivo potency compared to earlier ASO molecules. TSS of EGFR and BRAF oncogenes in lung and skin cancer cell lines with activating mutations in these oncogenes was targeted using 2MOE-GAPmers which are considered the second generation ASOs. Experiments are performed to examine additional proprietary chemistry (cET) often called third generation derivatized antisense oligonucleotides that reduce expression of target mRNA in human cancers suitable for use in vivo.

The other obstacle for oligonucleotide therapy is the poor ability to cross biological membranes caused by the characteristics of ASOs, such as high molecular weight (approximately 6-8 kDa), their hydrophilic nature and multiple negative charges. Also, the sites of action for oligonucleotides lay within the intracellular space, therefore, they need to overcome several biological barriers to reach their pharmacological targets in vivo. A wide variety of delivery platforms has been established to improve the pharmacokinetic properties of ASOs, either by direct conjugation to carriers or incorporation into nanoparticulate carriers. Among nanoparticle delivery systems at various stages of development, formulation with lipids is one of the most common approaches. Mixing polyanionic nucleic acid drugs with lipids leads to the condensing of nucleic acids into nanoparticles that have a more favorable surface charge, and are sufficiently large (˜100 nm in diameter) to trigger uptake by endocytosis. An important component of this work is the successful use of lipid-based nanoparticles (LNP), which have recently been used for delivery of siRNAs and CRISPR technology in human patients. In this study, two types of ASO nanoparticles, lipoplexes and liposomes, are manufactured and the ability for ASO delivery is assessed.

Lipoplexes, one type of lipid-based nanocarriers, are the complexes of DNA condensed with cationic lipids and polymers. Lipoplex formulations are typically a heterogeneous population of relatively unstable complexes which need to be prepared shortly before use, and have been successfully used for local delivery applications. To improve the stability of lipoplex nanoparticles, PLGA (poly-D, L lactide-co glycolide) based double emulsion method is used. PLGA is one of the best candidates for drug delivery because of its unique properties including biocompatibility, bioavailability and variable degradation kinetics, high drug-loading capability, stability and extended drug release over other carriers such as liposomes. There are several FDA approved products using PLGA as carriers which include Nutropin Depot for growth deficiencies, Sandostatin LAR for acromegaly and Trelstar Depot for prostate cancer. The PLGA nanoparticles can be prepared by different techniques. The most common technique is the emulsification solvent evaporation technique because of its simplicity, however, high encapsulation efficiency is hard to achieve in some cases. The double emulsion method is a good candidate to solve this problem, which may provide the simultaneous prolonged and high initial burst release of the drug. In brief, ASO mixes with poly-ethyleneimine (PEI) and then the mixture is emulsified with PLGA biodegradable polymers. The primary product is further emulsified with polyvinyl alcohol (PVA).

Liposome, the other type of lipid-based nanoparticle, is a spherical vesicle with at least one lipid bilayer, with the nucleic acid drug residing in the encapsulated aqueous space. Liposomes are more complex and exhibit more consistent physical properties with greater stability than lipoplexes. Some lipid nanoparticles (LNPs), also known as stable nucleic acid lipid particles, are liposomes that contain ionizable lipid, phosphatidylcholine, cholesterol and PEG-lipid conjugates in defined ratios and have been successfully utilized in multiple instances. In this study, a microfluidic mixing device Nanoassembler (Precision Nanosystems) is used, which adapted NxGen microfluidic technology and can provide easy, reproducible and controllable production of LNP. In brief, the lipid mixture (MC3, DSPC, Cholesterol, DMG-PEG, and DSPE-PEG) in ethanol and ASOs in an acetate buffer are injected in mixing device. The resultant mixture is diluted and dialyzed against phosphate buffered saline (PBS) (pH 7.4) for further usage. To produce LNPs that harbor a functional group for conjugation, DSPE-PEG carboxyl will be added to the lipid mixture. This LNP platform provide the flexibility to develop a cell-specific ASO delivery by simply conjugating the structural moiety that can be recognized by a specific cell type/tissue to DSPE-PEG through its carboxyl group.

### Example 6. Targeting Oncogenes In Vivo

Using the results of these in vitro studies, ASOs are tested for their ability to reduce cellular proliferation and expression of selected target genes in vivo using cell line-derived xenografts with activating mutation in BRAF, NRAS, HRAS and EGFR and their complementary patient-derived xenograft models (PDX) in immunocompromised mice. In these models, human tumor cells are transplanted, either subcutaneously or into the organ of origin. This study shows that transcriptional initiation by INTmers in vivo can result in a therapeutic outcome, thereby establishing a transformative approach to the treatment of cancer.

The in vitro findings are validated using two distinct preclinical PDX models for BRAF silencing in melanoma that are available in the laboratory. Similarly, EGFR mutant lung cancer models are used to assess the effectiveness of silencing EGFR in lung cancer. Cohorts of mice (n=8 mice per group) are exposed to Dabrafenib/Trametinib (DT) alone (this treatment is one standard of care for the treatment of BRAF-mutant melanoma patients) or the selected INTmers given either alone in phosphate-buffered saline (PBS) or as lipid nanoparticles encapsulation (a maximum of 3 concentrations will be tried). A sample is collected in each mouse using a small needle biopsy before the start of the treatment (TO samples). A minimum of three different samples is collected at minimal residual disease (MRD) (time point defined as no more decrease in tumor volume for 5 consecutive days) for every combination. Emergence of MRD is predominantly responsible for emergence of drug resistance to targeted therapy such as DT.

This study assesses the extent at which the tumor is diminished by the selected INTmers compared to the DT treatment alone. It has been shown that treatment of PDX models with DT result in emergence of four distinct cell population including a neural crest stem like (NCSC), staved-like, an invasive mesenchymal melanoma and a pigmented melanoma. The cellular composition of MRD is monitored by measuring the expression of various drug-tolerant gene expression mini-signatures by RT-qPCR and IHC. For the latter experiments, a 7-plex OPAL-based method is developed allowing the co-detection of MITF, CD36, AXL, NGFR, SOX10 and AQP1 on a single MRD slide. Quantification of the various drug-tolerant cell populations is performed using an AI-based methodology developed in-house. Signal quantification is performed on >500 cells in 10 different fields from at least 3 different biological replicates. These experiments are complemented by single-cell RNA-seq experiment on samples collected from the 2 most promising INTmers. These experiments identify the INTmers that are most efficient in eradicating the tumor as well as the emergence of MRD.

### Example 7. Innovation

Initiation of transcription interference (INTi) is a newly developed gene silencing platform that targets mRNA sequences that correspond to early phases of transcription initiation (nucleotides 20 to approximately 100 from transcriptional start site). These include RNA sequences corresponding to transcriptional initiation mechanisms consisting of promoter-escape and RNAPII pause-release. Importantly, INTmers are designed for use in vivo and therefore a potent INTmer against an oncogene such as NRAS constitute a potent drug against cancers with activated oncogene. INTi provides numerous technical advantages in gene silencing that can be explored for inhibiting disease-causing transcripts. The advantages are shown below:
1—As mentioned in the above paragraph, since the targeted area is around a 100 nucleotides, INTmers are easier to design and an initial screening of about 20 INTmers is sufficient to arrive at an affective INTmer. This is in contrast to current methods of screening thousands of ASOs for clinical use.
2—INTmers display high specific activity. Since the RNase H1-mediated cleavage occurs close to 5′-CAP structure, upon removal of the CAP structure the mRNA is targeted for nuclease degradation and decreased stability.
3—The targeting of transcriptional start sites allows for isoform-specific silencing of gene expression for transcripts with alternative upstream start sites. This is particularly important in cases where disease-specific or tissue-specific isoforms are targeted allowing for silencing of disease-isoform leaving the normal transcript intact such as shown in the principle example in silencing BRAF in melanoma (FIG. 5).
4—Finally, INTmer-mediated silencing of gene expression occurs within 8 to 10 hours following treatment (as compared to RNA interference which is typically takes 48 to 72 hours to function), reflective of a mechanism of transcriptional silencing consistent with premature termination of nascent transcript and de-capping.

Transcription Checkpoint Therapy is the future of RNA therapeutics: The findings lead to a transformative change in application of RNA therapeutics in human disease. While therapeutic oligonucleotides have garnered increased attention in the last few years, the advent of INTi allows access to potent disease-specific gene silencing technology that can be applied for drug development. The critical importance of this technology entails its wide application to diverse human conditions caused by pathogenic gene expression. These includes diseases such as Alzheimer, Parkinson or ALS for which there are no current therapeutic treatments.

TABLE 1
CAGE regions for INTmer design.

| CAGE | | Chromo- | | | | |
|---|---|---|---|---|---|---|
| region | Genomic sequence | some | Strand | Start | End* | Span |
| EGFR | CCTCGCATTCTCCTCCTCCTCTGCTCCT | chr7 | + | 55086610 | 55086959 | 350 |
| | CCCGATCCCTCCTCCGCCGCCTGGTCC | | | | | |
| | CTCCTCCTCCCGCCCTGCCTCCCCGCGC | | | | | |
| | CTCGGCCCGCGCGAGCTAGACGTCCGG | | | | | |
| | GCAGCCCCCGGCGCAGCGCGGCCGCA | | | | | |
| | GCAGCCTCCGCCCCCCGCACGGTGTGA | | | | | |
| | GCGCCCGACGCGGCCGAGGCGGCCGG | | | | | |
| | AGTCCCGAGCTAGCCCCGGCGGCCGCC | | | | | |
| | GCCGCCCAGACCGGACGACAGGCCAC | | | | | |
| | CTCGTCGGCGTCCGCCCGAGTCCCCGC | | | | | |
| | CTCGCCGCCAACGCCACAACCACCGCG | | | | | |
| | CACGGCCCCCTGACTCCGTCCAGTATT | | | | | |
| | GATCGGGAGAGCCGGAGCGAGCTCTTC | | | | | |
| | (SEQ ID NO: 1) | | | | | |
| BRAF | ACCGTGACACGCGGGTGACGCCGTTGC | chr7 | − | 140624471 | 140624820 | 350 |
| (Long | CGCGGCGACTTCTCGTCGTCTCCGCCC | | | | | |
| isoform) | CCTTCCCCCGCTCCCCCCCGCACCCCC | | | | | |
| | GCCTAGCGTCCTTCCCCCAATCCCCTC | | | | | |
| | AGGCTCGGCTGCGCCCGGGGCCGCGG | | | | | |
| | GCCGGTACCTGAGGTGGCCCAGGCGCC | | | | | |
| | CTCCGCCCGCGGCGCCGCCCGGGCCGC | | | | | |
| | TCCTCCCCGCGCCCCCCGCGCCCCCCG | | | | | |
| | CTCCTCCGCCTCCGCCTCCGCCTCCGCC | | | | | |
| | TCCCCCAGCTCTCCGCCTCCCTTCCCCC | | | | | |
| | TCCCCGCCCGACAGCGGCCGCTCGGGC | | | | | |
| | CCCGGCTCTCGGTTATAAGATGGCGGC | | | | | |
| | GCTGAGCGGTGGCGGTGGTGGCGGC | | | | | |
| | (SEQ ID NO: 2) | | | | | |
| BRAF | CCCCCCGCTCCTCCGCCTCCGCCTCCGC | chr7 | − | 140624263 | 140624612 | 350 |
| (Short | CTCCGCCTCCCCCAGCTCTCCGCCTCCC | | | | | |
| isoform) | TTCCCCCTCCCCGCCCGACAGCGGCCG | | | | | |
| | CTCGGGCCCCGGCTCTCGGTTATAAGA | | | | | |
| | TGGCGGCGCTGAGCGGTGGCGGTGGTG | | | | | |
| | GCGGCGCGGAGCCGGGCCAGGCTCTGT | | | | | |
| | TCAACGGGGACATGGAGCCCGAGGCC | | | | | |
| | GGCGCCGGCGCCGGCGCCGCGGCCTCT | | | | | |
| | TCGGCTGCGGACCCTGCCATTCCGGAG | | | | | |
| | GAGGTGAGTGCTGGCGCCACCCTGCCG | | | | | |
| | CCCTCCCGACTCCGGGCTCGGCGGCTG | | | | | |
| | GCTGGTGTTTATTTTGGAAAGAGGCGG | | | | | |
| | CGGTGGGGGCTTGATGCCCTCAGCC | | | | | |
| | (SEQ ID NO: 3) | | | | | |
| NRAS | GGTGCGGCCTGCCGCATGACTCGTGGT | chr1 | − | 115259136 | 115259485 | 350 |
| | TCGGAGGCCCACGTGGCCGGGGGGGG | | | | | |
| | GACTCAGGCGCCTGGGGCGCCGACTGA | | | | | |
| | TTACGTAGCGGGCGGGGCCGGAAGTG | | | | | |
| | CCGCTCCTTGGTGGGGGCTGTTCATGG | | | | | |
| | CGGTTCCGGGGTCTCCAACATTTTTCCC | | | | | |
| | GGCTGTGGTCCTAAATCTGTCCAAAGC | | | | | |
| | AGAGGCAGTGGAGCTTGAGGTAAGTTT | | | | | |
| | ATCTCATGCATAGTGTTCGGCTTTGGG | | | | | |
| | CTGTGGAATGTTCAGGCGTTTCACTGA | | | | | |
| | TGCCAGAAATGGAGCAGAATCTATCAG | | | | | |
| | CTGGAGACAAAGGCCTTGGGCGGGGG | | | | | |
| | TCCTTCCATTTGGTGCCTACGTGGGGA | | | | | |
| | G (SEQ ID NO: 4) | | | | | |
| Apoli- | AACAGGTCAGGCCCGGGAGGCGCCCTT | chr2 | − | 21266694 | 21267043 | 350 |
| poprotein | TGGACCTTTTGCAATCCTGGCGCTCTTG | | | | | |
| B-100 | CAGCCTGGGCTTCCTATAAATGGGGTG | | | | | |
| (APOB-100) | CGGGCGCCGGCCGCGCATTCCCACCGG | | | | | |
| | GACCTGCGGGGCTGAGTGCCCTTCTCG | | | | | |
| | GTTGCTGCCGCTGAGGAGCCCGCCCAG | | | | | |
| | CCAGCCAGGGCCGCGAGGCCGAGGCC | | | | | |
| | AGGCCGCAGCCCAGGAGCCGCCCCAC | | | | | |
| | CGCAGCTGGCGATGGACCCGCCGAGG | | | | | |
| | CCCGCGCTGCTGGCGCTGCTGGCGCTG | | | | | |
| | CCTGCGCTGCTGCTGCTGCTGCTGGCG | | | | | |
| | GGCGCCAGGGCCGGTGAGTGCGCGGC | | | | | |
| | CGCTCTGCGGGCGCAGAGGGAGCGGG | | | | | |
| | AGG (SEQ ID NO: 5) | | | | | |
| Huntingtin | TAGGGCTGTCAATCATGCTGGCCGGCG | chr4 | + | 3076312 | 3076661 | 350 |
| (HTT) | TGGCCCCGCCTCCGCCGGCGCGGCCCC | | | | | |
| | GCCTCCGCCGGCGCAGCGTCTGGGACG | | | | | |
| | CAAGGCGCCGTGGGGGCTGCCGGGAC | | | | | |
| | GGGTCCAAGATGGACGGCCGCTCAGGT | | | | | |
| | TCTGCTTTTACCTGCGGCCCAGAGCCC | | | | | |
| | CATTCATTGCCCCGGTGCTGAGCGGCG | | | | | |
| | CCGCGAGTCGGCCCGAGGCCTCCGGGG | | | | | |
| | ACTGCCGTGCCGGGCGGGAGACCGCC | | | | | |
| | ATGGCGACCCTGGAAAAGCTGATGAA | | | | | |
| | GGCCTTCGAGTCCCTCAAGTCCTTCCA | | | | | |
| | GCAGCAGCAGCAGCAGCAGCAGCAGC | | | | | |
| | AGCAGCAGCAGCAGCAGCAGCAGCAG | | | | | |
| | CAGC (SEQ ID NO: 6) | | | | | |
| Superoxide | GGGCGGGCTGAGCGCGTGCGAGGCGA | chr21 | + | 33031906 | 33032255 | 350 |
| Dismutase 1 | TTGGTTTGGGGCCAGAGTGGGCGAGGC | | | | | |
| (SOD1) | GCGGAGGTCTGGCCTATAAAGTAGTCG | | | | | |
| | CGGAGACGGGGTGCTGGTTTGCGTCGT | | | | | |
| | AGTCTCCTGCAGCGTCTGGGGTTTCCG | | | | | |
| | TTGCAGTCCTCGGAACCAGGACCTCGG | | | | | |
| | CGTGGCCTAGCGAGTTATGGCGACGAA | | | | | |
| | GGCCGTGTGCGTGCTGAAGGGCGACG | | | | | |
| | GCCCAGTGCAGGGCATCATCAATTTCG | | | | | |
| | AGCAGAAGGCAAGGGCTGGGACGGAG | | | | | |
| | GCTTGTTTGCGAGGCCGCTCCCACCCG | | | | | |
| | CTCGTCCCCCCGCGCACCTTTGCTAGG | | | | | |
| | AGCGGGTCGCCCGCCAGGCCTCGGGGC | | | | | |
| | CG (SEQ ID NO: 7) | | | | | |
| MYC | GGTTCCCAAAGCAGAGGGCGTGGGGG | chr8 | + | 128748216 | 128748565 | 350 |
| (Longer | AAAAGAAAAAAGATCCTCTCTCGCTAA | | | | | |
| isoform) | TCTCCGCCCACCGGCCCTTTATAATGC | | | | | |
| | GAGGGTCTGGACGGCTGAGGACCCCC | | | | | |
| | GAGCTGTGCTGCTCGCGGCCGCCACCG | | | | | |
| | CCGGGCCCCGGCCGTCCCTGGCTCCCC | | | | | |
| | TCCTGCCTCGAGAAGGGCAGGGCTTCT | | | | | |
| | CAGAGGCTTGGCGGGAAAAAGAACGG | | | | | |
| | AGGGAGGGATCGCGCTGAGTATAAAA | | | | | |
| | GCCGGTTTTCGGGGCTTTATCTAACTC | | | | | |
| | GCTGTAGTAATTCCAGCGAGAGGCAGA | | | | | |
| | GGGAGCGAGCGGGCGGCCGGCTAGGG | | | | | |
| | TGGAAGAGCCGGGCGAGCAGAGCTGC | | | | | |
| | GCTGC (SEQ ID NO: 8) | | | | | |
| MYC | TCCTGCCTCGAGAAGGGCAGGGCTTCT | chr8 | + | 128748376 | 128748725 | 350 |
| (Shorter | CAGAGGCTTGGCGGGAAAAAGAACGG | | | | | |
| isoform) | AGGGAGGGATCGCGCTGAGTATAAAA | | | | | |
| | GCCGGTTTTCGGGGCTTTATCTAACTC | | | | | |
| | GCTGTAGTAATTCCAGCGAGAGGCAGA | | | | | |
| | GGGAGCGAGCGGGCGGCCGGCTAGGG | | | | | |
| | TGGAAGAGCCGGGCGAGCAGAGCTGC | | | | | |
| | GCTGCGGGCGTCCTGGGAAGGGAGAT | | | | | |
| | CCGGAGCGAATAGGGGGCTTCGCCTCT | | | | | |
| | GGCCCAGCCCTCCCGCTGATCCCCCAG | | | | | |
| | CCAGCGGTCCGCAACCCTTGCCGCATC | | | | | |
| | CACGAAACTTTGCCCATAGCAGCGGGC | | | | | |
| | GGGCACTTTGCACTGGAACTTACAACA | | | | | |
| | CCCG (SEQ ID NO: 9) | | | | | |

Genomic location is based on Human Genome version 19 (hg19)

TABLE 2
Detailed information for screened INTmers.

| | | | Chromo- | | | | |
|---|---|---|---|---|---|---|---|
| ID | Name | Genomic sequence | some | Strand | Start* | End* | Span |
| TCS- | EGFR-1 | GCGGAGGCTGCTGCGGCC | chr7 | − | 55086739 | 55086756 | 18 |
| 0001 | | (SEQ ID NO: 10) | | | | | |
| TCS- | EGFR-2 | GGAGGCTGCTGCGGCCGC | chr7 | − | 55086737 | 55086754 | 18 |
| 0002 | | (SEQ ID NO: 11) | | | | | |
| TCS- | EGFR-3 | CGTCTAGCTCGCGCGGGC | chr7 | − | 55086697 | 55086714 | 18 |
| 0003 | | (SEQ ID NO: 12) | | | | | |
| TCS- | EGFR-4 | CTCACACCGTGCGGGGGG | chr7 | − | 55086756 | 55086773 | 18 |
| 0004 | | (SEQ ID NO: 13) | | | | | |
| TCS- | EGFR-5 | ACCGTGCGGGGGGCGGAG | chr7 | − | 55086751 | 55086768 | 18 |
| 0005 | | (SEQ ID NO: 14) | | | | | |
| TCS- | EGFR-6 | GACGTCTAGCTCGCGCGG | chr7 | − | 55086699 | 55086716 | 18 |
| 0006 | | (SEQ ID NO: 15) | | | | | |
| TCS- | EGFR-7 | GCTGCCCGGACGTCTAGC | chr7 | − | 55086707 | 55086724 | 18 |
| 0007 | | (SEQ ID NO: 16) | | | | | |
| TCS- | EGFR-8 | CACACCGTGCGGGGGGCG | chr7 | − | 55086754 | 55086771 | 18 |
| 0008 | | (SEQ ID NO: 17) | | | | | |
| TCS- | EGFR-9 | AGGCTGCTGCGGCCGCGC | chr7 | − | 55086735 | 55086752 | 18 |
| 0009 | | (SEQ ID NO: 18) | | | | | |
| TCS- | EGFR- | TGCTGCGGCCGCGCTGCG | chr7 | − | 55086731 | 55086748 | 18 |
| 0010 | 10 | (SEQ ID NO: 19) | | | | | |
| TCS- | EGFR- | GGGCTGCCCGGACGTCTA | chr7 | − | 55086709 | 55086726 | 18 |
| 0011 | 11 | (SEQ ID NO: 20) | | | | | |
| TCS- | EGFR- | GGGGGCTGCCCGGACGTC | chr7 | − | 55086711 | 55086728 | 18 |
| 0012 | 12 | (SEQ ID NO: 21) | | | | | |
| TCS- | EGFR- | GGCGGGAGTGGGGCGGCG | chr7 | − | 55086504 | 55086521 | 18 |
| 0013 | 13 | (SEQ ID NO: 22) | | | | | |
| TCS- | EGFR- | GCTGCGCCGGGGGCTGCC | chr7 | − | 55086719 | 55086736 | 18 |
| 0014 | 14 | (SEQ ID NO: 23) | | | | | |
| TCS- | EGFR- | TGCCCGGACGTCTAGCTC | chr7 | − | 55086705 | 55086722 | 18 |
| 0015 | 15 | (SEQ ID NO: 24) | | | | | |
| TCS- | EGFR- | CGGGAGGAGGAGGGACCA | chr7 | − | 55086659 | 55086676 | 18 |
| 0016 | 16 | (SEQ ID NO: 25) | | | | | |
| TCS- | EGFR- | CGGACGTCTAGCTCGCGC | chr7 | − | 55086701 | 55086718 | 18 |
| 0017 | 17 | (SEQ ID NO: 26) | | | | | |
| TCS- | EGFR- | GCCGGGGGCTGCCCGGAC | chr7 | − | 55086714 | 55086731 | 18 |
| 0018 | 18 | (SEQ ID NO: 27) | | | | | |
| TCS- | EGFR- | CCCGGACGTCTAGCTCGC | chr7 | − | 55086703 | 55086720 | 18 |
| 0019 | 19 | (SEQ ID NO: 28) | | | | | |
| TCS- | EGFR- | GGCAGGGCGGGAGGAGGA | chr7 | − | 55086666 | 55086683 | 18 |
| 0020 | 20 | (SEQ ID NO: 29) | | | | | |
| TCS- | EGFR- | CGCGGGGAGGCAGGGCGG | chr7 | − | 55086674 | 55086691 | 18 |
| 0021 | 21 | (SEQ ID NO: 30) | | | | | |
| TCS- | EGFR- | CGGGCCGAGGCGCGGGGA | chr7 | − | 55086684 | 55086701 | 18 |
| 0022 | 22 | (SEQ ID NO: 31) | | | | | |
| TCS- | EGFR- | GGAGGCAGGGCGGGAGGA | chr7 | − | 55086669 | 55086686 | 18 |
| 0023 | 23 | (SEQ ID NO: 32) | | | | | |
| TCS- | BRAF-1 | ATTGGGGGAAGGACGCTA | chr7 | + | 140624719 | 140624736 | 18 |
| 0024 | | (SEQ ID NO: 33) | | | | | |
| TCS- | BRAF-2 | AGGGGATTGGGGGAAGGA | chr7 | + | 140624714 | 140624731 | 18 |
| 0025 | | (SEQ ID NO: 34) | | | | | |
| TCS- | BRAF-3 | GGGATTGGGGGAAGGACG | chr7 | + | 140624716 | 140624733 | 18 |
| 0026 | | (SEQ ID NO: 35) | | | | | |
| TCS- | BRAF-4 | CTGAGGGGATTGGGGGAA | chr7 | 1 | 140624711 | 140624728 | 18 |
| 0027 | | (SEQ ID NO: 36) | | | | | |
| TCS- | BRAF-5 | TGGGGGAAGGACGCTAGG | chr7 | + | 140624721 | 140624738 | 18 |
| 0028 | | (SEQ ID NO: 37) | | | | | |
| TCS- | BRAF-6 | GAGCCTGAGGGGATTGGG | chr7 | + | 140624707 | 140624724 | 18 |
| 0029 | | (SEQ ID NO: 38) | | | | | |
| TCS- | BRAF-7 | CCGAGCCTGAGGGGATTG | chr7 | + | 140624705 | 140624722 | 18 |
| 0030 | | (SEQ ID NO: 39) | | | | | |
| TCS- | BRAF-8 | GGGAAGGACGCTAGGCGG | chr7 | + | 140624724 | 140624741 | 18 |
| 0031 | | (SEQ ID NO: 40) | | | | | |
| TCS- | BRAF-9 | AGCCGAGCCTGAGGGGAT | chr7 | + | 140624703 | 140624720 | 18 |
| 0032 | | (SEQ ID NO: 41) | | | | | |
| TCS- | BRAF- | GCCTGAGGGGATTGGGGG | chr7 | + | 140624709 | 140624726 | 18 |
| 0033 | 10 | (SEQ ID NO: 42) | | | | | |
| TCS- | BRAF- | TCAGGTACCGGCCCGCGG | chr7 | + | 140624675 | 140624692 | 18 |
| 0034 | 11 | (SEQ ID NO: 43) | | | | | |
| TCS- | BRAF- | CCGGGCGCAGCCGAGCCT | chr7 | + | 140624695 | 140624712 | 18 |
| 0035 | 12 | (SEQ ID NO: 44) | | | | | |
| TCS- | BRAF- | GCAGCCGAGCCTGAGGGG | chr7 | + | 140624701 | 140624718 | 18 |
| 0036 | 13 | (SEQ ID NO: 45) | | | | | |
| TCS- | BRAF- | GCGCAGCCGAGCCTGAGG | chr7 | + | 140624699 | 140624716 | 18 |
| 0037 | 14 | (SEQ ID NO: 46) | | | | | |
| TCS- | BRAF- | GGGCGCAGCCGAGCCTGA | chr7 | + | 140624697 | 140624714 | 18 |
| 0038 | 15 | (SEQ ID NO: 47) | | | | | |
| TCS- | BRAF- | GGCGGAGGGCGCCTGGGC | chr7 | + | 140624653 | 140624670 | 18 |
| 0039 | 16 | (SEQ ID NO: 48) | | | | | |
| TCS- | BRAF- | CGGAGGGCGCCTGGGCCA | chr7 | + | 140624655 | 140624672 | 18 |
| 0040 | 17 | (SEQ ID NO: 49) | | | | | |
| TCS- | BRAF- | CCCCGGGCGCAGCCGAGC | chr7 | + | 140624693 | 140624710 | 18 |
| 0041 | 18 | (SEQ ID NO: 50) | | | | | |
| TCS- | BRAF- | CTTATAACCGAGAGCCGG | chr7 | + | 140624504 | 140624521 | 18 |
| 0042 | 19 | (SEQ ID NO: 51) | | | | | |
| TCS- | BRAF- | GCGCCGCCATCTTATAAC | chr7 | + | 140624494 | 140624511 | 18 |
| 0043 | 20 | (SEQ ID NO: 52) | | | | | |
| TCS- | BRAF- | CATGTCCCCGTTGAACAG | chr7 | + | 140624435 | 140624452 | 18 |
| 0044 | 21 | (SEQ ID NO: 53) | | | | | |
| TCS- | BRAF- | ATCTTATAACCGAGAGCC | chr7 | + | 140624502 | 140624519 | 18 |
| 0045 | 22 | (SEQ ID NO: 54) | | | | | |
| TCS | BRAF- | CCCCGTTGAACAGAGCCT | chr7 | + | 140624440 | 140624457 | 18 |
| 0046 | 23 | (SEQ ID NO: 55) | | | | | |
| TCS- | NRAS-1 | CCGCCCGCTACGTAATCA | chr1 | + | 115259391 | 115259408 | 18 |
| 0047 | | (SEQ ID NO: 56) | | | | | |
| TCS | NRAS-2 | GCCATGAACAGCCCCCAC | chr1 | + | 115259352 | 115259369 | 18 |
| 0048 | | (SEQ ID NO: 57) | | | | | |
| TCS- | NRAS-3 | CCCCGCCCGCTACGTAAT | chr1 | + | 115259389 | 115259406 | 18 |
| 0049 | | (SEQ ID NO: 58) | | | | | |
| TCS- | NRAS-4 | CATGAACAGCCCCCACCA | chr1 | + | 115259354 | 115259371 | 18 |
| 0050 | | (SEQ ID NO: 59) | | | | | |
| TCS- | NRAS-5 | GGCCCCGCCCGCTACGTA | chr1 | + | 115259387 | 115259404 | 18 |
| 0051 | | (SEQ ID NO: 60) | | | | | |
| TCS- | NRAS-6 | CAAGGAGCGGCACTTCCG | chrl | + | 115259370 | 115259387 | 18 |
| 0052 | | (SEQ ID NO: 61) | | | | | |
| TCS- | NRAS-7 | CCCCCACCAAGGAGCGGC | chr1 | + | 115259363 | 115259380 | 18 |
| 0053 | | (SEQ ID NO: 62) | | | | | |
| TCS- | NRAS-8 | ACCAAGGAGCGGCACTTC | chr] | + | 115259368 | 115259385 | 18 |
| 0054 | | (SEQ ID NO: 63) | | | | | |
| TCS- | NRAS-9 | GCCGGGAAAAATGTTGGA | chr1 | + | 115259322 | 115259339 | 18 |
| 0055 | | (SEQ ID NO: 64) | | | | | |
| TCS- | NRAS- | GATTTAGGACCACAGCCG | chr1 | + | 115259308 | 115259325 | 18 |
| 0056 | 10 | (SEQ ID NO: 65) | | | | | |
| TCS- | NRAS- | AAAATGTTGGAGACCCCG | chr1 | + | 115259329 | 115259346 | 18 |
| 0057 | 11 | (SEQ ID NO: 66) | | | | | |
| TCS- | NRAS- | GGCACTTCCGGCCCCGCC | chrl | + | 115259378 | 115259395 | 18 |
| 0058 | 12 | (SEQ ID NO: 67) | | | | | |
| TCS- | NRAS- | TTCCGGCCCCGCCCGCTA | chr1 | + | 115259383 | 115259400 | 18 |
| 0059 | 13 | (SEQ ID NO: 68) | | | | | |
| TCS- | NRAS- | AGGAGCGGCACTTCCGGC | chr1 | + | 115259372 | 115259389 | 18 |
| 0060 | 14 | (SEQ ID NO: 69) | | | | | |
| TCS- | NRAS- | GCGGCACTTCCGGCCCCG | chr1 | + | 115259376 | 115259393 | 18 |
| 0061 | 15 | (SEQ ID NO: 70) | | | | | |
| TCS- | NRAS- | GAGCGGCACTTCCGGCCC | chr1 | + | 115259374 | 115259391 | 18 |
| 0062 | 16 | (SEQ ID NO: 71) | | | | | |
| TCS- | NRAS- | CCACCAAGGAGCGGCACT | chr1 | + | 115259366 | 115259383 | 18 |
| 0063 | 17 | (SEQ ID NO: 72) | | | | | |
| TCS- | NRAS- | GACAGATTTAGGACCACA | chr1 | + | 115259304 | 115259321 | 18 |
| 0064 | 18 | (SEQ ID NO: 73) | | | | | |
| TCS- | NRAS- | CCTCTGCTTTGGACAGAT | chr1 | + | 115259293 | 115259310 | 18 |
| 0065 | 19 | (SEQ ID NO: 74) | | | | | |
| TCS- | NRAS- | CGGAACCGCCATGAACAG | chr1 | + | 115259345 | 115259362 | 18 |
| 0066 | 20 | (SEQ ID NO: 75) | | | | | |
| TCS- | NRAS- | GCTTTGGACAGATTTAGG | chr1 | + | 115259298 | 115259315 | 18 |
| 0067 | 21 | (SEQ ID NO: 76) | | | | | |
| TCS- | NRAS- | GAACAGCCCCCACCAAGG | chr1 | + | 115259357 | 115259374 | 18 |
| 0068 | 22 | (SEQ ID NO: 77) | | | | | |
| TCS- | NRAS- | CAGCCCCCACCAAGGAGC | chr1 | + | 115259360 | 115259377 | 18 |
| 0069 | 23 | (SEQ ID NO: 78) | | | | | |
| TCS- | APOB-1 | GCCCCGCAGGTCCCGGTG | chr2 | + | 21266923 | 21266940 | 18 |
| 0070 | | (SEQ ID NO: 79) | | | | | |
| TCS- | APOB-2 | CTCAGCCCCGCAGGTCCC | chr2 | + | 21266919 | 21266936 | 18 |
| 0071 | | (SEQ ID NO: 80) | | | | | |
| TCS- | APOB-3 | ACCGAGAAGGGCACTCAG | chr2 | + | 21266906 | 21266923 | 18 |
| 0072 | | (SEQ ID NO: 81) | | | | | |
| TCS- | APOB-4 | CGGCAGCAACCGAGAAGG | chr2 | + | 21266898 | 21266915 | 18 |
| 0073 | | (SEQ ID NO: 82) | | | | | |
| TCS- | APOB-5 | TCAGCGGCAGCAACCGAG | chr2 | + | 21266894 | 21266911 | 18 |
| 0074 | | (SEQ ID NO: 83) | | | | | |
| TCS- | HTT-1 | CAATGAATGGGGCTCTGG | chr4 | − | 3076464 | 3076481 | 18 |
| 0075 | | (SEQ ID NO: 84) | | | | | |
| TCS- | HTT-2 | GGCAATGAATGGGGCTCT | chr4 | − | 3076466 | 3076483 | 18 |
| 0076 | | (SEQ ID NO: 85) | | | | | |
| TCS- | HTT-3 | ATGAATGGGGCTCTGGGC | chr4 | − | 3076462 | 3076479 | 18 |
| 0077 | | (SEQ ID NO: 86) | | | | | |
| TCS- | HTT-4 | CGCAGGTAAAAGCAGAAC | chr4 | − | 3076444 | 3076461 | 18 |
| 0078 | | (SEQ ID NO: 87) | | | | | |
| TCS- | HTT-5 | AAAAGCAGAACCTGAGCG | chr4 | − | 3076437 | 3076454 | 18 |
| 0079 | | (SEQ ID NO: 88) | | | | | |
| TCS- | HTT-6 | CCATCTTGGACCCGTCCC | chr4 | − | 3076414 | 3076431 | 18 |
| 0080 | | (SEQ ID NO: 89) | | | | | |
| TCS- | HTT-7 | GGTAAAAGCAGAACCTGA | chr4 | − | 3076440 | 3076457 | 18 |
| 0081 | | (SEQ ID NO: 90) | | | | | |
| TCS- | HTT-8 | CTGGGCCGCAGGTAAAAG | chr4 | − | 3076450 | 3076467 | 18 |
| 0082 | | (SEQ ID NO: 91) | | | | | |
| TCS- | HTT-9 | ATCTTGGACCCGTCCCGG | chr4 | − | 3076412 | 3076429 | 18 |
| 0083 | | (SEQ ID NO: 92) | | | | | |
| TCS- | HTT-10 | GTCCATCTTGGACCCGTC | chr4 | − | 3076416 | 3076433 | 18 |
| 0084 | | (SEQ ID NO: 93) | | | | | |
| TCS- | HTT-11 | GCCGTCCATCTTGGACCC | chr4 | − | 3076419 | 3076436 | 18 |
| 0085 | | (SEQ ID NO: 94) | | | | | |
| TCS- | HTT-12 | AGAACCTGAGCGGCCGTC | chr4 | − | 3076431 | 3076448 | 18 |
| 0086 | | (SEQ ID NO: 95) | | | | | |
| TCS- | HTT-13 | GAATGGGGCTCTGGGCCG | chr4 | − | 3076460 | 3076477 | 18 |
| 0087 | | (SEQ ID NO: 96) | | | | | |
| TCS- | HTT-14 | GGCCGCAGGTAAAAGCAG | chr4 | − | 3076447 | 3076464 | 18 |
| 0088 | | (SEQ ID NO: 97) | | | | | |
| TCS- | HTT-15 | CCTGAGCGGCCGTCCATC | chr4 | − | 3076427 | 3076444 | 18 |
| 0089 | | (SEQ ID NO: 98) | | | | | |
| TCS- | HTT-16 | CGGCCGTCCATCTTGGAC | chr4 | − | 3076421 | 3076438 | 18 |
| 0090 | | (SEQ ID NO: 99) | | | | | |
| TCS- | HTT-17 | GGACCCGTCCCGGCAGCC | chr4 | − | 3076407 | 3076424 | 18 |
| 0091 | | (SEQ ID NO: 100) | | | | | |
| TCS- | SOD1-1 | CGCGACTACTTTATAGGC | chr21 | − | 33031970 | 33031987 | 18 |
| 0092 | | (SEQ ID NO: 101) | | | | | |
| TCS- | SOD1-2 | TCCGCGACTACTTTATAG | chr21 | − | 33031972 | 33031989 | 18 |
| 0093 | | (SEQ ID NO: 102) | | | | | |
| TCS- | SOD1-3 | TTATAGGCCAGACCTCCG | chr21 | − | 33031960 | 33031977 | 18 |
| 0094 | | (SEQ ID NO: 103) | | | | | |
| TCS- | SOD1-4 | GTCTCCGCGACTACTTTA | chr21 | − | 33031975 | 33031992 | 18 |
| 0095 | | (SEQ ID NO: 104) | | | | | |
| TCS- | SOD1-5 | AGGAGACTACGACGCAAA | chr21 | − | 33032003 | 33032020 | 18 |
| 0096 | | (SEQ ID NO: 105) | | | | | |
| TCS- | SOD1-6 | CTGCAGGAGACTACGACG | chr21 | − | 33032007 | 33032024 | 18 |
| 0097 | | (SEQ ID NO: 106) | | | | | |
| TCS- | SOD1-7 | GAGACTACGACGCAAACC | chr21 | − | 33032001 | 33032018 | 18 |
| 0098 | | (SEQ ID NO: 107) | | | | | |
| TCS- | SOD1-8 | GACTACGACGCAAACCAG | chr21 | − | 33031999 | 33032016 | 18 |
| 0099 | | (SEQ ID NO: 108) | | | | | |
| TCS- | SOD1-10 | AGGACTGCAACGGAAACC | chr21 | − | 33032032 | 33032049 | 18 |
| 0100 | | (SEQ ID NO: 109) | | | | | |
| TCS- | SOD1-11 | CGACTACTTTATAGGCCA | chr21 | − | 33031968 | 33031985 | 18 |
| 0101 | | (SEQ ID NO: 110) | | | | | |
| TCS- | SOD1-12 | CTGCAACGGAAACCCCAG | chr21 | − | 33032028 | 33032045 | 18 |
| 0102 | | (SEQ ID NO: 111) | | | | | |
| TCS- | SOD1-13 | ACGACGCAAACCAGCACC | chr21 | − | 33031995 | 33032012 | 18 |
| 0103 | | (SEQ ID NO: 112) | | | | | |
| TCS- | SOD1-14 | GCAGGAGACTACGACGCA | chr21 | − | 33032005 | 33032022 | 18 |
| 0104 | | (SEQ ID NO: 113) | | | | | |
| TCS- | SOD1-15 | GACGCAAACCAGCACCCC | chr21 | − | 33031993 | 33032010 | 18 |
| 0105 | | (SEQ ID NO: 114) | | | | | |
| TCS- | SOD1-16 | ATAGGCCAGACCTCCGCG | chr21 | − | 33031958 | 33031975 | 18 |
| 0106 | | (SEQ ID NO: 115) | | | | | |
| TCS- | SOD1-17 | CGCAAACCAGCACCCCGT | chr21 | − | 33031991 | 33032008 | 18 |
| 0107 | | (SEQ ID NO: 116) | | | | | |
| TCS- | SOD1-19 | CTACGACGCAAACCAGCA | chr21 | − | 33031997 | 33032014 | 18 |
| 0108 | | (SEQ ID NO: 117) | | | | | |
| TCS- | SOD1-20 | CCGTCTCCGCGACTACTT | chr21 | − | 33031977 | 33031994 | 18 |
| 0109 | | (SEQ ID NO: 118) | | | | | |
| TCS- | SOD1-22 | CGCTGCAGGAGACTACGA | chr21 | − | 33032009 | 33032026 | 18 |
| 0110 | | (SEQ ID NO: 119) | | | | | |
| TCS- | SOD1-23 | GACGCTGCAGGAGACTAC | chr21 | − | 33032011 | 33032028 | 18 |
| 0111 | | (SEQ ID NO: 120) | | | | | |
| TCS- | MYC-1 | CGCTGGAATTACTACAGC | chr8 | − | 128748482 | 128748499 | 18 |
| 0112 | | GACGCTGCAGGAGACTAC | | | | | |
| | | (SEQ ID NO: 121) | | | | | |
| TCS- | MYC-2 | ACTACAGCGAGTTAGATA | chr8 | − | 128748472 | 128748489 | 18 |
| 0113 | | (SEQ ID NO: 122) | | | | | |
| TCS- | MYC-3 | ATTACTACAGCGAGTTAG | chr8 | − | 128748475 | 128748492 | 18 |
| 0114 | | (SEQ ID NO: 123) | | | | | |
| TCS- | MYC-4 | GGAATTACTACAGCGAGT | chr8 | − | 128748478 | 128748495 | 18 |
| 0115 | | (SEQ ID NO: 124) | | | | | |
| TCS- | MYC-5 | GCGAGTTAGATAAAGCCC | chr8 | − | 128748466 | 128748483 | 18 |
| 0116 | | (SEQ ID NO: 125) | | | | | |
| TCS- | MYC-6 | TCTCGCTGGAATTACTAC | chr8 | − | 128748485 | 128748502 | 18 |
| 0117 | | (SEQ ID NO: 126) | | | | | |
| TCS- | MYC-7 | ACAGCGAGTTAGATAAAG | chr8 | − | 128748469 | 128748486 | 18 |
| 0118 | | (SEQ ID NO: 127) | | | | | |
| TCS- | MYC-8 | GGCTCTTCCACCCTAGCC | chr8 | − | 128748527 | 128748544 | 18 |
| 0119 | | (SEQ ID NO: 128) | | | | | |
| TCS- | MYC-9 | TGCCTCTCGCTGGAATTA | chr8 | − | 128748489 | 128748506 | 18 |
| 0120 | | (SEQ ID NO: 129) | | | | | |
| TCS- | MYC-11 | CGCAGCGCAGCTCTGCTC | chr8 | − | 128748549 | 128748566 | 18 |
| 0121 | | (SEQ ID NO: 130) | | | | | |
| TCS- | MYC-12 | CCTCTCGCTGGAATTACT | chr8 | − | 128748487 | 128748504 | 18 |
| 0122 | | (SEQ ID NO: 131) | | | | | |
| TCS- | MYC-13 | AGCTCGGGGGTCCTCAGC | chr8 | − | 128748309 | 128748326 | 18 |
| 0123 | | (SEQ ID NO: 132) | | | | | |
| TCS- | MYC-14 | GCCGGGGCCCGGCGGTGG | chr8 | − | 128748343 | 128748360 | 18 |
| 0124 | | (SEQ ID NO: 133) | | | | | |
| TCS- | MYC-15 | GCGAGCAGCACAGCTCGG | chr8 | − | 128748320 | 128748337 | 18 |
| 0125 | | (SEQ ID NO: 134) | | | | | |
| TCS- | MYC-16 | GGCGGCCGCGAGCAGCAC | chr8 | − | 128748327 | 128748344 | 18 |
| 0126 | | (SEQ ID NO: 135) | | | | | |
| TCS- | MYC-17 | AGCAGCACAGCTCGGGGG | chr8 | − | 128748317 | 128748334 | 18 |
| 0127 | | (SEQ ID NO: 136) | | | | | |
| TCS- | MYC-18 | GCACAGCTCGGGGGTCCT | chr8 | − | 128748313 | 128748330 | 18 |
| 0128 | | (SEQ ID NO: 137) | | | | | |

*Genomic location is based on Human Genome version 19 (hg19)

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will appreciate that numerous changes and modifications can be made to the preferred embodiments of the invention and that such changes and modifications can be made without departing from the spirit of the invention. It is, therefore, intended that the appended claims cover all such equivalent variations as fall within the true spirit and scope of the invention.

What is claimed is:

1. A method of silencing expression of a gene of interest, the method comprising:
a. exposing a cell comprising the gene of interest to an Initiation of Transcription GAPmer (INTmer), wherein said INTmer comprises a nucleic acid molecule comprising 14-22 nucleotides, and further wherein said nucleic acid molecule comprises a central core of DNA flanked by RNA on each side, wherein the central core of DNA is complementary to at least a portion of an mRNA transcript of the gene of interest;
b. allowing the INTmer to hybridize to the nascent mRNA transcript of the gene of interest, wherein said INTmer hybridizes within the first 150 base pairs of a 5′ end of the nascent mRNA transcript; and
c. cleaving the nascent RNA:INTmer duplex, thereby silencing expression of the gene of interest.

2. The method of claim 1, wherein the gene of interest is a mutated gene.

3. The method of claim 1 or 2, wherein the gene of interest is an oncogene.

4. The method of any one of claims 1-3, wherein the gene of interest is EGFR, BRAF, NRAS, APOB-100, Huntingtin (HTT), SOD1, or MYC.

5. The method of any one of claims 1-4, wherein the cell is in vitro.

6. The method of any one of claims 1-5, wherein the cell is in vivo.

7. The method of any one of claims 1-6, wherein the INTmer is 14 nucleotides long.

8. The method of any one of claims 1-6, wherein the INTmer is 15 nucleotides long.

9. The method of any one of claims 1-6, wherein the INTmer is 16 nucleotides long.

10. The method of any one of claims 1-6, wherein the INTmer is 17 nucleotides long.

11. The method of any one of claims 1-6, wherein the INTmer is 18 nucleotides long.

12. The method of any one of claims 1-6, wherein the INTmer is 19 nucleotides long.

13. The method of any one of claims 1-6, wherein the INTmer is 20 nucleotides long.

14. The method of any one of claims 1-6, wherein the INTmer is 21 nucleotides long.

15. The method of any one of claims 1-6, wherein the INTmer is 22 nucleotides long.

16. The method of any one of claims 1-15, wherein the central core is 8, 9, 10, 11, or 12 nucleotides in length.

17. The method of any one of claims 1-16, wherein the central core comprises at least one modified nucleotide.

18. The method of claim 17, wherein said modified nucleotide comprises phosphorothioate and/or is 5-methylcytosine.

19. The method of any one of claims 1-18, wherein each flanking RNA is 2, 3, 4, or 5 nucleotides in length on either side of the central core.

20. The method of any one of claims 1-19, wherein the RNA of the INTmer is modified.

21. The method of claim 20, wherein the modified RNA comprises 2′-O-methoxy (MOE)-modified RNA.

22. The method of any one of claims 1-21, wherein the modified RNA comprises locked nucleic acid (LNA).

23. The method of any one of claims 1-22, wherein the nascent mRNA transcript is about 50, 100, 150, or 200 nucleotides in length when the INTmer hybridizes with it.

24. The method of any one of claims 1-23, wherein the INTmer hybridizes within 5, 10, 15, 20, 25, 30, 35, 40, 45, 50, 55, 60, 65, 70, 75, 80, 85, 90, 95, 100, or 150 nucleotides or less of the CAP structure of the nascent RNA.

25. The method of any one of claims 1-24, wherein the cleaved RNA transcript is de-CAPped.

26. The method of any one of claims 1-24, wherein the nascent RNA transcript is degraded by nuclease.

27. A method of treating a subject with aberrant gene expression, the method comprising silencing expression of the aberrantly transcribed gene by:
a. administering to the subject an Initiation of Transcription GAPmer (INTmer), wherein said INTmer comprises a nucleic acid molecule comprising 14-22 nucleotides, and further wherein said nucleic acid molecule comprises a central core of DNA flanked by RNA on each side, wherein the central core of DNA is complementary to at least a portion of an RNA transcript of the aberrantly transcribed gene;
b. allowing the INTmer to hybridize to the nascent RNA transcript of the aberrantly transcribed gene, wherein said INTmer within the first 150 base pairs of a 5′ end of the nascent mRNA transcript; and
c. allowing RNase H1 to cleave the nascent RNA:INTmer duplex, thereby silencing expression of the aberrantly transcribed gene.

28. The method of claim 27, wherein the aberrantly transcribed gene is a mutated gene or is aberrantly over-expressed.

29. The method of claim 27, wherein the aberrantly transcribed gene comprises an activating mutation.

30. The method of claim 28 wherein the mutated gene is an oncogene.

31. The method of any one of claims 28-30, wherein the mutated gene is EGFR, BRAF, NRAS, APOB-100, Huntingtin, SOD1, or MYC.

32. The method of any one of claims 27-31, wherein the INTmer is 14 nucleotides long.

33. The method of any one of claims 23-31, wherein the INTmer is 15 nucleotides long.

34. The method of any one of claims 23-31, wherein the INTmer is 16 nucleotides long.

35. The method of any one of claims 23-31, wherein the INTmer is 17 nucleotides long.

36. The method of any one of claims 23-31, wherein the INTmer is 18 nucleotides long.

37. The method of any one of claims 23-31, wherein the INTmer is 19 nucleotides long.

38. The method of any one of claims 23-31, wherein the INTmer is 20 nucleotides long.

39. The method of any one of claims 27-31, wherein the INTmer is 14 nucleotides long.

40. The method of any one of claims 27-31, wherein the INTmer is 14 nucleotides long.

41. The method of any one of claims 27-40, wherein the central core is 8, 9, 10, 11, or 12 nucleotides long.

42. The method of any one of claims 27-41, wherein the central core comprises at least one modified nucleotide.

43. The method of claim 42, wherein said modified nucleotide comprises phosphorothioate and/or is 5-methylcytosine.

44. The method of any one of claims 27-43, wherein each flanking nuclease-resistant RNA is 2, 3, 4, or 5 nucleotides in length on either side of the central core.

45. The method of any one of claims 27-44, wherein the RNA of the INTmer is modified.

46. The method of claim 45, wherein the RNA comprises 2′-O-methoxy (MOE)-modified RNA.

47. The method of claim 45, wherein the nuclease-resistant RNA comprises locked nucleic acid LNA.

48. The method of any one of claims 27-47, wherein the nascent RNA transcript is about 50, 100, 150, 200,250, 300, or 350 nucleotides in length.

49. The method of any one of claims 27-48, wherein the INTmer hybridizes within 5, 10, 15, 20, 25, 30, 35, 40, 45, 50, 55, 60, 65, 70, 75, 80, 85, 90, 95, 100, or 150 nucleotides or less of the CAP structure of the nascent RNA.

50. The method of any one of claims 27-49, wherein the cleaved RNA transcript is de-CAPped.

51. The method of any one of claims 27-50 wherein the nascent RNA transcript is degraded by nuclease.

52. The method of any one of claims 27-51, wherein the subject has cancer.

53. The method of any one of claims 27-51, wherein the subject has a chromosomal abnormality or the aberrant gene over-expressed.

54. The method of any one of claims 27-53, wherein the INTmer is administered via a carrier.

55. The method of claim 54, wherein the carrier is a nanoparticle, liposome, or exosome.

56. The method of any one of claims 27-55, wherein said INTmer is conjugated to another molecule to facilitate delivery.

57. The method of claim 56, wherein said molecule comprises LNPs, DPC™, TRiM™, or GalNAc.

58. A method for identifying INTmers which are capable of downregulating gene expression of a gene of interest, the method comprising:
a. sequencing at least the first 150 nucleotides of a region at a 5′ end of an RNA transcript of the gene of interest, or sequencing the gene of interest itself;
b. creating a library of INTmers which are at least 80% complementary to region within the first 150 nucleotides of the RNA transcript of the gene of interest sequenced in step a), wherein said INTmers comprises a nucleic acid molecule comprising 16-20 nucleotides, and further wherein said nucleic acid molecule comprises a central core of DNA flanked by RNA on each side;
c. exposing the INTmer library created in step b) to mammalian primary cells or cell lines expressing the nascent RNA transcript complementary to the INTmer sequencies; and
d. determining which INTmers are capable of downregulation of gene expression of the gene of interest.

59. The method of claim 58, wherein the INTmer is 90% or more complementary to the RNA transcript.

60. The method of claim 58 or 59, wherein the INTmer is 100% complementary to the RNA transcript.

61. The method of any one of claims 58-60, wherein the method is carried out in cells in vitro.

62. The method of claim 61, wherein the cells are cancer cells.

63. The method of any one of claims 58-62, wherein, in step d), determining which INTmers are capable of downregulation of gene expression is carried out by measuring RNA levels of transcripts of the gene of interest.

64. The method of claim 63, wherein RT-qPCR is used to measure RNA levels.

65. The method of any one of claims 58-64, wherein protein levels of a protein corresponding to a gene of interest are measured.

66. The method of claim 65, wherein protein levels are measured using Western Blotting.

67. The method of any one of claims 58-66, wherein cellular viability is measured.

68. The method of any one of claims 58-67, wherein the INTmer is designed against an RNA polymerase II pause-release domain.

69. The method of claim 68, wherein the pause-release domain corresponds to about nucleotides 40 to 150 of the nascent RNA transcript.

70. The method of any one of claims 58-67, wherein the INTmer is designed against an RNA polymerase promoter-escape region.

71. The method of claim 70, wherein the promoter-escape region corresponds to about nucleotides 20 to 80 of the nascent RNA transcript.

72. A composition comprising an INTmer, wherein the INTmer hybridizes to a CAGE region, wherein said CAGE region comprises any of SEQ ID NOS: 1-9.

73. A composition comprising an INTmer, wherein the INTmer comprises any of SEQ ID NOS: 10-137.

74. A composition comprising an INTmer, wherein the INTmer comprises at least 90% identity to any one SEQ ID NOS: 10-137.

75. The composition of any one of claims 72-74, wherein said INTmer comprises a modification.

76. The composition of claim 75, wherein said modification comprises a conjugated non-nucleic acid molecule.

77. The composition of claim 76, wherein said modification comprises α-tocopherol or cholesterol.
